(19) United States
(12) Patent Application Publication
(10) Pub. No.: US 2026/0242803 A1
(43) Pub. Date: Aug. 20, 2026

(54) RESPIRATORY VECTORS AND USES THEREOF

(71) Applicant: ALDEVRON, LLC, Fargo, ND (US)

(72) Inventors: Ricardo SALDANA-MEYER, Stoneham, MA (US); Jin HUH, Chapel Hill, NC (US); Jodi KENNEDY, Dedham, MA (US)

(21) Appl. No.: 18/993,971

(22) PCT Filed: Jul. 13, 2023
(86) PCT No.: PCT/IB2023/000544

§ 371 Date: Jan. 13, 2025

Related U.S. Application Data
(60) Provisional application No. 63/388,880, filed on Jul. 13, 2022.

(51) Int. Cl. C12N 15/64; A61K 9/51; A61K 31/711; A61K 31/713; C07K 14/705

(52) U.S. Cl. CPC C12N 15/64; A61K 9/5123; A61K 31/711; A61K 31/713; C07K 14/705; C12N 2310/532; C12N 2800/10; C12N 2830/34

(57) ABSTRACT

The application provides circular DNA vectors comprising promoters, scaffold matrix attachment region (S/MAR) sequences, and transgenes derived from proteins expressed in respiratory epithelium and which lack drug resistance genes. The application provides pharmaceutical compositions comprising therapeutic amounts of the DNA vectors.

### FIELD OF THE INVENTION

In general, the invention features nucleic acid vectors, e.g., respiratory nucleic acid vectors expressible in the airway.

### BACKGROUND

Gene therapy is emerging as a promising approach to treat a wide variety of diseases and disorders in human patients. There have been numerous attempts to apply gene therapy strategies to target respiratory tissues for treatment of disease, such as cystic fibrosis. However, these efforts have encountered significant setbacks due, in part, to inefficient (e.g., transient) gene expression and severe inflammatory response associated with viral vectors. Other factors that have hindered progress are related to lung structure, including significant barrier generated by tight junctions and the presence of apical mucus. Thus, achieving and maintaining suitable expression of exogenous genes in respiratory tissues remains a challenging endeavor, and there is a need in the field for therapeutic vectors having improved expression in respiratory tissues.

### SUMMARY

Provided herein are nucleic acid vectors having regulatory elements that confer improved transgene expression (e.g., improved expression levels and/or persistence) in target cells, such as airway cells (e.g., airway epithelial cells).

In one aspect, the provided herein is a circular DNA vector comprising a promoter, a transgene, and a scaffold matrix attachment region (S/MAR) sequence, wherein the transgene is a therapeutic protein-encoding sequence derived from a protein expressed in respiratory epithelium, and wherein the circular DNA vector lacks a drug resistance gene. In some embodiments, the therapeutic protein is a cystic fibrosis transmembrane receptor (CFTR), such as a human CFTR. In some embodiments, the CFTR is at least 95% identical to SEQ ID NO: 1. In some embodiments, the CFTR comprises SEQ ID NO: 1. In some embodiments, the S/MAR sequence is an interferon-beta S/MAR sequence or a functional variant thereof (e.g., an interferon-beta S/MAR sequence or a functional variant thereof that comprises SEQ ID NO: 2). In some embodiments, the promoter is an elongation factor 1 alpha (EF1A) promoter.

In some embodiments, the promoter is operably linked 5′ to the transgene, and the S/MAR sequence is operably linked 3′ to the transgene. In some embodiments, the circular DNA vector further lacks a recombination site, (e.g., the circular DNA vector may be a synthetic circular DNA vector made in a cell-free process).

In some embodiments, the circular DNA vector is between 7 and 9 kbp in length.

In some embodiments, the 3′ end of the S/MAR sequence is connected to the 5′ end of the promoter by a non-bacterial sequence of less than 500 bp. In some embodiments, the non-bacterial sequence connecting the 3′ end of the S/MAR sequence to the 5′ end of the promoter is less than 100 bp. In some embodiments, the non-bacterial sequence connecting the 3′ end of the S/MAR sequence to the 5′ end of the promoter corresponds to a restriction enzyme cut site overhang (e.g., a type IIs restriction enzyme cute site overhang). In some embodiments, the non-bacterial sequence connecting the 3′ end of the S/MAR sequence to the 5′ end of the promoter comprises SEQ ID NO: 3. In some embodiments, the non-bacterial sequence connecting the 3′ end of the S/MAR sequence to the 5′ end of the promoter comprises SEQ ID NO: 4.

In another aspect, the provided herein is a nucleic acid vector (e.g., circular DNA vector, e.g., synthetic circular DNA vector) comprising a hypersensitivity sequence operably linked 5′ to a transgene, wherein the hypersensitivity sequence is derived from human chromosome 7, position 117,174,974-117,076,392 of Genome Reference Consortium Human Build 37 release 19 (GRch37/hg19) (e.g., the hypersensitivity sequence comprises SEQ ID NO: 5, or a functional variant thereof.) In some embodiments, the transgene is a therapeutic protein-encoding sequence derived from a protein expressed in respiratory epithelium. In some embodiments, the therapeutic protein is a cystic fibrosis transmembrane receptor (CFTR). In some embodiments, the CFTR is a human CFTR. In some embodiments, the CFTR is at least 95% identical to SEQ ID NO: 1. In some embodiments, the CFTR comprises SEQ ID NO: 1.

In some embodiments, the hypersensitivity sequence is at least 95% identical to SEQ ID NO: 5. In some embodiments, the hypersensitivity sequence comprises SEQ ID NO: 5.

In some embodiments, the nucleic acid vector further comprising a promoter operably linked 5′ to the transgene, e.g., wherein the promoter is operably linked 3′ to the hypersensitivity sequence. In some embodiments, the promoter comprises SEQ ID NO: 6, or a functional variant thereof. In some embodiments, the promoter is at least 95% identical to SEQ ID NO: 6 or SEQ ID NO: 7. In some embodiments, the promoter comprises SEQ ID NO: 7. In some embodiments, the promoter comprises SEQ ID NO: 6.

In some embodiments, the transgene is operatively linked to a reporter sequence.

In some embodiments, the nucleic acid vector is a non-integrating DNA vector, such as a plasmid DNA vector, a minicircle DNA vector, a nanoplasmid DNA vector, a synthetic circular DNA vector, a closed-ended ONA vector, a doggybone DNA vector, or a ministring DNA vector. In some embodiments, the nucleic acid vector is a circular DNA vector that lacks a drug resistance gene. In some embodiments, the circular DNA vector further lacks a recombination site. For example, in some embodiments, the nucleic acid vector is a synthetic circular DNA vector.

In some embodiments, the nucleic acid vector (e.g., circular DNA vector, e.g., synthetic circular DNA vector) is between 7 and 9 kbp in length.

In some embodiments, the 3′ end of the transgene is connected to the 5′ end of the hypersensitivity sequence by a non-bacterial sequence of less than 500 bp. In some embodiments, the non-bacterial sequence connecting the 3′ end of the transgene to the 5′ end of the hypersensitivity sequence is less than 100 bp. In some embodiments, the non-bacterial sequence connecting the 3′ end of the transgene to the 5′ end of the hypersensitivity sequence corresponds to a restriction enzyme cut site overhang.

In another aspect, the invention provides a pharmaceutical composition comprising a therapeutically effective amount of the vector of any one of the preceding aspects or embodiments thereof, and a pharmaceutically acceptable carrier. In some embodiments, the circular DNA vector or nucleic acid vector is nonviral, e.g., naked. In other embodiments, the nucleic acid vector (e.g., circular DNA vector) is formulated as a liposomal or nanoparticulate formulation. In some embodiments, the nucleic acid vector (e.g., circular DNA vector) is formulated for respiratory delivery.

### BRIEF DESCRIPTION OF THE DRAWINGS

FIGS. 1A and 1B are maps showing the arrangement of elements on nucleic acid vectors that encode CFTR and contain a scaffold/matrix attachment region (S/MAR). FIG. 1A shows a plasmid (p-1731; SEQ ID NO: 14), which contains backbone elements (bacterial origin of replication and drug resistance gene). FIG. 1B shows a synthetic circular DNA vector (C³DNA; C³-1731; SEQ ID NO: 15), in which the backbone elements have been removed using a cell-free process.

FIGS. 2A and 28 are maps showing the arrangement of elements on nucleic acid vectors that encode CFTR and contain an upstream hypersensitivity sequence (HS). FIG. 2A shows a plasmid (p-1732; SEQ ID NO: 16), which contains backbone elements (bacterial origin of replication and drug resistance gene). FIG. 2B shows a synthetic circular DNA vector (C³DNA; C³-1732; SEQ ID NO: 17), in which the backbone elements have been removed using a cell-free process.

FIGS. 3A and 38 are schematic illustrations showing a linear version of the vectors shown in FIGS. 1 and 2, respectively.

FIG. 4 is a photograph of a Western blot gel showing CFTR protein expressed by HEK293T cells transfected with p-1731 and p-1732.

FIG. 5 is a fluorescent image of a Western blot gel showing CFTR protein expressed by HEK293T cells transfected with p-1732. C3-1732, and p-1731.

FIG. 6 is a photograph of a Western blot gel showing CFTR protein expressed by HEK293T cells transfected with p-1731 and C³-1731.

FIG. 7 is a graph showing expression levels of GFP encoded by a synthetic circular DNA vector (c3DNA) compared to GFP encoded by plasmid DNA vector (P1003) in suspended primary human bronchial epithelial cells across a range of electrotransfer conditions (applied voltage, duration of each pulse (ms), and number of pulses).

### DETAILED DESCRIPTION

The present invention provides constructs for improved expression of transgenes (e.g., respiratory transgenes for expression in the airway), nucleic acid vectors thereof, pharmaceutical compositions thereof, and methods of use thereof (e.g., methods of treatment). The invention is based, at least in part, on the development of regulatory elements that affect transgene expression and/or persistence of expression constructs that include the regulatory elements. Regulatory elements of the expression constructs can include hypersensitivity sequences, promoters, scaffold/matrix attachment region (S/MAR) sequences, enhancers, insulators, and other regulatory elements that can enhance expression and/or persistence in host cells. Expression constructs, nucleic acid vectors and pharmaceutical compositions thereof, and methods of use thereof, disclosed herein can provide effective, durable treatments for respiratory diseases.

### I. Definitions

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. In the event of any conflicting definitions between those set forth herein and those of a referenced publication, the definition provided herein shall control.

As used herein, the term “expression construct” refers to a nucleic acid sequence (e.g., DNA sequence) that is expressed by a cell upon delivery to the cell, e.g., by a nucleic acid vector containing the expression construct. An expression construct may include a sequence of interest (e.g., one or more transgenes, e.g., therapeutic transgenes) and regulatory elements operably linked thereto (e.g., hypersensitivity sites, promoters, S/MARs, intronic sequences, insulators, etc.) which can enhance expression and/or persistence of the DNA vector in a target cell.

As used herein, the terms “vector” and “nucleic acid vector” are used interchangeably and refer to a nucleic acid molecule capable of delivering a therapeutic sequence to which is it linked into a target cell in which the therapeutic sequence can then be transcribed, replicated, processed, and/or expressed in the target cell. After a target cell or host cell processes the therapeutic sequence of the vector, the therapeutic sequence is not considered a vector. One type of vector is a “plasmid”, which refers to a circular double stranded DNA loop containing a bacterial backbone into which additional DNA segments may be ligated. Another type of vector is a phage vector. Another type of vector is a viral vector (e.g., adeno-associated viral (AAV) vector), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors” or “expression vectors”). Any of the nucleic acid vectors described herein may be referred to as “isolated nucleic acid vectors.”

As used herein, the term “circular DNA vector” refers to a DNA vector in a circular form. Such circular form is typically capable of being amplified into concatemers by rolling circle amplification. A linear double-stranded nucleic acid having conjoined strands at its termini (e.g., covalently conjugated backbones, e.g., by hairpin loops or other structures) is not a circular vector, as used herein. The term “circular DNA vector” is used interchangeably herein with the terms “covalently closed and circular DNA vector” and “C³DNA.” A skilled artisan will understand that such circular vectors include vectors that are covalently closed with supercoiling and complex DNA topology, as is described herein. In particular embodiments, a circular DNA vector is supercoiled (e.g., monomeric supercoiled). In other embodiments, a circular DNA vector is relaxed open circular (covalently closed without supercoiling). In certain instances, a circular DNA vector lacks a bacterial origin of replication.

The term “synthetic,” as used herein, describes a vector (e.g., a circular DNA vector) that was produced in a cell-free process in which bacterial cells are absent from their production from templates. Exemplary cell-free processes for producing synthetic circular DNA vectors are provided in Examples 1 and 2 and in International Patent Publication WO 2019/178500, which is incorporated herein by reference in its entirety.

As used herein, the term “therapeutic sequence” refers to the portion of a DNA molecule (e.g., a DNA vector (e.g., plasmid DNA vector or a concatemer thereof) that contains any genetic material required for transcription in a target cell of one or more therapeutic moieties, which may include one or more coding sequences, promoters, terminators, introns, and/or other regulatory elements (e.g., S/MARS and/or hypersensitivity sequences). A therapeutic moiety can be a therapeutic protein (e.g., a replacement protein (e.g., a protein that replaces a defective protein in the target cell) and/or a therapeutic nucleic acid (e.g., one or more microRNAs). In DNA vectors having more than one transcription unit, the therapeutic sequence contains the plurality of transcription units. A therapeutic sequence may include one or more genes (e.g., heterologous genes or transgenes, e.g., respiratory genes) to be administered for a therapeutic purpose. In some embodiments, the therapeutic sequence is a mammalian sequence (e.g., a human sequence).

As used herein, an “respiratory gene” means a gene that is preferentially, selectively, or exclusively expressed in airway tissue or that is involved in respiratory functions, such as breathing.

As used herein, the term “protein” refers to a plurality of amino acids attached to one another through peptide bonds (i.e., as a primary structure), including multimeric (e.g., dimeric, trimeric, etc.) proteins that are non-covalently associated (e.g., proteins having quaternary structure). Thus, the term “protein” encompasses peptides (e.g., polypeptides), native proteins, recombinant proteins, and fragments thereof. In some embodiments, a protein has a primary structure and no secondary, tertiary, of quaternary structure in physiological conditions. In some embodiments, a protein has a primary and secondary structure and no tertiary or quaternary structure in physiological conditions. In particular embodiments, a protein has a primary structure, a secondary structure, and a tertiary structure, but no quaternary structure in physiological conditions (e.g., a monomeric protein having one or more folded alpha-helices and/or beta sheets). In some embodiments, any of the proteins described herein have a length of at least 25 amino acids (e.g., 50 to 1,000 amino acids).

As used herein, the term “therapeutic protein” refers to a protein that can treat a disease or disorder in a subject. In some embodiments, a therapeutic protein is a therapeutic replacement protein administered to replace a defective (e.g., mutated) protein in a subject. In some embodiments, a therapeutic protein is the same or functionally similar to a native protein that is not defective in a subject.

As used herein, the term “therapeutic replacement protein” refers to a protein that is structurally similar to (e.g., structurally identical to) a protein that is endogenously expressed by a normal (e.g., healthy) individual. A therapeutic replacement protein can be administered to an individual that suffers from a disorder associated with a dysfunction of (or lack of) the protein to be replaced. In some embodiments, the therapeutic replacement protein corrects a defect in a protein resulting from a mutation (e.g., a point mutation, an insertion mutation, a deletion mutation, or a splice variant mutation) in the gene encoding the protein. Therapeutic replacement proteins do not include non-endogenous proteins, such as proteins associated with a pathogen (e.g., as part of a vaccine). Therapeutic replacement proteins may include enzymes, growth factors, hormones, interleukins, interferons, cytokines, anti-apoptosis factors, anti-diabetic factors, coagulation factors, anti-tumor factors, liver-secreted proteins, or neuroprotective factors. In some instances, the therapeutic replacement protein is monogenic.

As used herein, the term “backbone sequence” refers to a portion of plasmid DNA outside the therapeutic sequence that includes one or more bacterial origins of replication or fragments thereof, one or more drug resistance genes or fragments thereof, one or more recombination sites, or any combination thereof. In some embodiments, the backbone sequence includes one or more bacterial origins of replication. Backbone sequences include truncated plasmid backbones of 20 base pairs or more (e.g., 31-40, e.g., 38 base pairs), which may include, e.g., a functional origin of replication.

As used herein, the term “recombination site” refers to a nucleic acid sequence that is a product of site-specific recombination, which includes a first sequence that corresponds to a portion of a first recombinase attachment site and a second sequence that corresponds to a portion of a second recombinase attachment site. One example of a hybrid recombination site is attR, which is a product of site-specific recombination and includes a first sequence that corresponds to a portion of attP and a second sequence that corresponds to a portion of attB. Alternatively, recombination sites can be generated from Cre/Lox recombination. Thus, a vector generated from Cre/Lox recombination (e.g., a vector including a LoxP site) includes a recombination site, as used herein. Other site-specific recombination events that generate recombination sites involve, e.g., lambda integrase, FLP recombinase, and Kw recombinase. Nucleic acid sequences that result from non-site-specific recombination events (e.g., ITR-mediated intermolecular recombination) are not recombination sites, as defined herein.

As used herein, the term “flank,” “flanking,” and “flanked” refer to a pair of regions or points on a nucleic acid molecule (e.g., a plasmid DNA vector) that are outside a reference region of the nucleic acid molecule. In some embodiments, a pair of regions or points flanking a reference region on a nucleic acid are adjacent to (i.e., abut) the reference region (i.e., there are no intervening bases between the reference point and the flanking point). In other embodiments, a pair of regions or points on a nucleic acid molecule that flank a reference region are separated from the reference region by one or more intervening bases (e.g., up to 1,000 intervening bases).

As used herein, the term “operably linked” refers to an arrangement of elements, wherein the components so described are configured so as to perform their usual function. A nucleic acid is “operably linked” when it is placed into a functional relationship with another nucleic acid sequence. For example, a promoter is operably linked to one or more heterologous genes if it affects the transcription of the one or more heterologous genes. Further, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence, and the promoter sequence can still be considered “operably linked” to the coding sequence.

As used herein, the terms “scaffold/matrix attachment region” and “S/MAR” each refers to a DNA sequence of at least 200 nucleotides which mediates attachment of the DNA to a nuclear matrix of a eukaryotic cell, wherein the DNA sequence has at least three sequence motifs ATTA per 100 nucleotides over a stretch of at most 200 nucleotides. Exemplary S/MAR sequences are described in Liebich et al., Nucleic Acids Res. 2002, 30:312-374 and in International Patent Publication No. WO 2019/060253, the S/MAR descriptions of each of which are incorporated herein by reference.

As used herein, the term “cystic fibrosis transmembrane regulator (CFTR)” refers to any native CFTR from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, splice variants, and fragments thereof. Functionally equivalent and Improved variants can be determined on the basis of known CFTR signaling (e.g., ion and water secretion and absorption in epithelial tissues). CFTR encompasses full-length, unprocessed CFTR, as well as any form of CFTR that results from native processing in the cell. An exemplary human CFTR sequence is provided as National Center for Biotechnology Information (NCBI) Gene ID: 1080. In some instances, a CFTR transgene is encoded by a sequence having at least 95% sequence identity to any one of SEQ ID NO: 1 (e.g., at least 96%, at least 97%, at least 98%, at least 99% or 100% sequence identity to SEQ ID NO: 1).

The terms “regulatory element” and “control element” are used interchangeably herein and each refer to a non-coding nucleic acid region, such as a promoter, enhancer, and silencer, which function to affect gene expression (e.g., level of expression and/or persistence of expression). In some embodiments, a regulatory element is not transcribed into mRNA. In other embodiments, a regulatory element is transcribed into mRNA but not translated into protein. Suitable regulatory elements are described in International Publication No. WO 2021/055760, which is incorporated herein by reference in its entirety.

A regulatory element is “derived” from a reference sequence (e.g., a native intron) when it contains a functional sequence, or functional variant of a sequence, contained within the reference sequence (e.g., a functional sequence, or functional variant of a sequence, having at least 20, at least 30, at least 40, at least 50, at least 100, at least 150, at least 200, at least 300, at least 400, or at least 500 nucleotide bases having at least 90%, 95%, 96%, 97%, 98%, 99%, or 100% sequence identity with the reference sequence). A regulatory element derived from a reference sequence need not have the same level of function or type of function as the reference sequence; the functional sequence of the regulatory element must confer a detectable function (e.g., improve the level and/or persistence of expression, compared to an expression construct lacking the functional sequence of the regulatory element).

The term “promoter” refers to a regulatory element that regulates transcription of a gene operably linked thereto and includes (a) one or more sequence sufficient to direct transcription and/or (b) recognition sites for RNA polymerase and other transcription factors required for efficient transcription. In some embodiments, the promoter is operably linked 5′ to the gene (e.g., operably linked upstream of the gene). Some promoters can direct cell-specific expression.

As used herein, the term “naked” refers to a nucleic acid molecule (e.g., a circular DNA vector) that is not encapsulated in a lipid envelope (e.g., a liposome) or a polymer matrix and is not physically associated with (e.g., covalently or non-covalently bound to) a solid structure (e.g., a particulate structure) upon administration to the individual. In some instances of the present invention, a pharmaceutical composition includes a naked circular DNA vector.

As used herein, the term “isolated” means artificially produced and not integrated into a native host genome. For example, isolated nucleic acid vectors include nucleic acid vectors that are naked, as well as those that are encapsulated in a lipid envelope (e.g., a liposome) or a polymer matrix. In some embodiments, the term “isolated” refers to a DNA vector that is: (i) synthetic, e.g., amplified in vitro (e.g., in a cell-free environment), for example, by rolling-circle amplification or polymerase chain reaction (PCR); (ii) recombinantly produced by molecular cloning; (Iii) purified, as by restriction endonuclease cleavage and gel electrophoretic fractionation, or column chromatography; or (iv) synthesized by, for example, chemical synthesis. An Isolated nucleic acid vector is one which is readily manipulable by recombinant DNA techniques well-known in the art. Thus, a nucleotide sequence contained in a vector in which 5′ and 3′ restriction sites are known or for which PCR primer sequences have been disclosed is considered isolated, but a nucleic acid sequence existing in its native state in its natural host is not. An Isolated nucleic acid vector may be substantially purified, but need not be.

As used herein, the terms “individual” and “subject” are used interchangeably and include any mammal in need of treatment or prophylaxis, e.g., by a therapeutic circular DNA vector, or pharmaceutical composition thereof, described herein. In some embodiments, the individual or subject is a human. In other embodiments, the individual or subject is a non-human mammal (e.g., a non-human primate (e.g., a monkey), a mouse, a pig, a rabbit, a cat, or a dog). The individual or subject may be male or female.

As used herein, an “effective amount” or “effective dose” of a nucleic acid vector, or pharmaceutical composition thereof, refers to an amount sufficient to achieve a desired biological, pharmacological, or therapeutic effect, e.g., when administered to the individual according to a selected administration form, route, and/or schedule. As will be appreciated by those of ordinary skill in this art, the absolute amount of a particular composition that is effective can vary depending on such factors as the desired biological or pharmacological endpoint, the agent to be delivered, the target tissue, etc. Those of ordinary skill in the art will further understand that an “effective amount” can be contacted with cells or administered to a subject in a single dose or through use of multiple doses. An effective amount of a composition to treat a disease may slow or stop disease progression or increase partial or complete response, relative to a reference population, e.g., an untreated or placebo population, or a population receiving the standard of care treatment.

As used herein, “treatment” (and grammatical variations thereof such as “treat” or “treating”) refers to clinical intervention in an attempt to alter the natural course of the individual being treated, which can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and improved prognosis. In some embodiments, therapeutic circular DNA vectors of the invention are used to delay development of a disease or to slow the progression of a disease (e.g., respiratory disease, such as cystic fibrosis).

As used herein, a “target cell” refers to a cell that expresses a therapeutic protein encoded by a therapeutic gene. In some embodiments, a target cell is a respiratory cell or an airway cell. For example, in particular embodiments, a target cell is an airway epithelial cell (e.g., a lung epithelial cell, a nasal epithelial cell, a tracheal epithelial cell, bronchial epithelial cell, bronchiolar epithelial cell, or an alveolar epithelial cell).

As used herein, “delivering,” “to deliver,” and grammatical variations thereof, means causing an agent (e.g., a DNA vector) to access a target cell. The agent can be delivered by administration of the agent to an individual having the target cell (e.g., systemically or locally administering the agent) such that the agent gains access to the organ or tissue in which the target cell resides (e.g., airway). Additionally. or alternatively, the agent can be delivered by applying a stimulus to a tissue or organ harboring the agent, wherein the stimulus causes the agent to enter the target cell. Thus, in some instances, an agent is delivered to a target cell by transmitting an electric field into a tissue harboring the agent at conditions suitable for electrotransfer of the agent into a target cell within the tissue.

As used herein, “administering” is meant a method of giving a dosage of an agent (e.g., a DNA vector) of the invention or a composition thereof (e.g., a pharmaceutical composition, e.g., a pharmaceutical composition including a DNA vector) to an individual. The compositions utilized in the methods described herein can be administered locally to the airway, e.g., by flood, spray, of aerosolization of the agent.

As used herein, “electrotransfer” refers to movement of a molecule (e.g., a nucleic acid, e.g., a naked nucleic acid) across a membrane of a target cell (e.g., from outside to inside the target cell) that is caused by transmission of an electric field (e.g., a pulsed electric field) to the microenvironment in which the cell resides. Electrotransfer may occur as a result of electrophoresis, i.e., movement of a molecule (e.g., a nucleic acid, e.g., a naked nucleic acid) along an electric field (e.g., in the direction of current), based on a charge of the molecule. Electrophoresis can induce electrotransfer, for example, by moving a molecule (e.g., a nucleic acid, e.g., a naked nucleic acid) into proximity of a cell membrane to allow a biotransport process (e.g., endocytosis including pinocytosis or phagocytosis) or passive transport (e.g., diffusion or lipid partitioning) to carry the molecule into the cell. Additionally, or alternatively, electrotransfer may occur as a result of electroporation, i.e., generation of pores in the target cell caused by transmission of an electric field (e.g., a pulsed electric field), wherein the size, shape, and duration of the pores are suitable to accommodate movement of a molecule (e.g., a nucleic acid, e.g., a naked nucleic acid) from outside the target cell to inside the target cell. Thus, in some instances, electrotransfer occurs as a result of a combination of electrophoresis and electroporation.

The terms “level of expression” or “expression level” are used interchangeably and generally refer to the amount of a polynucleotide or an amino acid product or protein in a biological sample (e.g., airway epithelium). “Expression” generally refers to the process by which gene-encoded information is converted into the structures present and operating in the cell. Therefore, according to the invention, “expression” may refer to transcription into a polynucleotide, translation into a protein, or post-translational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

As used herein, the term “expression persistence” refers to the duration of time during which a therapeutic sequence, or a functional portion thereof (e.g., one or more coding sequences of a therapeutic DNA vector), is expressible in the cell in which it was transfected (“intra-cellular persistence”) or any progeny of the cell in which it was transfected (“trans-generational persistence”). A therapeutic sequence, or functional portion thereof, may be expressible if it is not silenced, e.g., by DNA methylation and/or histone methylation and compaction. Expression persistence can be assessed by detecting or quantifying (i) mRNA transcribed from the therapeutic sequence in the target cell or progeny thereof (e.g., through qPCR, RNA-seq, or any other suitable method) and (ii) protein translated from the therapeutic sequence in the target cell or progeny thereof (e.g., through Western blot, ELISA, or any other suitable method). In some instances, expression persistence is assessed by detecting or quantifying therapeutic DNA in the target cell or progeny thereof (e.g., the presence of therapeutic circular DNA vector in the target cell, e.g., through episomal DNA copy number analysis) in conjunction with either or both of (i) mRNA transcribed from the therapeutic sequence in the target cell or progeny thereof and (ii) protein translated from the therapeutic sequence in the target cell or progeny thereof. Expression persistence of a therapeutic sequence, or a functional portion thereof, can be quantified relative to a reference vector, such as a control vector produced in bacteria (e.g., a circular vector produced in bacteria or having one or more bacterial signatures not present in the vector of the invention (e.g., a plasmid), using any gene expression characterization method known in the art. Expression persistence can be quantified at any given time point following administration of the vector. For example, in some embodiments, expression of a DNA vector of the invention persists for at least two weeks after administration if it is detectable in the target cell, or progeny thereof, two weeks after administration of the DNA vector. In some embodiments, expression of a DNA vector “persists” in a target cell if it is detectable in the target cell at one week, two weeks, three weeks, four weeks, six weeks, two months, three months, four months, five months, six months, seven months, eight months, nine months, ten months, eleven months, one year, or longer after administration. In some embodiments, expression of a DNA vector is said to persist for a given period after administration if any detectable fraction of the original expression level remains (e.g., at least 1%, at least 5%, at least 10%, at least 20%, at least 30%, at least 40%, at least 50%, at least 70%, or at least 100% of the original expression level) after the given period of time (e.g., one week, two weeks, three weeks, four weeks, six weeks, two months, three months, four months, five months, six months, seven months, eight months, nine months, ten months, eleven months, one year, or longer after administration).

As used herein, “intra-cellular persistence” refers to the duration of time during which a therapeutic sequence, or a functional portion thereof (e.g., one or more coding sequences of a therapeutic ONA vector), is expressible in the cell in which it was transfected (e.g., a target cell, such as a post-mitotic cell or a quiescent cell). Intra-cellular persistence can be assessed by detecting or quantifying (i) MRNA transcribed from the therapeutic sequence in the target cell and (ii) protein translated from the therapeutic sequence in the target cell. In some instances, intra-cellular persistence is assessed by detecting or quantifying therapeutic DNA in the target cell (e.g., the presence of DNA vector in the target cell) in conjunction with either or both of (i) mRNA transcribed from the therapeutic sequence in the target cell and (ii) protein translated from the therapeutic sequence in the target cell. In some embodiments, a ONA vector of the invention exhibits improved intra-cellular persistence relative to a reference vector (e.g., a DNA vector containing a regulatory element exhibits improved intra-cellular persistence relative to a reference vector that does not contain the regulatory element; or a synthetic circular DNA vector exhibits improved intra-cellular persistence relative to a reference vector that is not synthetic, e.g., a plasmid DNA vector)

As used herein, “trans-generational persistence” refers to the duration of time during which a therapeutic sequence, or a functional portion thereof (e.g., one or more coding sequences of a therapeutic DNA vector), is expressible in progeny of the cell in which the gene was transfected (e.g., progeny of the target cell, such as first-generation, second-generation, third-generation, or fourth-generation descendants of the cell in which the gene was transfected, e.g., through a therapeutic circular DNA vector). Trans-generational persistence accounts for any dilution of a gene over cell divisions and may therefore be useful in measuring persistence of a vector in a dividing tissue over lime. In some embodiments, the therapeutic circular DNA vector of the invention exhibits improved trans-generational persistence relative to a reference vector (e.g., a plasmid DNA vector). Trans-generational persistence can be assessed by detecting or quantifying (i) mRNA transcribed from the therapeutic sequence in progeny of the target cell and (ii) protein translated from the therapeutic sequence in progeny of the target cell. In some instances, intra-cellular persistence is assessed by detecting or quantifying therapeutic DNA in progeny of the target cell (e.g., the presence of therapeutic circular DNA vector in progeny of the target cell) in conjunction with either or both of (i) mRNA transcribed from the therapeutic sequence in progeny of the target cell and (ii) protein translated from the therapeutic sequence in progeny of the target cell. In some embodiments, the DNA vector of the invention exhibits improved trans-generational persistence relative to a reference vector (e.g., a DNA vector containing a regulatory element exhibits improved trans-generational persistence relative to a reference vector that does not contain the regulatory element; or a synthetic circular DNA vector exhibits improved trans-generational persistence relative to a reference vector that is not synthetic, e.g., a plasmid DNA vector).

As used herein, a “functional variant” of a nucleic acid sequence differs in at least one nucleic acid residue from the reference nucleic acid sequence, such as a naturally occurring nucleic acid sequence, wherein relevant functional activity of the variant is at least 90% of the level of relevant functional activity of the reference nucleic acid sequence (e.g., substantially similar to the relevant function of the reference nucleic acid sequence). In this context, the difference in at least one nucleic acid residue may consist, for example, in a mutation of an nucleic acid residue to another nucleic acid, a deletion or an insertion. A variant may encode a homolog, isoform, or transcript variant of a therapeutic protein or a fragment thereof encoded by the reference nucleic acid sequence, wherein the homolog, isoform or transcript variant is characterized by a degree of identity or homology, respectively, as defined herein.

In some instances, a functional variant of a polynucleotide or polypeptide includes at least one nucleic acid substitution (e.g., 1-100 nucleic acid or amino acid substitutions, 1-50 nucleic acid or amino acid substitutions, 1-20 nucleic acid or amino acid substitutions, 1-10 nucleic acid or amino acid substitutions, e.g., 1 nucleic acid or amino acid substitution, 2 nucleic acid or amino acid substitutions, 3 nucleic acid or amino acid substitutions, 4 nucleic acid or amino acid substitutions, 5 nucleic acid or amino acid substitutions, 6 nucleic acid or amino acid substitutions, 7 nucleic acid or amino acid substitutions, 8 nucleic acid or amino acid substitutions, 9 nucleic acid or amino acid substitutions, or 10 nucleic acid or amino acid substitutions). Nucleic acid substitutions that result in the expressed polypeptide having an exchanged in amino acids from the same class are referred to herein as conservative substitutions. In particular, these are amino acids having aliphatic side chains, positively or negatively charged side chains, aromatic groups in the side chains or amino acids, the side chains of which can form hydrogen bridges, e.g., side chains which have a hydroxyl function. By conservative substitution, e.g., an amino acid having a polar side chain may be replaced by another amino acid having a corresponding polar side chain, or, for example, an amino acid characterized by a hydrophobic side chain may be substituted by another amino acid having a corresponding hydrophobic side chain (e.g., serine (threonine) by threonine (serine) or leucine (isoleucine) by isoleucine (leucine)).

In order to determine the percentage to which two sequences (e.g., nucleic acid sequences, e.g., DNA or amino acid sequences) are identical, the sequences can be aligned in order to be subsequently compared to one another. For this purpose, gaps can be inserted into the sequence of the first sequence and the component at the corresponding position of the second sequence can be compared. If a position in the first sequence is occupied by the same component as is the case at a corresponding position in the second sequence, the two sequences are identical at this position. The percentage, to which two sequences are identical, is a function of the number of identical positions divided by the total number of positions. The percentage to which two sequences are identical can be determined using a mathematical algorithm. A preferred, but not limiting, example of a mathematical algorithm, which can be used is the algorithm of Karlin et al. (1993), PNAS USA, 90:5873-5877 or Altschul et al. (1997), Nucleic Acids Res., 25:3389-3402. Such an algorithm can be integrated, for example, in the BLAST program. Sequences which are identical to the sequences of the present invention to a certain extent can be identified by this program.

The term “pharmaceutically acceptable” means safe for administration to a mammal, such as a human. In some embodiments, a pharmaceutically acceptable composition is approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which a vector or composition of the invention is administered. Examples of suitable pharmaceutical carriers are described in “Remington's Pharmaceutical Sciences,” Mack Publishing Co., Easton, PA., 23^{rd} edition, 2020.

The terms “a” and “an” mean “one or more of.” For example, “a gene” is understood to represent one or more such genes. As such, the terms “a” and “an,” “one or more of a (or an),” and “at least one of a (or an)” are used interchangeably herein.

As used herein, the term “about” refers to a value within +10% variability from the reference value, unless otherwise specified.

The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B. and C.” The term “or” can be used conjunctively or disjunctively unless the context specifically refers to a disjunctive use.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the present disclosure, subject to any specifically excluded limit in the stated range. Where the stated range includes one or both of the limits, ranges excluding either or both of those included limits are also included in the present disclosure, unless the context clearly dictates otherwise.

As used in this specification and claim(s), the words “comprising” (and any form of comprising, such as “comprise” and “comprises”), “having” (and any form of having, such as “have” and “has”). “including” (and any form of including, such as “includes” and “include”) or “containing” (and any form of containing, such as “contains” and “contain”) are inclusive or open-ended and do not exclude additional, unrecited elements or method steps, it is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Certain specific details of this description are set forth in order to provide a thorough understanding of various embodiments. However, one skilled in the art will understand that the present disclosure may be practiced without these details. In other instances, well-known techniques or methods have not been shown or described in detail to avoid unnecessarily obscuring descriptions of the embodiments. Further, headings provided herein are for convenience only and do not limit the scope or meaning of the claimed disclosure.

Although methods and materials similar or equivalent to those described herein can be used in the practice or testing of the present disclosure, suitable methods, and materials are described below.

For any conflict in definitions between various sources or references, the definition provided herein shall control.

### II. Expression Constructs

Embodiments disclosed herein include expression constructs that provide for expression of a transgene, such as a therapeutic sequence, by a nucleic acid vector (e.g., a nonviral DNA vector (e.g., a naked DNA vector), a circular DNA vector (e.g., a supercoiled circular DNA vector), and/or a synthetic DNA vector (e.g., a synthetic circular DNA vector). Various elements can be included in such expression constructs, such as therapeutic genes, promoters, and regulatory elements that can enhance expression and/or persistence of the DNA vector in a target cell (e.g., a retinal cell). In some of the embodiments described herein, the coding sequence or regulatory elements may be human sequences or a human-derived sequences. Nucleic acid vectors of the invention can include any of the expression constructs described herein, or combination thereof.

### A. Coding Sequences and Proteins

Some embodiments of expression constructs disclosed herein include one or more coding sequences for one or more genes, for example, respiratory genes, such as a CFTR-encoding gene. In some embodiments, the respiratory gene (e.g., CFTR-encoding gene) is a gene that is expressed in airway tissue, such as, an airway epithelium (e.g., a lung epithelium, a nasal epithelium, a tracheal epithelium, bronchial epithelium, bronchiole, or alveolus). In some embodiments, the respiratory gene encodes for a therapeutic protein, such as CFTR.

Coding sequences included in expression constructs (e.g., CFTR-encoding expression constructs) can include genes that are involved in respiratory diseases, such as cystic fibrosis. In some embodiments, the coding sequence is a cDNA of CFTR. In some embodiments, the coding sequence is a codon-optimized CFTR sequence. In some instances, the coding sequence is a functional variant of the respiratory gene, e.g., a CFTR-encoding gene. In some embodiments, the coding sequence of CFTR is, or comprises, the nucleic acid sequence of SEQ ID NO: 1. In some embodiments, the coding sequence of CFTR includes a sequence having at least 75, 80, 85, 90, 95, 98, 99, 99.5, or 99.9% sequence identity to SEQ ID NO: 1. In some embodiments, the coding sequence may be a human sequence or a human-derived sequence.

In some embodiments, the genes and/or coding sequences included in expression constructs and nucleic acid vectors described herein are greater than 4.5 Kb in length (e.g., one or more coding sequences, together or each alone, are from 4.5 Kb to 25 Kb, from 4.6 Kb to 24 Kb, from 4.7 Kb to 23 Kb, from 4.8 Kb to 22 Kb, from 4.9 Kb to 21 Kb, from 5.0 Kb to 20 Kb, from 5.5 Kb to 18 Kb, from 6.0 Kb to 17 Kb, from 6.5 Kb to 16 Kb, from 7.0 Kb to 15 Kb, from 7.5 Kb to 14 Kb, from 8.0 Kb to 13 Kb, from 8.5 Kb to 12.5 Kb, from 9.0 Kb to 12.0 Kb, from 9.5 Kb to 11.5 Kb, or from 10.0 Kb to 11.0 Kb in length, e.g., from 4.5 Kb to 8 Kb, from 8 Kb to 10 Kb, from 10 Kb to 15 Kb, from 15 Kb to 20 Kb in length, or greater, e.g., from 4.5 Kb to 5.0 Kb, from 5.0 Kb to 5.5 Kb, from 5.5 Kb to 6.0 Kb, from 6.0 Kb to 6.5 Kb, from 6.5 Kb to 7.0 Kb, from 7.0 Kb to 7.5 Kb, from 7.5 Kb to 8.0 Kb, from 8.0 Kb to 8.5 Kb, from 8.5 Kb to 9.0 Kb, from 9.0 Kb to 9.5 Kb, from 9.5 Kb to 10 Kb, from 10 Kb to 10.5 Kb, from 10.5 Kb to 11 Kb, from 11 Kb to 11.5 Kb, from 11.5 Kb to 12 Kb, from 12 Kb to 12.5 Kb, from 12.5 Kb to 13 Kb, from 13 Kb to 13.5 Kb, from 13.5 Kb to 14 Kb, from 14 Kb to 14.5 Kb, from 14.5 Kb to 15 Kb, from 15 Kb to 15.5 Kb, from 15.5 Kb to 16 Kb, from 16 Kb to 16.5 Kb, from 16.5 Kb to 17 Kb, from 17 Kb to 17.5 Kb, from 17.5 Kb to 18 Kb, from 18 Kb to 18.5 Kb, from 18.5 Kb to 19 Kb, from 19 Kb to 19.5 Kb, from 19.5 Kb to 20 Kb, from 20 Kb to 21 Kb, from 21 Kb to 22 Kb, from 22 Kb to 23 Kb, from 23 Kb to 24 Kb, from 24 Kb to 25 Kb in length, or greater, e.g., about 4.5 Kb, about 5.0 Kb, about 5.5 Kb, about 6.0 Kb, about 6.5 Kb, about 7.0 Kb, about 7.5 Kb, about 8.0 Kb, about 8.5 Kb, about 9.0 Kb, about 9.5 Kb, about 10 Kb, about 11 Kb, about 12 Kb, about 13 Kb, about 14 Kb, about 15 Kb, about 16 Kb, about 17 Kb, about 18 Kb, about 19 Kb, about 20 Kb in length, or greater).

### B. Upstream Regulatory Elements

Expression constructs disclosed herein can include one or more regulatory elements upstream of the transcriptional start site of a protein-encoding sequence (upstream elements), such as promoter sequences and/or hypersensitivity sequences.

In some instances described herein, the expression construct includes a hypersensitivity sequence derived from (e.g., containing a portion of, or variant thereof) a native intronic sequence found upstream from the transcriptional start site of CFTR (e.g., human CFTR). In some embodiments, the hypersensitivity sequence is derived from a native intronic sequence found, in its native position (e.g., according to GRch37/hg19), at least 5,000 nucleotides upstream from the transcriptional start site of CFTR (e.g., human CFTR) (e.g., between 5,000 and 50,000 nucleotides upstream from the transcriptional start site of CFTR (e.g., human CFTR)). In some embodiments, the hypersensitivity sequence is derived from a native intronic sequence found, in its native position (e.g., according to GRch37/hg19), at least 10,000 nucleotides upstream from the transcriptional start site of CFTR (e.g., human CFTR) (e.g., between 10,000 and 50,000 nucleotides upstream from the transcriptional start site of CFTR (e.g., human CFTR)). In some embodiments, the hypersensitivity sequence is derived from a native intronic sequence found, in its native position (e.g., according to GRch37/hg19), at least 20,000 nucleotides upstream from the transcriptional start site of CFTR (e.g., human CFTR) (e.g., between 20,000 and 50,000, between 30,000 and 50,000, or between 40,000 and 50,000 nucleotides upstream from the transcriptional start site of CFTR (e.g., human CFTR)).

For example, the present invention is based, in part, on discovery of a hypersensitivity sequence derived from a native human intronic sequence between 40,000 and 50,000 nucleotides upstream from the transcriptional start site human CFTR on chromosome 7, position 117,074,974-117,076,392 of Genome Reference Consortium Human Build 37 release 19 (GRch37/hg19; SEQ ID NO: 5). Such hypersensitivity sites (e.g., SEQ ID NO: 5) can enhance expression levels of genes operably linked thereto (e.g., operatively linked downstream). Thus, some embodiments of the invention feature a hypersensitivity sequence derived from (e.g., containing a portion of, or variant thereof) SEQ ID NO: 5. In some instances, a hypersensitivity sequence derived from SEQ ID NO: 5 has one or more (e.g., one, two, three, four, five, or more) sequences that are identical to at least 100 consecutive nucleotides within SEQ ID NO: 5, e.g., one or more (e.g., one, two, three, four, five, or more) sequences that are identical to at least 200, at least 300, at least 400, at least 500, at least 600, at least 700, at least 800, at least 900, at least 1000, at least 1100, at least 1200, at least 1300, or at least 1400 consecutive nucleotides within SEQ ID NO: 5. e.g., one or more (e.g., one, two, three, four, five, or more) sequences that are identical to 100-1500, 200-1200, 300-1000, or 400-800 consecutive nucleotides within SEQ ID NO: 5.

In some embodiments, any of the aforementioned hypersensitivity sequences derived from a native intronic sequence found upstream from the transcriptional start site of CFTR (e.g., human CFTR) has one or more (e.g., one, two, three, four, five, or more) sequences that are at least 90% identical (e.g., at least 95% identical, at least 96% identical, at least 97% identical, at least 98% identical, at least 99% identical, or 100% identical) to at least 100 consecutive nucleotides within SEQ ID NO: 5. For instance, any of the aforementioned hypersensitivity sequences derived from a native intronic sequence found upstream from the transcriptional start site of CFTR (e.g., human CFTR) has one or more (e.g., one, two, three, four, five, or more) sequences that are at least 90% identical (e.g., at least 95% identical, at least 96% identical, at least 97% identical, at least 98% identical, at least 99% identical, or 100% identical) to at least 200, at least 300, at least 400, at least 500, at least 600, at least 700, at least 800, at least 900, at least 1000, at least 1100, at least 1200, at least 1300, or at least 1400 consecutive nucleotides within SEQ ID NO: 5.

In some instances, a hypersensitivity sequence derived from a native intronic sequence found upstream from the transcriptional start site of CFTR (e.g., human CFTR) has one or more (one, two, three, four, five, or more) sequences that are identical to at least 100 consecutive nucleotides of SEQ ID NO: 5, e.g., at least 200, at least 300, at least 400, at least 500, at least 600, at least 700, at least 800, at least 900, at least 1000, at least 1100, at least 1200, at least 1300, or at least 1400 consecutive nucleotides of SEQ ID NO: 5).

In some instances, a hypersensitivity sequences derived from a native intronic sequence found upstream from the transcriptional start site of CFTR (e.g., human CFTR) has one or more (one, two, three, four, five, or more) sequences that are at least 90% identical (e.g., at least 95% identical, at least 96% identical, at least 97% identical, at least 98% identical, or at least 99% identical) to at least 100 consecutive nucleotides of SEQ ID NO: 5, e.g., at least 200, at least 300, at least 400, at least 500, at least 600, at least 700, at least 800, at least 900, at least 1000, at least 1100, at least 1200, at least 1300, or at least 1400 consecutive nucleotides of SEQ ID NO: 5).

In some embodiments, the expression construct includes a sequence having at least 75, 80, 85, 90, 95, 98, 99, 99.5 or 99.9% sequence identity or having 100% sequence identity to SEQ ID NO: 5.

Expression constructs described herein may include a promoter sequence. A promoter sequence may include a native sequence derived from the endogenous promoter of a respiratory gene, e.g., a CFTR gene. In some embodiments, the promoter sequence includes a native sequence of the same gene to which it is operably linked. For example, a CFTR coding sequence can be operably linked to, and be under the control of, a sequence derived from the native CFTR genetic locus, such as a sequence upstream of the CFTR transcription start site. In some embodiments, the promoter sequence and coding sequence are derived from native sequences of the same species. For example, an expression construct may include a CFTR native promoter sequence from the human genome and the CFTR coding sequence from the human genome, or a functional variant thereof.

In some embodiments, promoter sequences included in expression constructs disclosed herein are tissue-specific promoters in that, in normal operation, they drive expression only when present in certain tissue types, such as airway epithelium. In some embodiments, a promoter sequence used in an expression construct is not tissue-specific but is capable of driving expression in any tissue type. In some embodiments, the promoter is an inducible promoter. In some embodiments, the promoter sequence is a constitutive promoter. In some embodiments, the construct described herein comprises an elongation factor 1 alpha (EF1A) promoter sequence (e.g., SEQ ID NO: 8 or a functional variant thereof). In some instances, the construct described herein includes a cytomegalovirus (CMV) enhancer/beta-actin (CAG) promoter (e.g., SEQ ID NO: 9 or a functional variant thereof), an interphotoreceptor retinoid-binding protein (IBRP) promoter, a rhodopsin kinase (RK) promoter (e.g., G protein-coupled receptor kinase 1 (GRK1) promoter), an SV40 promoter, a dihydrofolate reductase promoter, a β-actin promoter, a phosphoglycerol kinase (PGK) promoter, or functional variants of any of the aforementioned promoters.

In some instances, a construct of the invention includes an inducible promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources. Many other systems have been described and can be readily selected by one of skill in the art. Examples of inducible promoters regulated by exogenously supplied promoters include zinc-inducible sheep metallothionine (MT) promoters, dexamethasone-inducible mouse mammary tumor virus promoters. T7 polymerase promoter systems, ecdysone insect promoters, tetracycline-repressible systems, tetracycline-inducible systems, RU486-inducible systems, and rapamycin-inducible systems. Still other types of inducible promoters which may be useful in this context are those which are regulated by a specific physiological state, e.g., temperature, acute phase, a particular differentiation state of the cell, or in replicating cells only.

In addition to the sequence sufficient to direct transcription, a promoter sequence of the invention can also include sequences of other regulatory elements that are involved in modulating transcription (e.g., hypersensitivity sequences, enhancers, Kozak sequences, and introns). In some embodiments, regulatory elements are derived from native sequences of the same species as the gene to which they are operably linked in expression constructs.

In some instances, a hypersensitivity sequence (e.g., any of the hypersensitivity sequences described herein) is operably linked (e.g., directly or indirectly) upstream of a promoter that is derived from a native intronic sequence found upstream from the transcriptional start site of CFTR (e.g., human CFTR), e.g., a native promoter. In some instances, the promoter is derived from a native sequence that extends, in its native position (e.g., according to GRch37/hg19), no more than 5,000 nucleotides upstream from the transcriptional start site of CFTR (e.g., human CFTR) (e.g., no more than 4,500, 4,000, 3,500, or 3,000 nucleotides upstream from the transcriptional start site of CFTR (e.g., human CFTR)).

In some instances, the promoter is derived from human chromosome 7, position 117,118,786-117,120,280 of GRch37/hg19 (e.g., a promoter comprising SEQ ID NO: 6, or a functional variant thereof (e.g., SEQ ID NO: 7)). Such promoters (e.g., SEQ ID NO: 6 or SEQ ID NO: 7) can enhance expression levels of genes operably linked thereto (e.g., operatively linked downstream). Thus, some embodiments of the invention feature a promoter derived from (e.g., containing a portion of, or variant thereof) SEQ ID NO: 6. In some instances, a promoter derived from SEQ ID NO: 6 has one or more (e.g., one, two, three, four, five, or more) sequences that are identical to at least 100 consecutive nucleotides within SEQ ID NO: 6. e.g., one or more (e.g., one, two, three, four, five, or more) sequences that are identical to at least 200, at least 300, at least 400, at least 500, at least 600, at least 700, at least 800, at least 900, at least 1000, at least 1100, at least 1200, at least 1300, or at least 1400 consecutive nucleotides within SEQ ID NO: 6, e.g., one or more (e.g., one, two, three, four, five, or more) sequences that are identical to 100-1500, 200-1200, 300-1000, or 400-800 consecutive nucleotides within SEQ ID NO: 6.

In some embodiments, any of the aforementioned promoters derived from a native intronic sequence found upstream from the transcriptional start site of CFTR (e.g., human CFTR) has one or more (e.g., one, two, three, four, five, or more) sequences that are at least 90% identical (e.g., at least 95% identical, at least 96% identical, at least 97% identical, at least 98% identical, at least 99% identical, or 100% identical) to at least 100 consecutive nucleotides within SEQ ID NO: 6. For instance, any of the aforementioned promoters derived from a native intronic sequence found upstream from the transcriptional start site of CFTR (e.g., human CFTR) may have one or more (e.g., one, two, three, four, five, or more) sequences that are at least 90% identical (e.g., at least 95% identical, at least 96% identical, at least 97% identical, at least 98% identical, at least 99% identical, or 100% identical) to at least 200, at least 300, at least 400, at least 500, at least 600, at least 700, at least 800, at least 900, at least 1000, at least 1100, at least 1200, at least 1300, or at least 1400 consecutive nucleotides within SEQ ID NO: 6.

In some instances, a promoter sequence derived from a native intronic sequence found upstream from the transcriptional start site of CFTR (e.g., human CFTR) has one or more (one, two, three, four, five, or more) sequences that are identical to at least 100 consecutive nucleotides of SEQ ID NO: 6, e.g., at least 200, at least 300, at least 400, at least 500, at least 600, at least 700, at least 800, at least 900, at least 1000, at least 1100, at least 1200, at least 1300, or at least 1400 consecutive nucleotides of SEQ ID NO: 6).

In some instances, a promoter sequence derived from a native intronic sequence found upstream from the transcriptional start site of CFTR (e.g., human CFTR) has one or more (one, two, three, four, five, or more) sequences that are at least 90% identical (e.g., at least 95% identical, at least 96% identical, at least 97% identical, at least 98% identical, or at least 99% identical) to at least 100 consecutive nucleotides of SEQ ID NO: 6 or SEQ ID NO: 7, e.g., at least 200, at least 300, at least 400, at least 500, at least 600, at least 700, at least 800, at least 900, at least 1000, at least 1100, at least 1200, at least 1300, or at least 1400 consecutive nucleotides of SEQ ID NO: 6 or SEQ ID NO: 7.

In some embodiments, the expression construct includes a sequence having at least 75, 80, 85, 90, 95, 98, 99, 99.5 or 99.9% sequence identity or having 100% sequence identity to SEQ ID NO: 6 or SEQ ID NO: 7.

In some embodiments, the expression construct includes any of the aforementioned hypersensitivity sequences derived from a native intronic sequence found upstream from the transcriptional start site of CFTR (e.g., human CFTR) (e.g., derived from SEQ ID NO: 5) operatively linked upstream to any of the aforementioned promoter sequences derived from a native intronic sequence found upstream from the transcriptional start site of CFTR (e.g., human CFTR), e.g., any of the aforementioned promoter sequences that extend, in its native position (e.g., according to GRch37/hg19), no more than 5,000 nucleotides upstream from the transcriptional start site of CFTR (e.g., human CFTR) (e.g., no more than 4,500, 4,000, 3,500, or 3,000 nucleotides upstream from the transcriptional start site of CFTR (e.g., human CFTR)), e.g., derived from SEQ ID NO: 6. Thus, in some instances, the expression construct of the invention includes a hypersensitivity sequence derived from SEQ ID NO: 5, which is operatively linked upstream to a promoter sequence derived from SEQ ID NO: 6.

In some instances, the hypersensitivity sequence (e.g., the hypersensitivity sequence derived from SEQ ID NO: 5) is operatively linked to the promoter (e.g., the promoter derived from SEQ ID NO: 6, e.g., SEQ ID NO: 7) by an intervening sequence (e.g., an inert intervening sequence) of no more than 500 nucleotides (e.g., no more than 400, 300, 200, or 100 nucleotides), e.g., from 0-10, from 2-20, from 2-10, or from 4-8 nucleotides. In some instances, the hypersensitivity sequence (e.g., the hypersensitivity sequence derived from SEQ ID NO: 5) is operatively linked to the promoter (e.g., the promoter derived from SEQ ID NO: 6, e.g., SEQ ID NO: 7) by an intervening sequence that consists of, or comprises, a restriction cut site, e.g., a sticky end or overhang of the type IIs restriction enzyme cut site (e.g., TTTT or AAAA).

### C. Scaffold/Matrix Attachment Regions

In some embodiments, an expression construct disclosed herein includes a scaffold-matrix attachment regions (S/MAR). Without being bound by theory, it is believed that S/MAR elements can help establish long-term gene expression from a DNA vector through the interaction of the S/MAR element with the nuclear matrix. Known S/MAR constructs include the human IFN-γ S/MAR (SEQ ID NO: 2) and the human APOB S/MAR (NCBI Gene ID 106632268). Other known S/MAR elements can be included in expression constructs disclosed herein, as can functional variants thereof. In some embodiments, a variant (SEQ ID NO: 10) of the IFN-γ S/MAR comprising tandem repeats of a functional portion of the IFN-γ S/MAR is included in expression constructs provided herein. In some embodiments, the expression construct includes a sequence having at least 75, 80, 85, 90, 95, 98, 99, 99.5 or 99.9% sequence identity or having 100% sequence identity to SEQ ID NO: 2 or 10. S/MAR sequences can be operably linked either 5′ or 3′ to a coding sequence of an expression construct. In some embodiments, an S/MAR sequence is linked to the 3′ end of a coding sequence (e.g., a CFTR-encoding sequence).

### D. Arrangement Expression Construct Elements

Upstream elements, promoter sequences, coding sequences, S/MAR sequences, and other elements (e.g., polyadenylation sequences) can be included in expression constructs described herein in any suitable order that provides for effective expression and/or persistence.

In some embodiments, an expression construct includes, operably linked (e.g., directly or indirectly) in a 5′ to 3′ direction, a promoter (e.g., a constitutive promoter, such as an EF1A promoter), a coding sequence (e.g., a therapeutic protein-encoding sequence, e.g., a CFTR-encoding sequence, e.g., SEQ ID NO: 1, or a functional variant thereof), and an S/MAR sequence (e.g., an IFN-γ S/MAR sequence (e.g., SEQ ID NO: 2), or a functional variant thereof (e.g., SEQ ID NO: 10)). In some embodiments, the expression construct further includes an inert enhancer (e.g., SEQ ID NO: 3, or a functional variant thereof) or an inert insulator (e.g., SEQ ID NO: 4, or a functional variant thereof) operably linked 5′ to the promoter.

In some embodiments, an expression construct includes, operably linked (e.g., directly or indirectly) in a 5′ to 3′ direction, a hypersensitivity sequence (e.g., a hypersensitivity sequence derived from SEQ ID NO: 5), a promoter sequence (e.g., a promoter sequence derived from SEQ ID NO: 6), and a coding sequence (e.g., a therapeutic protein-encoding sequence, e.g., a CFTR-encoding sequence, e.g., SEQ ID NO: 1, or a functional variant thereof).

### III. Nucleic Acid Vectors

Provided herein are nucleic acid vectors (e.g., DNA vectors, e.g., nonviral DNA vectors (e.g., naked DNA vectors), circular DNA vectors (e.g., supercoiled circular DNA vectors), and/or synthetic DNA vectors (e.g., synthetic circular DNA vectors) that include any of the expression constructs described herein, or components (e.g., regulatory elements) or combinations thereof. In some instances, a nucleic acid vector is a circular DNA vector (e.g., a synthetic circular DNA vector) comprising a promoter, a transgene, and a scaffold matrix attachment region (S/MAR) sequence, wherein the transgene is a therapeutic protein-encoding sequence derived from a protein expressed in respiratory epithelium (e.g., CFTR, or a functional variant thereof), and wherein the circular DNA vector lacks a drug resistance gene. In some instances, the circular DNA vector includes an EF1A promoter sequence.

In some instances, a nucleic acid vector (e.g., a circular DNA vector, e.g., a synthetic circular DNA vector) includes a hypersensitivity sequence (e.g., a hypersensitivity sequence comprising SEQ ID NO: 5, or a functional variant thereof) operably linked 5′ to a transgene (e.g., a transgene that is a therapeutic protein-encoding sequence derived from a protein expressed in respiratory epithelium (e.g., CFTR, or a functional variant thereof)). In some instances, the hypersensitivity sequence is operably linked upstream from a promoter sequence (e.g., a promoter sequence derived from a native CFTR promoter, e.g., a promoter sequence derived from SEQ ID NO: 6, e.g., SEQ ID NO: 7).

Nucleic acid vectors containing regulatory elements described herein can be produced according to known methods as plasmid DNA vectors, nanoplasmid vectors (as described in, e.g., International Patent Publication Nos. WO 2008/153733 and WO 2014/035457), minicircle ONA vectors (as described in, e.g., U.S. Pat. Nos. 8,828,726 and 9,233,174), mini-intronic plasmids (described in, e.g., Lu et al., Mol. Ther. 2013, 21:954 and U.S. Pat. No. 9,347,073), synthetic circular DNA vectors as described herein and in International Patent Publication No. WO 2019/178500, closed-ended ONA vectors (as described, e.g., in U.S. Patent Publication Nos. 2020/0283794 and 2021/0071197), doggybone DNA vectors (as described, e.g., in U.S. Patent Publication No. 2015/0329902 and U.S. Pat. No. 9,499,847), or ministring ONA vectors (as described, e.g., in U.S. Pat. No. 9,290,778 and USRE48908E1). In particular embodiments, any of the nucleic acid vectors described herein (e.g., nonviral DNA vectors (e.g., naked DNA vectors), circular DNA vectors (e.g., supercoiled circular ONA vectors), and/or synthetic DNA vectors (e.g., synthetic circular DNA vectors)) include a therapeutic sequence (e.g., a respiratory gene-encoding sequence, such as CFTR). In some instances, a nucleic acid vector of the invention is a nonviral DNA vector (e.g., a naked DNA vector), a circular DNA vector (e.g., a supercoiled circular DNA vector), and/or a synthetic ONA vector (e.g., a synthetic circular DNA vector)), which comprises: a promoter, a transgene, and a scaffold matrix attachment region (S/MAR) sequence, wherein the transgene is a therapeutic protein-encoding sequence derived from a protein expressed in respiratory epithelium, and wherein the circular DNA vector lacks a drug resistance gene. In some instances, a nucleic acid vector of the invention is a nonviral DNA vector (e.g., a naked DNA vector), a circular DNA vector (e.g., a supercoiled circular DNA vector), and/or a synthetic DNA vector (e.g., a synthetic circular DNA vector)), which comprises: a hypersensitivity sequence operably linked 5′ to a transgene, wherein the hypersensitivity sequence comprises SEQ ID NO: 5, or a functional variant thereof.

In some instances, nucleic acid vectors of the invention are synthetic circular DNA vectors that persist intracellularly (e.g., in dividing or in quiescent cells, such as post-mitotic cells) as episomes, e.g., in a manner similar to AAV vectors. In any of the embodiments described herein, a synthetic circular DNA vector may be a non-integrating vector. Synthetic circular DNA vectors provided herein can be naked DNA vectors, devoid of components inherent to viral vectors (e.g., viral proteins) and bacterial plasmid DNA, such as immunogenic components (e.g., immunogenic bacterial signatures (e.g., CpG islands or CpG motifs)) or components additionally, or otherwise associated with reduced persistence (e.g., CpG islands or CpG motifs). Synthetic circular DNA vectors produced as described herein feature one or more therapeutic sequences and may lack plasmid backbone elements (e.g., bacterial elements such as (i) a bacterial origin of replication and/or (ii) a drug resistance gene) and a recombination site. Such synthetic circular DNA vectors can be synthesized through various means known in the art and described herein (see, e.g., Examples 1 and 2). Synthesis methods may involve use of phage polymerase, such as Phi29 polymerase, as a replication tool using, e.g., rolling circle amplification. Particular methods of cell-free synthesis of synthetic circular DNA vectors are further described in International Patent Publication No. WO 2019/178500, which is incorporated herein by reference.

In other embodiments, therapeutic circular DNA vectors described herein can be non-synthetic vectors (e.g., containing bacterial backbone sequences and/or recombination sites), such as plasmid DNA vectors, minicircle DNA vectors, mini-intronic plasmid vectors.

In some instances, the nucleic acid vector is an in vivo-produced circular DNA vector that lacks a recombination site and a selectable marker (e.g., drug resistance gene), e.g., by using engineered bacterial cells to produce circular DNA vectors from a parental plasmid. Bacterial cells (e.g., E. coli) can be engineered to contain a Rep gene encoding a bacterial replication protein integrated into the bacterial genome. The engineered cells can be transfected with a parental plasmid having a vector sequence and a backbone sequence. The vector sequence includes an ori sequence corresponding to the Rep gene and does not include a selectable marker. The backbone sequence includes a selectable marker and does not include the ori sequence included in the vector sequence. The parental plasmid also has restriction enzyme recognition sequences or site-specific recombination sequences flanking the vector sequence arranged so that the plasmid backbone sequence can be separated from the vector sequence inside the cell by restriction digestion or site-specific recombination. In the case of restriction digestion, the circular DNA vector is then formed by self-ligation of the vector sequence. In the case of site-specific recombination, the circular DNA vector is formed as recombination is completed. Expression of the rep protein after separation of the vector sequence and formation of the circular DNA vector can maintain the circular DNA vector at a high copy number, despite the circular ONA vector lacking a selectable marker. In contrast, maintenance of the plasmid backbone sequence in the engineered bacterial cell after separation can be avoided by changing the culture conditions to remove selective pressure for the selectable marker. Culturing of a population of bacterial cells with a high copy number of circular DNA vector under conditions in which the parental plasmid is not maintained can efficiently produce a high yield of highly pure circular DNA vector. Such methods for in vivo production of circular DNA vectors that lack a recombination site and a selectable marker are described in U.S. Provisional Patent Application No. 63/291,871.

In some instances, synthetic circular DNA vectors provided herein are naked DNA vectors and are devoid of components inherent to viral vectors (e.g., viral proteins) and bacterial plasmid DNA, such as immunogenic components (e.g., immunogenic bacterial signatures (e.g., CpG motifs) or components additionally or otherwise associated with reduced persistence (e.g., CpG islands). For example, in some embodiments, the synthetic circular DNA vector contains DNA in which at least 50% (e.g., at least 60%, at least 70%, at least 80%, at least 90%, at least 95%, at least 97%, at least 99%, or essentially all) of the DNA lacks one or more elements of bacterial plasmid DNA, such as immunogenic components (e.g., immunogenic bacterial signatures (e.g., CpG motifs) or components additionally or otherwise associated with reduced persistence (e.g., CpG islands). In some embodiments, at least 50% (e.g., at least 60%, at least 70%, at least 80%, at least 90%, at least 95%, at least 97%, at least 99%, or essentially all) of the DNA lacks CpG methylation. In some embodiments, the synthetic circular DNA vector contains DNA in which at least 50% (e.g., at least 60%, at least 70%, at least 80%, at least 90%, at least 95%, at least 97%, at least 99%, or essentially all) of the DNA lacks bacterial methylation signatures, such as Dam methylation and Dem methylation. For examples, in some embodiments, the synthetic circular DNA vector contains DNA in which at least 50% (e.g., at least 60%, at least 70%, at least 80%, at least 90%, at least 95%, at least 97%, at least 99%, or essentially all) of the GATC sequences are unmethylated (e.g., by Dam methylase). Additionally, or alternatively, the synthetic circular DNA vector contains DNA in which at least 50% (e.g., at least 60%, at least 70%, at least 80%, at least 90%, at least 95%, at least 97%, at least 99%, or essentially all) of the CCAGG sequences and/or CCTGG sequences are unmethylated (e.g., by Dom methylase).

In some embodiments, the synthetic circular DNA vector is persistent in vivo (e.g., the therapeutic circular DNA vector exhibits improved expression persistence (e.g., intra-cellular persistence and/or trans-generational persistence) and/or therapeutic persistence relative to a reference vector, e.g., a circular DNA vector produced in bacteria or having one or more bacterial signatures not present in the vector of the invention, e.g., plasmid DNA). In some embodiments, expression persistence of the synthetic circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, intra-cellular persistence of the synthetic circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, trans-generational persistence of the therapeutic circular DNA vector (e.g., synthetic circular DNA vector) is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, therapeutic persistence of the synthetic circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven fold, eight-fold, nine-fold, ten-fold, or more) greater than a reference vector. In some embodiments, the reference vector is a circular vector or plasmid that (a) has the same therapeutic sequence as a synthetic circular DNA vector to which it is being compared, and (b) is produced in bacteria and/or has one or more bacterial signatures not present in the synthetic circular DNA vector to which it is being compared, which signatures may include, for example, an antibiotic resistance gene or a bacterial origin of replication.

In some embodiments, expression of a synthetic circular DNA vector persists for one week, two weeks, three weeks, four weeks, six weeks, two months, three months, four months, five months, six months, seven months, eight months, nine months, ten months, eleven months, one year, or longer after administration. In particular embodiments, the synthetic circular DNA vector exhibits intra-cellular persistence and/or trans-generational persistence of one week, two weeks, three weeks, four weeks, six weeks, two months, three months, four months, five months, six months, seven months, eight months, nine months, ten months, eleven months, one year, or longer after administration. In some embodiments, therapeutic persistence of a synthetic circular DNA vector endures for one week, two weeks, three weeks, four weeks, six weeks, two months, three months, four months, five months, six months, seven months, eight months, nine months, ten months, eleven months, one year, or longer after administration.

In some embodiments, expression and/or therapeutic effect of the synthetic circular DNA vector persists for one week to four weeks, from one month to four months, or from four months to one year (e.g., at least one week, at least two weeks, at least one month, or longer). In some embodiments, the expression level of the therapeutic circular DNA vector (e.g., synthetic circular ONA vector) does not decrease by more than 90%, by more than 50%, or by more than 10% in the 1 week or more, e.g., 2 weeks, 3 weeks, 5 weeks, 7 weeks, 9 weeks or more, 13 weeks or more, 18 weeks or more following transfection from levels observed within the first 1, 2, or 3 days.

The synthetic circular DNA vector may be monomeric, dimeric, trimeric, tetrameric, pentameric, hexameric, etc. In some preferred embodiments, the synthetic circular DNA vector is monomeric. In some embodiments, the synthetic circular DNA vector is supercoiled, e.g., following treatment with a topoisomerase (e.g., gyrase). In some embodiments, the synthetic circular DNA vector is a monomeric, supercoiled circular DNA molecule. In some embodiments, the synthetic circular DNA vector is nicked. In some embodiments, the synthetic circular DNA vector is open circular (relaxed open circular). In some embodiments, the synthetic circular DNA vector is double-stranded circular.

Therapeutic circular DNA vectors (e.g., synthetic therapeutic circular DNA vectors) described herein contain a therapeutic sequence, which may include one or more protein-coding domains and/or one or more non-protein coding domains. Therapeutic sequences can include any of the expression constructs disclosed herein.

In some embodiments, the therapeutic sequence encodes a therapeutic protein (e.g., a therapeutic replacement protein) which is a monomeric protein (e.g., a monomeric protein with secondary structure under physiological conditions, e.g., a monomeric protein with secondary and tertiary structure under physiological conditions, e.g., a monomeric protein with secondary, tertiary, and quaternary structure under physiological conditions). Additionally, or alternatively, the therapeutic sequence may encode a multimeric therapeutic protein (e.g., a dimeric therapeutic protein (e.g., a homodimeric therapeutic protein or heterodimeric therapeutic protein), a trimeric therapeutic protein, etc.)

In some embodiments involving synthetic circular DNA vectors, the therapeutic sequence is from 0.1 Kb to 100 Kb in length (e.g., the therapeutic gene sequence is from 0.2 Kb to 90 Kb, from 0.5 Kb to 80 Kb, from 1.0 Kb to 70 Kb, from 1.5 Kb to 60 Kb, from 2.0 Kb to 50 Kb, from 2.5 Kb to 45 Kb, from 3.0 Kb to 40 Kb, from 3.5 Kb to 35 Kb, from 4.0 Kb to 30 Kb, from 4.5 Kb to 25 Kb, from 4.6 Kb to 24 Kb, from 4.7 Kb to 23 Kb, from 4.8 Kb to 22 Kb, from 4.9 Kb to 21 Kb, from 5.0 Kb to 20 Kb, from 5.5 Kb to 18 Kb, from 6.0 Kb to 17 Kb, from 6.5 Kb to 16 Kb, from 7.0 Kb to 15 Kb, from 7.5 Kb to 14 Kb, from 8.0 Kb to 13 Kb, from 8.5 Kb to 12.5 Kb, from 9.0 Kb to 12.0 Kb, from 9.5 Kb to 11.5 Kb, or from 10.0 Kb to 11.0 Kb in length, e.g., from 0.1 Kb to 0.5 Kb, from 0.5 Kb to 1.0 Kb, from 1.0 Kb to 2.5 Kb, from 2.5 Kb to 4.5 Kb, from 4.5 Kb to 8 Kb, from 8 Kb to 10 Kb, from 10 Kb to 15 Kb, from 15 Kb to 20 Kb in length, or greater, e.g., from 0.1 Kb to 0.25 Kb, from 0.25 Kb to 0.5 Kb, from 0.5 Kb to 1.0 Kb, from 1.0 Kb to 1.5 Kb, from 1.5 Kb to 2.0 Kb, from 2.0 Kb to 2.5 Kb, from 2.5 Kb to 3.0 Kb, from 3.0 Kb to 3.5 Kb, from 3.5 Kb to 4.0 Kb, from 4.0 Kb to 4.5 Kb, from 4.5 Kb to 5.0 Kb, from 5.0 Kb to 5.5 Kb, from 5.5 Kb to 6.0 Kb, from 6.0 Kb to 6.5 Kb, from 6.5 Kb to 7.0 Kb, from 7.0 Kb to 7.5 Kb, from 7.5 Kb to 8.0 Kb, from 8.0 Kb to 8.5 Kb, from 8.5 Kb to 9.0 Kb, from 9.0 Kb to 9.5 Kb, from 9.5 Kb to 10 Kb, from 10 Kb to 10.5 Kb, from 10.5 Kb to 11 Kb, from 11 Kb to 11.5 Kb, from 11.5 Kb to 12 Kb, from 12 Kb to 12.5 Kb, from 12.5 Kb to 13 Kb, from 13 Kb to 13.5 Kb, from 13.5 Kb to 14 Kb, from 14 Kb to 14.5 Kb, from 14.5 Kb to 15 Kb, from 15 Kb to 15.5 Kb, from 15.5 Kb to 16 Kb, from 16 Kb to 16.5 Kb, from 16.5 Kb to 17 Kb, from 17 Kb to 17.5 Kb, from 17.5 Kb to 18 Kb, from 18 Kb to 18.5 Kb, from 18.5 Kb to 19 Kb, from 19 Kb to 19.5 Kb, from 19.5 Kb to 20 Kb, from 20 Kb to 21 Kb, from 21 Kb to 22 Kb, from 22 Kb to 23 Kb, from 23 Kb to 24 Kb, from 24 Kb to 25 Kb in length, or greater, e.g., about 4.5 Kb, about 5.0 Kb, about 5.5 Kb, about 6.0 Kb, about 6.5 Kb, about 7.0 Kb, about 7.5 Kb, about 8.0 Kb, about 8.5 Kb, about 9.0 Kb, about 9.5 Kb, about 10 Kb, about 11 Kb, about 12 Kb, about 13 Kb, about 14 Kb, about 15 Kb, about 16 Kb, about 17 Kb, about 18 Kb, about 19 Kb, about 20 Kb in length, or greater). In some embodiments, the therapeutic sequence is at least 10 Kb (e.g., from 10 Kb to 15 Kb, from 15 Kb to 20 Kb, or from 20 Kb to 30 Kb; e.g., from 10 Kb to 13 Kb, from 10 Kb to 12 Kb, or from 10 Kb to 11 Kb; e.g., from 10-11 Kb, from 11-12 Kb, from 12-13 Kb, from 13-14 Kb, or from 14-15 Kb). In some embodiments, the therapeutic sequence is at least 1,100 bp in length (e.g., from 1,100 bp to 10,000 bp, from 1,100 bp to 8,000 bp, or from 1,100 bp to 5,000 bp in length). In some embodiments, the therapeutic sequence is at least 2,500 bp in length (e.g., from 2,500 bp to 15,000 bp, from 2,500 bp to 10,000 bp, or from 2,500 bp to 5,000 bp in length; e.g., from 2,500 bp to 5,000 bp, from 5,000 bp to 7,500 bp, from 7,500 bp to 10,000 bp, from 10,000 bp to 12,500 bp, or from 12,500 bp to 15,000 bp). In some embodiments, the therapeutic sequence is at least 8,000 bp, at least 9,000 bp, at least 10,000 bp, at least 11,000 bp, at least 12,000 bp at least 13,000 bp, at least 14,000 bp, at least 15,000 bp, at least 16,000 bp (e.g., 11,000 bp to 16,000 bp, 12,000 bp to 16,000 bp, 13,000 bp to 16,000 bp, 14,000 bp to 16,000 bp, or 15,000 bp to 16,000 bp). In particular embodiments, the therapeutic sequence is sufficiently large to encode a protein and is not an oligonucleotide therapy (e.g., not an antisense, siRNA, shRNA therapy, etc.).

In some embodiments involving circular DNA vectors (e.g., synthetic circular DNA vectors), the 3′ end of the therapeutic sequence is connected to the 5′ end of the therapeutic sequence in a therapeutic circular DNA vector (e.g., synthetic circular DNA vector) by a non-bacterial sequence of no more than 30 bp (e.g., from 3 bp to 24 bp, from 4 bp to 18 bp, from 5 bp to 12 bp, or from 6 bp to 10 bp; e.g., from 3 bp to 5 bp, from 4 bp to 6 bp, from 8 bp to 12 bp, from 12 bp to 18 bp, from 18 bp to 24 bp, or from 24 bp to 30 bp; e.g., 3 bp, 4 bp, 5 bp, 6 bp, 7 bp, or 8 bp). For example, the 3′ end of the therapeutic sequence may be connected to the 5′ end of the therapeutic sequence by a non-bacterial sequence corresponding to sticky end or overhang of the type IIs restriction enzyme cut site (e.g., TTTT or AAAA).

Generally, a synthetic circular DNA vector is capable of having a higher ratio of therapeutic sequence to non-therapeutic sequence (e.g., sequence connecting the 3′ end of the therapeutic sequence to the 5′ end of the therapeutic sequence), relative to a non-synthetic circular DNA vector (e.g. a circular DNA vector made in vivo, such as a plasmid, which contains a bacterial backbone limiting the ratio). In some instances, the ratio of therapeutic sequence (e.g., from the 5′ end of any functional upstream elements to the 3′ end of any downstream functional elements, such as an S/MAR or polyA tail) to non-therapeutic sequence (e.g., a restriction site overhang, an origin of replication, and/or an inert artifact of cloning) in a circular DNA vector (e.g., synthetic circular DNA vector) of the invention is at least 10 (e.g., a therapeutic sequence is about 5000 bp and the non-therapeutic sequence is less than about 500 bp). For example, in some instances, the ratio of therapeutic sequence (e.g., from the 5′ end of any functional upstream elements to the 3′ end of any downstream functional elements, such as an S/MAR or polyA tail) to non-therapeutic sequence (e.g., a restriction site overhang, an origin of replication, and/or an inert artifact of cloning) in a circular DNA vector (e.g., synthetic circular DNA vector) of the invention is at least 50 (e.g., a therapeutic sequence is about 5000 bp and the non-therapeutic sequence is less than about 100 bp). In some instances, the ratio of therapeutic sequence (e.g., from the 5′ end of any functional upstream elements to the 3′ end of any downstream functional elements, such as an S/MAR or polyA tail) to non-therapeutic sequence (e.g., a restriction site overhang, an origin of replication, and/or an inert artifact of cloning) in a circular DNA vector (e.g., synthetic circular DNA vector) of the invention is at least 100 (e.g., a therapeutic sequence is about 8000 bp and the non-therapeutic sequence is less than about 80 bp). In some instances, the ratio of therapeutic sequence (e.g., from the 5′ end of any functional upstream elements to the 3′ end of any downstream functional elements, such as an S/MAR or polyA tail) to non-therapeutic sequence (e.g., a restriction site overhang, an origin of replication, and/or an inert artifact of cloning) in a circular DNA vector (e.g., synthetic circular DNA vector) of the invention is at least 500 (e.g., a therapeutic sequence is about 8000 bp and the non-therapeutic sequence is less than about 16 bp). In some instances, the ratio of therapeutic sequence (e.g., from the 5′ end of any functional upstream elements to the 3′ end of any downstream functional elements, such as an S/MAR or polyA tail) to non-therapeutic sequence (e.g., a restriction site overhang, an origin of replication, and/or an inert artifact of cloning) in a circular DNA vector (e.g., synthetic circular DNA vector) of the invention is at least 1,000 (e.g., a therapeutic sequence is about 8000 bp and the non-therapeutic sequence is less than about 8 bp). In some instances, the ratio of therapeutic sequence (e.g., from the 5′ end of any functional upstream elements to the 3′ end of any downstream functional elements, such as an S/MAR or polyA tail) to non-therapeutic sequence (e.g., a restriction site overhang, an origin of replication, and/or an inert artifact of cloning) in a circular DNA vector (e.g., synthetic circular DNA vector) of the invention is about 2,000 (e.g., a therapeutic sequence is about 8000 bp and the non-therapeutic sequence is about 4 bp, e.g., corresponding to a restriction site overhang, e.g., a type IIs restriction site overhang).

In some embodiments, the therapeutic sequence includes a reporter sequence in addition to a therapeutic protein-encoding domain or a therapeutic non-protein encoding domain. Such reporter genes can be useful in verifying therapeutic gene sequence expression, for example, in specific cells and tissues. Reporter sequences that may be provided in a transgene include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer. In some embodiments, the therapeutic sequence lacks a reporter sequence.

In some instances, any of the nucleic acid vectors of the invention (e.g., nonviral DNA vectors (e.g., naked DNA vectors), circular DNA vectors (e.g., supercoiled circular DNA vectors), and/or synthetic DNA vectors (e.g., synthetic circular DNA vectors)) encodes a self-replicating RNA molecule. Such self-replicating RNA molecules include replicase sequences derived from alphavirus, which are characterized as having positive-stranded replicons that are translated after delivery to a target cell into a replicase (or replicase-transcriptase). The replicase is translated as a polyprotein which auto-cleaves to provide a replication complex which creates genomic negative-strand copies of the positive-strand delivered RNA. These negative-strand transcripts can themselves be transcribed to give further copies of the positive-stranded parent RNA and also to give a subgenomic transcript (e.g., a modulatory sequence). Translation of the subgenomic transcript thus leads to in situ expression of the modulatory protein by the infected cell.

Non-limiting examples of alphaviruses from which replicase-encoding sequences of the present invention can be derived include Venezuelan equine encephalitis virus (VEE), Semliki Forest virus (SF), Sindbis virus (SIN). Eastern Equine Encephalitis virus (EEE), Western equine encephalitis virus (WEE), Everglades virus (EVE), Mucambo virus (MUC), Pixuna virus (PIX), Semliki Forest virus (SF), Middelburg virus (MID), Chikungunya virus (CHIK), O'Nyong-Nyong virus (ONN), Ross River virus (RR), Barmah Forest virus (BF), Getah virus (GET), Sagiyama virus (SAG), Bebaru virus (BEB), Mayaro virus (MAY), Una virus (UNA), Aura virus (AURA), Babanki virus (BAB), Highlands J virus (HJ), and Fort Morgan virus (FM). In particular instances of the invention, the self-replicating RNA molecule comprises a VEE replicase or a variant thereof.

Mutant or wild-type virus sequences can be used. For example, in some instances, the self-replicating RNA includes an attenuated TC83 mutant of VEE replicase. Other mutations in the replicase are contemplated herein, including replicase mutated replicases (e.g., mutated VEE replicases) obtained by in vitro evolution methods, e.g., as taught by Yingzhong et al., Sci Rep. 2019, 9:6932, the methodology of which is incorporated herein by reference.

In some instances, a self-replicating RNA molecule includes (I) a replicase-encoding sequence (e.g., an RNA sequence that encodes an RNA-dependent RNA polymerase which can transcribe RNA from the self-replicating RNA molecule) and (ii) a therapeutic protein-encoding sequence. The polymerase can be an alphavirus replicase, e.g., an alphavirus replicase comprising one, two, three, or all four alphavirus nonstructural proteins nsP1, nsP2, nsP3, and nsP4. In some instances, the polymerase is a VEE replicase, e.g., a VEE replicase comprising one, two, three, or all four alphavirus nonstructural proteins nsP1, nsP2, nsP3, and nsP4.

In other embodiments, the nucleic vector (e.g., a nonviral DNA vector (e.g., a naked DNA vector), a circular DNA vector (e.g., a supercoiled circular DNA vector), and/or a synthetic DNA vector (e.g., a synthetic circular DNA vector)) is a therapeutic nucleic vector that does not encode a reporter molecule, such as green fluorescent protein (GFP).

Expression constructs described herein can be assembled into viral vectors, such as vectors consisting of, or derived from, adeno-associated virus (AAV), adenovirus, Retroviridae family virus, parvovirus, coronavirus, rhabdovirus, paramyxovirus, picornavirus, alphavirus, herpes virus, or poxvirus.

In some instances, the nucleic acid vector is a non-viral DNA vector (e.g., the DNA vector is not encapsulated within a viral capsid). Additionally, or alternatively, in some embodiments, the nucleic acid vector is not encapsulated in an envelope (e.g., a lipid envelope) or a matrix (e.g., a polymer matrix) and is not physically associated with (e.g., covalently or non-covalently bound to) a solid structure (e.g., a particulate structure) prior to and upon administration to the individual. In some embodiments, the nucleic acid vector is untethered to any adjacent nucleic acid vectors such that, in a solution of nucleic acid vectors, each nucleic acid vector is free to diffuse independently of adjacent nucleic acid vectors. In some embodiments, the nucleic acid vector is associated with another agent in liquid solution, such as a charge-altering molecule or a stabilizing molecule.

The nucleic acid vector may be a naked DNA vector. i.e., not complexed with another agent (e.g., encapsulated within, conjugated to, or non-covalently bound to another agent). Naked DNA vectors may be co-formulated (e.g., in solution) with agents that are not complexed with the naked DNA vector, such as buffering agents and/or agents that are generally recognized as safe (GRAS) by the U.S. Food and Drug Administration.

### IV. Pharmaceutical Compositions

Nucleic acid vectors, such as DNA vectors (e.g., nonviral DNA vectors (e.g., naked DNA vectors), circular DNA vectors (e.g., supercoiled circular ONA vectors), and/or synthetic DNA vectors (e.g., synthetic circular DNA vectors)) described herein can be included in pharmaceutical compositions, e.g., formulated for administration to a subject, e.g., for treatment of a disease or disorder.

In some instances, a pharmaceutical composition includes a circular DNA vector (e.g., a synthetic circular DNA vector) comprising a promoter, a transgene, and a scaffold matrix attachment region (S/MAR) sequence, wherein the transgene is a therapeutic protein-encoding sequence derived from a protein expressed in respiratory epithelium (e.g., CFTR, or a functional variant thereof), and wherein the circular DNA vector lacks a drug resistance gene. In some instances, the circular DNA vector includes an EF1A promoter sequence.

In some instances, a pharmaceutical composition includes a nucleic acid vector (e.g., a circular DNA vector, e.g., a synthetic circular DNA vector) comprising a hypersensitivity sequence (e.g., a hypersensitivity sequence comprising SEQ ID NO: 5, or a functional variant thereof) operably linked 5′ to a transgene (e.g., a transgene that is a therapeutic protein-encoding sequence derived from a protein expressed in respiratory epithelium (e.g., CFTR, or a functional variant thereof)). In some instances, the hypersensitivity sequence is operably linked upstream from a promoter sequence (e.g., a promoter sequence derived from a native CFTR promoter, e.g., a promoter sequence derived from SEQ ID NO: 6, e.g., SEQ ID NO: 7).

In some embodiments, a pharmaceutical composition contains at least 1.0 mg nucleic acid vector (e.g., a nonviral DNA vector (e.g., a naked DNA vector), a circular DNA vector (e.g., a supercoiled circular DNA vector), and/or a synthetic DNA vector (e.g., a synthetic circular DNA vector) in a pharmaceutically acceptable carrier (e.g., from 1.0 mg to 10 g, from 1.0 mg to 5.0 g, from 1.0 mg to 1.0 g, from 1.0 mg to 500 mg, from 1.0 mg to 200 mg, from 1.0 mg to 100 mg, from 1.0 mg to 50 mg, from 1.0 mg to 25 mg, from 1.0 mg to 20 mg, from 1.0 mg to 15 mg, from 1.0 mg to 10 mg, from 1.0 mg to 5.0 mg, from 2.0 mg to 10 g, from 2.0 mg to 5.0 g, from 2.0 mg to 1.0 g, from 2.0 mg to 500 mg, from 2.0 mg to 200 mg, from 2.0 mg to 100 mg, from 2.0 mg to 50 mg, from 2.0 mg to 25 mg, from 2.0 mg to 20 mg, from 2.0 mg to 15 mg, from 2.0 mg to 10 mg, from 2.0 mg to 5.0 mg, from 5.0 mg to 10 g, from 5.0 mg to 5.0 g, from 5.0 mg to 1.0 g, from 5.0 mg to 500 mg, from 5.0 mg to 200 mg, from 5.0 mg to 100 mg, from 5.0 mg to 50 mg, from 5.0 mg to 25 mg, from 5.0 mg to 20 mg, from 5.0 mg to 15 mg, from 5.0 mg to 10 mg, from 10 mg to 10 g, from 10 mg to 5.0 g, from 10 mg to 1.0 g, from 10 mg to 500 mg, from 10 mg to 200 mg, from 10 mg to 100 mg, from 10 mg to 50 mg, from 10 mg to 25 mg, from 10 mg to 20 mg, or from 10 mg to 15 mg).

In some embodiments, a pharmaceutical composition contains at least 2.0 mg nucleic acid vector (e.g., a nonviral DNA vector (e.g., a naked DNA vector), a circular DNA vector (e.g., a supercoiled circular DNA vector), and/or a synthetic DNA vector (e.g., a synthetic circular DNA vector) in a pharmaceutically acceptable carrier. In some embodiments, a pharmaceutical composition produced by any of the synthetic methods described herein contains at least 5.0 mg synthetic circular DNA vector in a pharmaceutically acceptable carrier. In some embodiments, a pharmaceutical composition produced by any of the synthetic methods described herein contains at least 10.0 mg synthetic circular ONA vector in a pharmaceutically acceptable carrier.

In some embodiments, a pharmaceutical composition contains therapeutic circular DNA vector (e.g., synthetic circular DNA vector) that is at least 60% supercoiled monomer, at least 70% supercoiled monomer, at least 80% supercoiled monomer, or at least 90% supercoiled monomer (e.g., 60% to 80% supercoiled monomer, 60% to 90% supercoiled monomer, 60% to 95% supercoiled monomer, 60% to 99% supercoiled monomer, 60% to 99.5% supercoiled monomer, 60% to 99.9% supercoiled monomer, 65% to 80% supercoiled monomer, 65% to 90% supercoiled monomer, 65% to 95% supercoiled monomer, 65% to 99% supercoiled monomer, 65% to 99.5% supercoiled monomer, 65% to 99.9% supercoiled monomer, 70% to 80% supercoiled monomer, 70% to 90% supercoiled monomer, 70% to 95% supercoiled monomer, 70% to 99% supercoiled monomer, 70% to 99.5% supercoiled monomer, 70% to 99.9% supercoiled monomer, 75% to 80% supercoiled monomer, 75% to 90% supercoiled monomer, 75% to 95% supercoiled monomer, 75% to 99% supercoiled monomer, 75% to 99.5% supercoiled monomer, 75% to 99.9% supercoiled monomer, 80% to 85% supercoiled monomer, 80% to 90% supercoiled monomer, 80% to 95% supercoiled monomer, 80% to 99% supercoiled monomer, 80% to 99.5% supercoiled monomer, 80% to 99.9% supercoiled monomer, 85% to 90% supercoiled monomer, 85% to 95% supercoiled monomer, 85% to 99% supercoiled monomer, 85% to 99.5% supercoiled monomer, 85% to 99.9% supercoiled monomer, 90% to 95% supercoiled monomer, 90% to 99% supercoiled monomer, 90% to 99.5% supercoiled monomer, 90% to 99.9% supercoiled monomer, 95% to 99% supercoiled monomer, 95% to 99.5% supercoiled monomer, 95% to 99.9% supercoiled monomer, 98% to 99% supercoiled monomer, 98% to 99.5% supercoiled monomer, or 98% to 99.9% supercoiled monomer; e.g., about 60% supercoiled monomer, about 65% supercoiled monomer, about 70% supercoiled monomer, about 75% supercoiled monomer, about 80% supercoiled monomer, about 85% supercoiled monomer, about 90% supercoiled monomer, about 95% supercoiled monomer, about 96% supercoiled monomer, about 97% supercoiled monomer, about 98% supercoiled monomer, about 99% supercoiled monomer, or about 99.9% supercoiled monomer). In any of these instances, supercoiled monomer is calculated using densitometry analysis of gel electrophoresis.

In other embodiments, a pharmaceutical composition contains synthetic circular DNA vector that is not supercoiled (i.e., relaxed circular DNA), e.g., at least 50%, at least 60%, at least 70%, at least 80%, at least 90%, at least 95%, or 100% of the synthetic circular DNA vector in the pharmaceutical composition is not supercoiled.

In some embodiments, percent supercoiled monomer is determined by agarose gel electrophoresis or capillary electrophoresis. Additionally, or alternatively, percent supercoiled monomer is determined by anion exchange-HPLC.

In some embodiments, the pharmaceutical composition is substantially devoid of impurities. For instance, in some embodiments, the pharmaceutical composition contains <1.0% protein content by mass (e.g., <0.9%, <0.8%, <0.7%, <0.6%, <0.5%, <0.4%, <0.3%, <0.2%, <0.1%, <0.05%, or <0.01% protein content by mass), In some instances, protein content is determined by bicinchoninic acid assay. Additionally or alternatively, protein content is determined by ELISA.

In some instances, the pharmaceutical composition contains <1.0% RNA content by mass (e.g., <0.9%, <0.8%, <0.7%, <0.6%, <0.5%, <0.4%, <0.3%, <0.2%, <0.1%, <0.05%, or <0.01% RNA content by mass). In some embodiments, the RNA content is determined by agarose gel electrophoresis. In some embodiments, the RNA content is determined by quantitative PCR. In some embodiments, the RNA content is determined by fluorescence assay (e.g., Ribogreen).

In some embodiments, the pharmaceutical composition contains <1.0% gDNA content by mass (e.g., <0.9%, <0.8%, <0.7%, <0.6%, <0.5%, <0.4%, <0.3%, <0.2%, <0.1%, <0.05%, or <0.01% gDNA content by mass). In some embodiments, the gDNA content is determined by agarose gel electrophoresis or capillary electrophoresis. In some embodiments, the gDNA content is determined by quantitative PCR. In some embodiments, the gDNA content is determined by Southern blot.

In some embodiments, the pharmaceutical composition contains <40 EU/mg endotoxin. In some embodiments, the pharmaceutical composition contains <20 EU/mg endotoxin. In some embodiments, the pharmaceutical composition contains <10 EU/mg endotoxin. In some embodiments, the pharmaceutical composition contains <5 EU/mg endotoxin (e.g., <4 EU/mg endotoxin, <3 EU/mg endotoxin, <2 EU/mg endotoxin, <1 EU/mg endotoxin, <0.5 EU/mg endotoxin), e.g., as measured by Limulus Ameobocyte Lysate (LAL) assay.

Pharmaceutical compositions provided herein may include one or more pharmaceutically acceptable carriers, such as excipients and/or stabilizers that are nontoxic to the individual being treated (e.g., human patient) at the dosages and concentrations employed. In some embodiments, the pharmaceutically acceptable carrier is an aqueous pH buffered solution. Examples of pharmaceutically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as tween, polyethylene glycol (PEG), and pluronics.

If the pharmaceutical composition is provided in liquid form, the pharmaceutically acceptable carrier may be water (e.g., pyrogen-free water), isotonic saline, or a buffered aqueous solution, e.g., a phosphate buffered solution or a citrate buffered solution. Injection of the pharmaceutical composition may be carried out in water or a buffer, such as an aqueous buffer, e.g., containing a sodium salt (e.g., at least 50 mM of a sodium salt), a calcium salt (e.g., at least 0.01 mM of a calcium salt), or a potassium salt (e.g., at least 3 mM of a potassium salt). According to a particular embodiment, the sodium, calcium, or potassium salt may occur in the form of their halogenides, e.g., chlorides, iodides, or bromides, in the form of their hydroxides, carbonates, hydrogen carbonates, or sulfates, etc. Without being limited thereto, examples of sodium salts include NaCl, NaI, NaBr, Na₂CO₂, NaHCO₂, and Na₂SO₄. Examples of potassium salts include, e.g., KCl, KI, KBr, K₂CO₂, KHCO₂, and K₂SO₄. Examples of calcium salts include, e.g., CaCl₂, CaI₂, CaBr₂, CaCO₂, CaSO₄, and Ca(OH)₂. Additionally, organic anions of the aforementioned cations may be contained in the buffer. According to a particular embodiment, the buffer suitable for injection purposes as defined above, may contain salts selected from sodium chloride (NaCl), calcium chloride (CaCl₂) or potassium chloride (KCl), wherein further anions may be present. CaCl₂ can also be replaced by another salt, such as KCl. In some embodiments, salts in the injection buffer are present in a concentration of at least 50 mM sodium chloride (NaCl), at least 3 mM potassium chloride (KCl), and at least 0.01 mM calcium chloride (CaCl₂). The injection buffer may be hypertonic, isotonic, or hypotonic with reference to the specific reference medium, i.e., the buffer may have a higher, identical or lower salt content with reference to the specific reference medium, wherein preferably such concentrations of the afore mentioned salts may be used, which do not lead to damage of cells due to osmosis or other concentration effects. Reference media can be liquids such as blood, lymph, cytosolic liquids, other body liquids, or common buffers. Such common buffers or liquids are known to a skilled person. Ringer-Lactate solution is particularly preferred as a liquid basis.

One or more compatible solid or liquid fillers, diluents, or encapsulating compounds may be suitable for administration to a person. The constituents of the pharmaceutical composition according to the invention are capable of being mixed with the nucleic acid vector according to the invention as defined herein, in such a manner that no interaction occurs, which would substantially reduce the pharmaceutical effectiveness of the (pharmaceutical) composition according to the invention under typical use conditions. Pharmaceutically acceptable carriers, fillers and diluents can have sufficiently high purity and sufficiently low toxicity to make them suitable for administration to an individual being treated. Some examples of compounds which can be used as pharmaceutically acceptable carriers, fillers, or constituents thereof are sugars, such as lactose, glucose, trehalose, and sucrose; starches, such as corn starch of potato starch; dextrose; cellulose and its derivatives, such as sodium carboxymethylcellulose, ethylcellulose, cellulose acetate; powdered tragacanth; malt; gelatin; tallow; solid glidants, such as stearic acid, magnesium stearate; calcium sulfate; vegetable oils, such as groundnut oil, cottonseed oil, sesame oil, olive oil, corn oil and oil from theobroma; polyols, such as polypropylene glycol, glycerol, sorbitol, mannitol, and polyethylene glycol; or alginic acid.

The choice of a pharmaceutically acceptable carrier can be determined, according to the manner in which the pharmaceutical composition is administered.

Suitable unit dose forms for injection include sterile solutions of water, physiological saline, and mixtures thereof. The pH of such solutions may be adjusted to about 7.4. Suitable carriers for injection include hydrogels, devices for controlled or delayed release, polylactic acid, and collagen matrices. Suitable pharmaceutically acceptable carriers for topical application include those which are suitable for use in lotions, creams, gels and the like. If the pharmaceutical composition is to be administered perorally, tablets, capsules and the like are the preferred unit dose form.

Further additives which may be included in the pharmaceutical composition are emulsifiers, such as tween; wetting agents, such as sodium lauryl sulfate; coloring agents; pharmaceutical carriers; stabilizers; antioxidants; and preservatives.

The pharmaceutical composition according to the present invention may be provided in liquid or in dry (e.g., lyophilized) form. In a particular embodiment, the nucleic acid vector of the pharmaceutical composition is provided in lyophilized form. Lyophilized compositions including nucleic acid vector of the invention may be reconstituted in a suitable buffer, advantageously based on an aqueous carrier, prior to administration, e.g., Ringer-Lactate solution, Ringer solution, or a phosphate buffer solution.

In certain embodiments of the invention, any of the therapeutic circular DNA vectors of the invention can be complexed with one or more cationic or polycationic compounds, e.g., cationic or polycationic polymers, cationic or polycationic peptides or proteins, e.g., protamine, cationic of polycationic polysaccharides, and/or cationic or polycationic lipids.

According to a particular embodiment, a non-viral nucleic acid vector of the invention (e.g., a naked DNA vector, a circular DNA vector (e.g., a supercoiled circular DNA vector), and/or a synthetic DNA vector (e.g., a synthetic circular DNA vector) may be complexed with lipids to form one or more liposomes, lipoplexes, or lipid nanoparticles. Therefore, in one embodiment, the pharmaceutical composition comprises liposomes, lipoplexes, and/or lipid nanoparticles comprising any of the non-viral nucleic acid vectors of the invention. Such lipid-based delivery systems may be formulated for pulmonary delivery according to any suitable method known in the art or described herein.

Lipid-based formulations can be effective delivery systems for nucleic acid vectors due to their biocompatibility and their ease of large-scale production. Cationic lipids have been widely studied as synthetic materials for delivery of nucleic acids. After mixing together, nucleic acids are condensed by cationic lipids to form lipid/nucleic acid complexes known as lipoplexes. These lipid complexes are able to protect genetic material from the action of nucleases and deliver it into cells by interacting with the negatively charged cell membrane. Lipoplexes can be prepared by directly mixing positively charged lipids at physiological pH with negatively charged nucleic acids.

Conventional liposomes include of a lipid bilayer that can be composed of cationic, anionic, or neutral phospholipids and cholesterol, which encloses an aqueous core. Both the lipid bilayer and the aqueous space can incorporate hydrophobic or hydrophilic compounds, respectively. Liposome characteristics and behavior in vivo can be modified by addition of a hydrophilic polymer coating, e.g., polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains.

Liposomes are colloidal lipid-based and surfactant-based delivery systems composed of a phospholipid bilayer surrounding an aqueous compartment. They may present as spherical vesicles and can range in size from 20 nm to a few microns. Cationic lipid-based liposomes are able to complex with negatively charged nucleic acids via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the genetic material is then released from the endosome/carrier into the cytoplasm.

Cationic liposomes can serve as delivery systems for therapeutic circular DNA vectors. Cationic lipids, such as MAP, (1,2-dioleoyl-3-trimethylammonium-propane) and DOTMA (N-[1-(2,3-dioleoyloxy) propyl]-N,N,N-trimethyl-ammonium methyl sulfate) can form complexes or lipoplexes with negatively charged nucleic acids to form nanoparticles by electrostatic interaction, providing high in vitro transfection efficiency. Furthermore, neutral lipid-based nanoliposomes for nucleic acid vector delivery as e.g., neutral 1,2-dioleoyl-sn-glycero-3-phosphatidylcholine (DOPC)-based nanoliposomes are available.

Lipid nanoparticle (LNP) compositions and other lipid-based carriers shown to be suitable for delivery to the airway (e.g., by aerosol exposure) are contemplated as part of the present invention. For instance, any of the LNP compositions described in International Publication No.: WO 2022/104131, which is hereby incorporated by reference in its entirety, can be used to encapsulate any of the expression constructs or nucleic acid vectors described herein, e.g., for delivery to the target airway tissue, e.g., as an aerosolizable composition. Other known aerosolizable and/or nebulizable lipid compositions (e.g., lipid-nanovesicles) contemplated herein as adaptable for encapsulation and delivery of the nucleic acid vectors described herein include those described in, e.g., Kaur et al., Front Pharmacol. 2022 Mar. 10; 12:734913; Vartak et al, Nanomedicine, 2021 June; 16 (14): 1187-1202; Xu et al., Int. J. Nanomedicine. 2021 February 16:16:1221-1229; and Elhissi, Curr. Pharm. Des. 2017: 23 (3): 362-372.

Thus, in one embodiment of the invention, the therapeutic circular DNA vector of the invention is complexed with cationic lipids and/or neutral lipids and thereby forms liposomes, lipid nanoparticles, lipoplexes or neutral lipid-based nanoliposomes in the present pharmaceutical compositions.

In a particular embodiment, a pharmaceutical composition comprises the nucleic acid vector (e.g., nonviral DNA vector (e.g., naked DNA vector), circular DNA vector (e.g., supercoiled circular DNA vector), and/or synthetic DNA vector (e.g., synthetic circular DNA vector) of the invention that is formulated together with a cationic or polycationic compound and/or with a polymeric carrier. Accordingly, in a further embodiment of the invention, the nucleic acid vector (e.g., nonviral DNA vector (e.g., naked DNA vector), circular DNA vector (e.g., supercoiled circular DNA vector), and/or synthetic DNA vector (e.g., synthetic circular DNA vector)) is associated with or complexed with a cationic or polycationic compound or a polymeric carrier, optionally in a weight ratio selected from a range of about 5:1 (w/w) to about 0.25:1 (w/w), e.g., from about 5:1 (w/w) to about 0.5:1 (w/w), e.g., from about 4:1 (w/w) to about 1:1 (w/w) or of about 3:1 (w/w) to about 1:1 (w/w), e.g., from about 3:1 (w/w) to about 2:1 (w/w) of nucleic acid vector to cationic or polycationic compound and/or with a polymeric carrier; or optionally in a nitrogen/phosphate (N/P) ratio of nucleic acid vector to cationic or polycationic compound and/or polymeric carrier in the range of about 0.1-10, e.g., in a range of about 0.3-4 or 0.3-1, e.g., in a range of about 0.5-1 or 0.7-1, e.g., in a range of about 0.3-0.9 or 0.5-0.9. For example, the N/P ratio of the therapeutic circular DNA vector to the one or more polycations is in the range of about 0.1 to 10, including a range of about 0.3 to 4, of about 0.5 to 2, of about 0.7 to 2 and of about 0.7 to 1.5.

Pharmaceutical compositions may also involve association of the nucleic acid vectors described herein with a vehicle, transfection or complexation agent for increasing the transfection efficiency and/or the expression of the therapeutic gene according to the invention.

In some instances, the nucleic acid vector (e.g., nonviral DNA vector (e.g., naked DNA vector), circular DNA vector (e.g., supercoiled circular DNA vector), and/or synthetic DNA vector (e.g., synthetic circular DNA vector)) is complexed with one or more polycations, preferably with protamine or oligofectamine. Further cationic or polycationic compounds, which can be used as transfection or complexation agent may include cationic polysaccharides, for example chitosan, polybrene, cationic polymers, e.g., polyethyleneimine (PED), cationic lipids, e.g., DOTMA: [1-(2,3-sioleyloxy) propyl)]-N,N,N-trimethylammonium chloride, DMRIE, di-C14-amidine, DOTIM, SAINT, DC-Chol, BGTC, CTAP, DOPE, LEAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, MAP: dioleoyloxy-3-(trimethylammonio) propane, DC-6-14: O,O-ditetradecanoyl-N-(α-trimethylammonioacetyl) diethanolamine chloride, CLIP1: rac-[(2,3-dioctadecyloxypropyl) (2-hydroxyethyl)]-dimethylammonium chloride, CLIP6: rac-[2 (2,3-dihexadecyloxypropyl-oxymethyloxy)ethyl]trimethylammonium, CLIP9: rac-[2 (2,3-dihexadecyloxypropyl-oxysuccinyloxy)ethyl]-trimethylammonium, oligofectamine, or cationic or polycationic polymers, e.g., modified polyaminoacids, such as β-aminoacid-polymers or reversed polyamides, etc., modified polyethylenes, such as PVP (poly(N-ethyl-4-vinylpyridinium bromide), etc., modified acrylates, such as pDMAEMA (poly(dimethylaminoethyl methylacrylate)), etc., modified amidoamines such as pAMAM (poly(amidoamine)), etc., polybetaaminoester (PBAE) or modified PBAE (e.g., diamine end modified 1,4 butanediol diacrylate-co-5-amino-1-pentanol polymers, or polymers described in U.S. Pat. No. 8,557,231; PEGylated PBAE, such as those described in U.S. Patent Application No. 2018/0112038; any suitable polymer disclosed in Green et al., Acc. Chem. Res. 2008, 41 (6): 749-759, such as diamine end modified 1,4 butanediol diacrylate-co-5-amino-1-pentanol polymers; any suitable modified PBAE as described in International Patent Publication No. WO 2020/077159 or WO 2019/070727; PBAE 457 as disclosed in Shen et al., Soi. Adv. 2020, 6: eaba1606, etc.), dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., block polymers consisting of a combination of one or more cationic blocks (e.g., selected from a cationic polymer as mentioned above) and of one or more hydrophilic or hydrophobic blocks (e.g., polyethyleneglycol); etc.

In some instances, the pharmaceutical composition contains a nucleic acid vector (e.g., nonviral DNA vector (e.g., naked DNA vector), circular DNA vector (e.g., supercoiled circular DNA vector), and/or synthetic DNA vector (e.g., synthetic circular DNA vector) encapsulated in a nanoparticle or microparticle, e.g., a biodegradable nanoparticle or microparticle (e.g., a cationic biodegradable polymeric nanoparticle or microparticle, such as PBAE or a modified PBAE (such as a polymer of formula (I) of International Patent Publication No. WO 2019/070727, or PBAE 457 as disclosed in Shen et al., Sci. Adv. 2020, 6: eaba1606), a PEG-PBAE (or modified PBAE) copolymer), or a pH-sensitive nanoparticle or microparticle (e.g., a nanoparticle having a polymer of formula (I) of U.S. Pat. No. 10,792,374 (ECO).

According to a particular embodiment, the pharmaceutical composition includes the nucleic acid vector (e.g., nonviral ONA vector (e.g., naked DNA vector), circular DNA vector (e.g., supercoiled circular DNA vector), and/or synthetic DNA vector (e.g., synthetic circular DNA vector) encapsulated within or attached to a polymeric carrier (e.g., any of the aforementioned polymers described herein). A polymeric carrier used according to the invention may be a polymeric carrier formed by disulfide-crosslinked cationic components. The disulfide-crosslinked cationic components may be the same or different from each other. The polymeric carrier can also contain further components. It is also particularly preferred that the polymeric carrier used according to the present invention comprises mixtures of cationic peptides, proteins or polymers and optionally further components as defined herein, which are crosslinked by disulfide bonds as described herein. In this context, the disclosure of WO 2012/013326 is incorporated herein by reference. In this context, the cationic components that form basis for the polymeric carrier by disulfide-crosslinkage are typically selected from any suitable cationic or polycationic peptide, protein or polymer suitable for this purpose, particular any cationic or polycationic peptide, protein or polymer capable of complexing the nucleic acid vector as defined herein or a further nucleic acid comprised in the composition, and thereby preferably condensing the nucleic acid vector. The cationic or polycationic peptide, protein or polymer, may be a linear molecule; however, branched cationic or polycationic peptides, proteins or polymers may also be used.

Every disulfide-crosslinking cationic or polycationic protein, peptide or polymer of the polymeric carrier, which may be used to complex the nucleic acid vector according to the invention included as part of the pharmaceutical composition of the invention may contain at least one SH moiety (e.g., at least one cysteine residue or any further chemical group exhibiting an SH moiety) capable of forming a disulfide linkage upon condensation with at least one further cationic or polycationic protein, peptide or polymer as cationic component of the polymeric carrier as mentioned herein.

Such polymeric carriers used to complex the nucleic acid vector (e.g., nonviral DNA vector (e.g., naked DNA vector), circular DNA vector (e.g., supercoiled circular DNA vector), and/or synthetic DNA vector (e.g., synthetic circular DNA vector)) may be formed by disulfide-crosslinked cationic (of polycationic) components. In particular, such cationic or polycationic peptides or proteins or polymers of the polymeric carrier, which comprise or are additionally modified to comprise at least one SH moiety, can be selected from proteins, peptides, and polymers as a complexation agent.

In other embodiments, the nucleic acid vector (e.g., circular DNA vector (e.g., supercoiled circular DNA vector) and/or synthetic DNA vector (e.g., synthetic circular DNA vector) may be administered naked in a suitable buffer without being associated with any further vehicle, transfection, or complexation agent.

Any of the compositions described herein may be formulated in a suitable form for pulmonary administration, e.g., via the buccal cavity. Such a formulations may include dry particles which comprise the active ingredient and which have a diameter in the range from about 0.5 nm to about 7 nm or from about 1 nm to about 6 nm. Such compositions are suitably in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and active ingredient may constitute 0.1%> to 20% (w/w) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

Pharmaceutical compositions described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm.

Pharmaceutical compositions suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

### V. Methods of Use

Any of the vectors or pharmaceutical compositions described herein can be administered to a target cell (e.g., a target airway cell) in a subject in need thereof to express the transgene (e.g., a CFTR gene), e.g., for treatment of a respiratory disease, such as cystic fibrosis. Administration can be of any suitable route that results in a therapeutically effective outcome, e.g., pulmonary delivery. Routes of administration contemplated herein include nasal administration, insufflation, buccal, intrapulmonary, intrasinal, or respiratory (e.g., by inhaling orally or nasally). In some instances, the vector is administered in a particulate form (e.g., by lipid nanoparticle). Alternatively, the vector is administered as a naked vector (e.g., a naked DNA vector, e.g., a naked circular DNA vector). Whether administered as a particulate or naked formulation, the formulation can be aerosolized according to known methods.

In some instances, a naked vector is administered in combination with one or more procedures for electrotransfer. For example, such methods may include the following steps: (i) exposing the target airway cell to a nucleic acid vector, or pharmaceutical composition thereof, of the invention (e.g., a synthetic circular DNA vector), wherein the target airway cell is in electrical communication with an electrode; and (ii) transmitting one or more pulses of electrical energy through the electrode at conditions suitable for electrotransfer of the synthetic circular DNA vector into the target airway cell, thereby expressing the sequence of interest (e.g., a therapeutic sequence).

Conditions suitable for electrotransfer of the therapeutic agent into the target airway cell comprise a voltage at the target lung cell of less than 2,400 V. In some embodiments, the voltage at the target airway cell is between 10 V and 2,400 V, e.g., between 100 V and 2,400 V. e.g., from 100 V to 500 V, from 500 V to 1,000 V, from 1,000 V to 1,500 V, or from 1,500 V to 2,000 V. In some embodiments, 1-10 (e.g., 1-6) pulses of electrical energy are transmitted. In some embodiments, the total number of pulses of electrical energy are transmitted within 1-20 seconds. In some embodiments, a single pulse (i.e., one and only one pulse) of electrical energy is transmitted. In other embodiments, exactly two pulses of electrical energy is transmitted. In some embodiments, the one or more pulses of electrical energy are square waveforms. In some embodiments, the one or more pulses of electrical energy have an amplitude from 10 V to 10,000 V, e.g., from 100 V to 5,000 V, e.g., from 100 V to 500 V, from 500 V to 1,000 V, from 1,000 V to 1,500 V, or from 1,500 V to 2,000 V. In some embodiments, each of the pulses of electrical energy is from 0.01 to 200 milliseconds in duration. In some embodiments, each of the pulses of electrical energy is from 10 to 50 milliseconds in duration (e.g., about 20 milliseconds in duration). In some embodiments, the total duration of all of the pulses of electrical energy is from 10 to 50 milliseconds (e.g., about 20 milliseconds in total duration). In some embodiments, the electrode is a monopolar electrode. In other embodiments, the electrode is part of a bipolar electrode configuration. In some embodiments, the electrode is within 10 om of the target airway cell. For example, in some embodiments, the electrode is within 1 cm of the target airway cell (e.g., within 5 mm, within 4 mm, or within 3 mm).

A nucleic acid vector of the invention, or pharmaceutical composition thereof, can be administered within 24 hours of transmission of an electric field (e.g., within 20 hours, within 18 hours, within 16 hours, within 14 hours, within 12 hours, within 10 hours, within 8 hours, within 6 hours, within 4 hours, within 3 hours, within 2 hours, within 90 minutes, within 60 minutes, within 45 minutes, within 30 minutes, within 20 minutes, within 15 minutes, within 10 minutes, within 5 minutes, within 4 minutes, within 3 minutes, within 2 minutes, within 1 minute, within 45 seconds, within 30 seconds, within 20 seconds, within 15 seconds, within 10 seconds, or within 5 seconds preceding transmission of an electric field).

In some instances, the target airway cell is an airway epithelial cell. In some embodiments, the target airway cell is a lung cell. In some embodiments, the target airway cell is a nasal epithelial cell, a tracheal cell, bronchial cell, bronchiolar cell, or an alveolar cell. Step (ii) may be performed in situ in an individual by inserting a catheter comprising the electrode into an airway lumen of the individual. In some embodiments, the airway lumen is trachea, bronchi, or bronchiole. In some embodiments, the individual has been diagnosed with a disease or disorder. In some embodiments, the disease or disorder is a monogenic respiratory disease, e.g., cystic fibrosis.

Nucleic acid vectors or pharmaceutical compositions thereof can be administered to the individual locally (e.g., as part of an electrotransfer procedure). In some embodiments, the local administration is intranasal administration or intramuscular administration. In some embodiments, the local administration is by flood, spray, or aerosolization of the synthetic circular DNA vector. For administration by inhalation, the administering solution can be conveniently delivered in the form of an aerosol spray from a pressurized pack or a nebulizer, with the use of a suitable propellant, such as dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of gelatin, for example, for use in an inhaler or insufflator, can be formulated containing a powder mix of the molecule of interest and a suitable powder base such as lactose or starch.

A nucleic acid vector, or pharmaceutical composition thereof, can be administered within 24 hours of transmission of an electric field (e.g., within 20 hours, within 18 hours, within 16 hours, within 14 hours, within 12 hours, within 10 hours, within 8 hours, within 6 hours, within 4 hours, within 3 hours, within 2 hours, within 90 minutes, within 60 minutes, within 45 minutes, within 30 minutes, within 20 minutes, within 15 minutes, within 10 minutes, within 5 minutes, within 4 minutes, within 3 minutes, within 2 minutes, within 1 minute, within 45 seconds, within 30 seconds, within 20 seconds, within 15 seconds, within 10 seconds, or within 5 seconds preceding transmission of an electric field).

In some instances, a nucleic acid vector of the invention can be delivered to a target airway cell in an individual by: (i) inserting a catheter comprising a monopolar electrode into an airway lumen of the individual; (ii) positioning the electrode within a target region of the airway lumen comprising the target airway cell and the therapeutic agent; (iii) while the electrode is within the target region, transmitting one or more pulses of electrical energy through the electrode at conditions suitable for electrotransfer of the therapeutic agent into the target airway cell. In some embodiments, the individual has been treated with a partial airway epithelial ablation therapy. Partial airway epithelial ablation therapy can be performed using clinically available transbronchoscopic pulsed electric field system to remove columnar cells, thereby exposing basal stem cells in the bronchi.

In some instances, a nucleic acid vector, or pharmaceutical composition thereof, is delivered by a method that does not involve electrotransfer. Such alternative methods include delivery in a particle-based system, such as any particulate form described herein. In particular embodiments, the nucleic acid vector, or pharmaceutical composition thereof, is delivered to a target tissue or cell in a subject via lipid nanoparticles. Methods of delivering nucleic acid vectors by lipid nanoparticles (e.g., to respiratory tissues) are known in the art and readily adaptable to carry nucleic acid vectors described herein.

As a general proposition, an effective amount of a nucleic acid vector (e.g., synthetic circular DNA vector) administered to the individual (e.g., human) may be in the range from 1.0 μg to 1 mg of nucleic acid (e.g., from 0.01 ng to 100 μg, from 0.1 ng to 50 μg, from 1 ng to 10 μg, or from 10 ng to 1 μg, e.g., from 0.01 ng to 0.05 ng, from 0.05 ng to 0.1 ng, from 0.1 ng to 0.5 ng, from 0.5 ng to 1 ng, from 1 ng to 5 ng, from 5 ng to 10 ng, from 10 ng to 50 ng, from 50 ng to 100 ng, from 100 ng to 500 ng, from 500 ng to 1 μg, from 1 μg to 5 μg, or from 5 μg to 10 μg, e.g., about 1 μg, about 5 μg, about 10 μg, about 20 μg, about 25 μg, about 50 μg, about 75 μg, about 100 μg, about 1 ng, about 5 ng, about 10 ng, about 20 ng, about 25 ng, about 50 ng, about 75 ng, about 100 ng, about 1 ag, about 2 μg, about 3 μg, about 4 μg, about 5 μg, about 6 μg, about 7 μg, about 8 μg, about 9 ag, about 10 μg, about 15 μg, about 20 μg, about 25 μg, about 50 μg, about 75 μg, about 100 μg, or about 500 μg).

Dosages for a nucleic acid vector (e.g., circular DNA vector) or pharmaceutical composition thereof as described herein may be determined empirically in individuals who have been given one or more administrations of the nucleic acid vector (e.g., circular DNA vector) or pharmaceutical composition thereof. Individuals may be given incremental dosages of the therapeutic agent, e.g., nucleic acid vector (e.g., circular DNA vector) or pharmaceutical composition thereof. To assess efficacy of the nucleic acid vector (e.g., circular DNA vector) or pharmaceutical composition thereof, an indicator of the disease/disorder can be monitored. For repeated administrations over several days or longer, depending on the condition, the treatment is sustained until a desired result in the diseased or disorder is achieved.

In some instances, a single injection of the nucleic acid vector (e.g., circular DNA vector) or pharmaceutical composition thereof is administered to the individual over the course of the treatment. In some embodiments, the single injection of the nucleic acid vector (e.g., circular DNA vector) or pharmaceutical composition thereof is administered to the individual.

The therapeutic agent, e.g., nucleic acid vector (e.g., circular DNA vector) or pharmaceutical composition thereof can be administered as a single dose. Alternatively, the therapeutic agent, e.g., nucleic acid vector (e.g., circular DNA vector) or pharmaceutical composition thereof can be administered in multiple doses (e.g., two or more doses, three or more doses, four or more doses, five or more doses, six or more doses, e.g., two doses, three doses, four doses, five doses, or six doses) over the course of a treatment.

In some embodiments, dosing frequency is once every week, once every two weeks, once every three weeks, once every four weeks, once every five weeks, once every six weeks, once every seven weeks, once every eight weeks, once every nine weeks, or once every ten weeks; or once every month, once every two months, or once every three months, or less frequently. The progress of therapy can be readily monitored (e.g., detected or quantified), according to methods known in the art and described herein. The dosing regimen of the therapeutic agent, e.g., nucleic acid vector (e.g., circular DNA vector) or pharmaceutical composition thereof can vary over time.

In some embodiments, any of the pharmaceutical compositions described herein, when administered to a subject in need thereof, is sufficient to improve a measure of at least one respiratory volume by at least 10% (e.g., at least 15%, at least 20%, at least 25%, at least 30%, at least 40%, or at least 50%; e.g., from 10% to 100%, from 20% to 100%, from 30% to 100%, or from 40% to 100%), as compared to at least one reference respiratory volume measured in a subject untreated for cystic fibrosis. In some instances, the improvement is for at least 24 hours (e.g., at least 48 hours, at least 72 hours, at least 96 hours, at least 120 hours) after the first administration. In some instances, the respiratory volume (a measure of the amount of air inhaled, exhaled, and stored in the lungs) is total lung capacity, tidal volume, residual volume, expiratory reserve volume, inspiratory reserve volume, inspiratory capacity, inspiratory vital capacity, vital capacity, functional residual capacity, forced vital capacity, forced expiratory volume time, forced inspiratory flow, peak expiratory flow, or maximal voluntary ventilation.

### EXAMPLES

These examples are provided for illustrative purposes only and not to limit the scope of the claims provided herein.

### Example 1. Production of Synthetic Circular DNA Vectors Containing CFTR-S/MAR

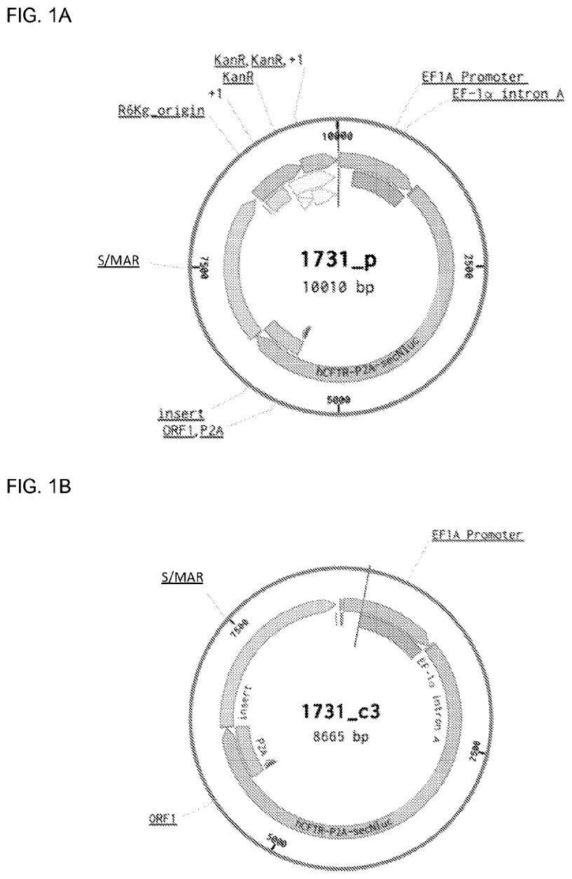

To produce a plasmid containing CFTR-S/MAR, shown in FIG. 1A, plasmids containing individual expression construct elements were first produced using standard manufacturing processes. Expression constructs included an EF1A promoter (SEQ ID NO: 8); a human CFTR sequence (SEQ ID NO: 1) with HA tag linked to secreted nanoluciferase through a P2A linker; and full-length human interferon beta S/MAR (SEQ ID NO: 2). These single-element plasmids were then ligated together to produce template plasmids containing all elements of an expression construct using a BsaI cloning process. Briefly, each plasmid having an individual expression element contained BsaI restriction sites flanking the element and overhangs required to ligate the sequences in the right order and orientation. The BsaI restriction reaction cut each required element from its respective plasmid and ligated the fragments into a new plasmid at the same time. The BsaI restriction overhangs were designed such that the ligation of the expression elements together would generate a plasmid having an expression construct with the elements in the order shown in FIG. 1A. The BsaI restriction and ligation reaction was prepared as follows: 2 μl of BSA buffer, 2 μl of T4 ligase buffer, 1.5 μl BsaI, 0.5 μl of T4 ligase, and equimolar concentrations of each plasmid preparation and water to reach 20 μl total volume. This master mix was then briefly vortexed/mixed and briefly centrifuged. The master mix was then placed in a thermocycler with the following steps: (1) 37° C. for 15 minutes; (2) 37° C. for two minutes; (3) 16° C. for five minutes; and (4) repeat steps (2) and (3) 50 times. One μl of the remaining product was then used to transform E. coli using protocols well known in the art. Resulting plasmids were then purified and DNA was digested with restriction enzyme to verify the accuracy of the final plasmid. Positive DNA sequence clones of interest were then verified using DNA sequencing and subsequently amplified following verification. These sequences were then cloned into a type IIs restriction site-containing backbone to form template plasmids for generation of synthetic circular DNA vectors.

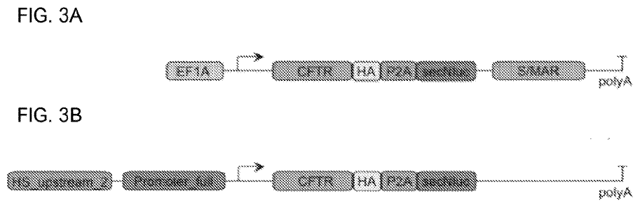

To produce the synthetic circular DNA vector of FIG. 1B from the template plasmid, methods generally taught in International Patent Publication Number WO 2019/178500 were used to remove plasmid backbone sequences (resistance genes (KanR) and bacterial origin of replication (R6 Kg origin)). Briefly, template plasmids were amplified by rolling circle amplification using Phi29 polymerase, restriction enzymes were added to cut the amplified product at sites flanking the therapeutic sequence, and the therapeutic sequence was recircularized by ligation using a ligase. In the present examples, the process was carried out using a single type IIs restriction enzyme, BsaI, which cut recognition sites flanking the therapeutic sequence and within the plasmid backbone. Upon ligation, the linear therapeutic fragment circularizes into a therapeutic circular DNA vector, and the linear backbone fragment circularizes. Without being bound by theory, the circularized backbone fragment contains a BsaI cut site and ligation occurs in the presence of the BsaI enzyme, so BsaI can cut the backbone and does not cut the therapeutic circular DNA vector, thereby driving the reaction forward toward a purer therapeutic circular DNA product. Exonuclease was added to digest the remaining linear backbone, and gyrase was added to supercoil the therapeutic circular DNA vector. The elements of the synthetic circular ONA vector of FIG. 1B are shown in linear form in FIG. 3A.

### Example 2. Production of Nucleic Acid Vectors Containing Hypersensitivity Sequences

Potential hypersensitivity sequences (HS) were assessed by analyzing genomic datasets with chromatin structure data deposited in publicly available repositories. Relevant datasets were chosen based on chromatin accessibility data, including histone post-translational modifications. Areas of enrichment for chromatin modification and/or open chromatin around CFTR were identified based on these data and potential regulatory elements were selected in sizes ranging from about two kilobases (kb) down to a few hundred bases. ChIP-Seq for H3K27ac and ATAC-Seq for open/accessible chromatin was performed, and candidate regulatory elements were mapped for CFTR expression construct generation. Selected candidate HS included HS1585 (SEQ ID NO: 11), HS1586 (SEQ ID NO: 12). HS1587 (SEQ ID NO: 13), or HS1588 (SEQ ID NO: 5).

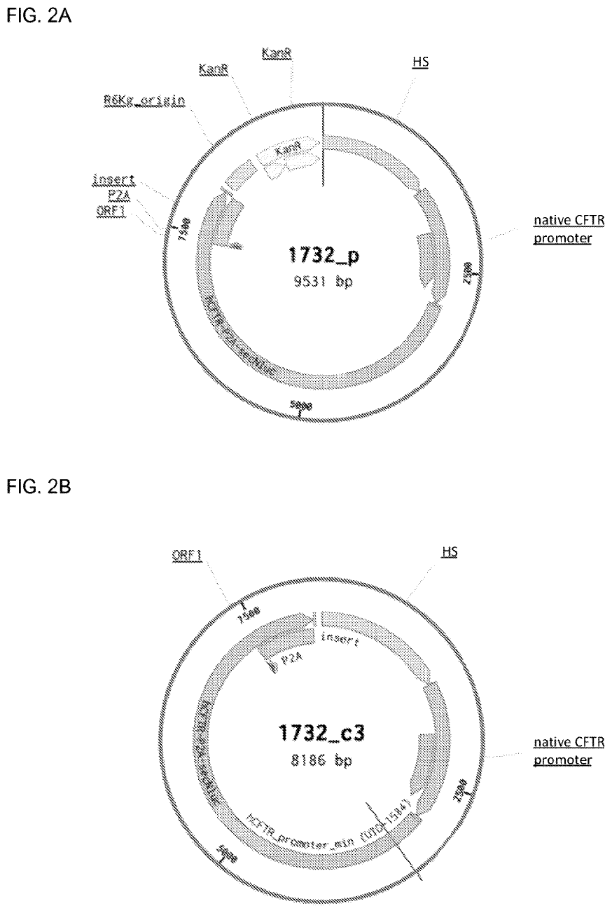

To produce a plasmids containing HS-CFTR, e.g., as shown in FIG. 2A, plasmids containing individual expression construct elements were first produced using standard manufacturing processes. In addition to the HS, expression constructs included a full-length native CFTR promoter (SEQ ID NO: 6); and a human CFTR sequence (SEQ ID NO: 1) with HA tag linked to secreted nanoluciferase (secNluc) through a P2A linker. These single-element plasmids were then ligated together to produce template plasmids containing all elements of an expression construct using a BsaI cloning process. Briefly, each plasmid having an individual expression element contained BsaI restriction sites flanking the element and overhangs required to ligate the sequences in the right order and orientation. The BsaI restriction reaction cut each required element from its respective plasmid and ligated the fragments into a new plasmid at the same time. The BsaI restriction overhangs were designed such that the ligation of the expression elements together would generate a plasmid having an expression construct with the elements in the order shown in FIG. 2A. The BsaI restriction and ligation reaction was prepared as follows: 2 μl of BSA buffer, 2 μl of T4 ligase buffer, 1.5 μl BsaI, 0.5 μl of T4 ligase, and equimolar concentrations of each plasmid preparation and water to reach 20 μl total volume. This master mix was then briefly vortexed/mixed and briefly centrifuged. The master mix was then placed in a thermocycler with the following steps: (1) 37° C. for 15 minutes; (2) 37° C. for two minutes; (3) 16° C. for five minutes; and (4) repeat steps (2) and (3) 50 times. One μl of the remaining product was then used to transform E. coli using protocols well known in the art. Resulting plasmids were then purified and DNA was digested with restriction enzyme to verify the accuracy of the final plasmid. Positive DNA sequence clones of interest were then verified using DNA sequencing and subsequently amplified following verification. These sequences were then cloned into a type IIs restriction site-containing backbone to form template plasmids for generation of synthetic circular DNA vectors.

To produce the synthetic circular DNA vector of FIG. 2B from the template plasmid, methods generally taught in International Patent Publication Number WO 2019/178500 were used to remove plasmid backbone sequences (resistance genes (KanR) and bacterial origin of replication (R6 Kg origin)). Briefly, template plasmids were amplified by rolling circle amplification using Phi29 polymerase, restriction enzymes were added to cut the amplified product at sites flanking the therapeutic sequence, and the therapeutic sequence was recircularized by ligation using a ligase. In the present examples, the process was carried out using a single restriction enzyme, BsaI, which cut recognition sites flanking the therapeutic sequence and within the plasmid backbone. Upon ligation, the linear therapeutic fragment circularizes into a therapeutic circular DNA vector, and the linear backbone fragment circularizes. Without being bound by theory, the circularized backbone fragment contains a BsaI cut site and ligation occurs in the presence of the BsaI enzyme, so BsaI can cut the backbone and does not cut the therapeutic circular DNA vector, thereby driving the reaction forward toward a purer therapeutic circular DNA product. Exonuclease was added to digest the remaining linear backbone, and gyrase was added to supercoil the therapeutic circular DNA vector. The elements of the synthetic circular DNA vector of FIG. 2B are shown in linear form in FIG. 3B.

### Example 3. CFTR Protein Expression by Plasmid DNA Vectors

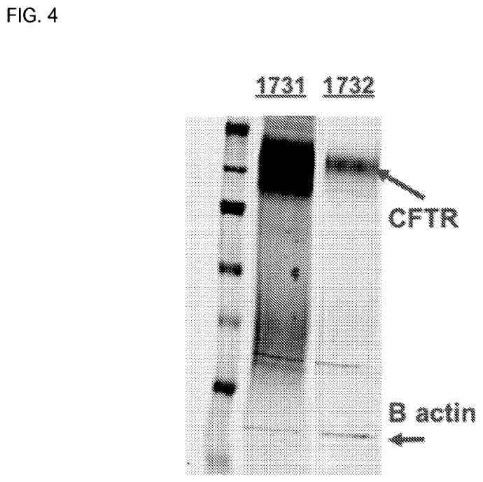

Plasmid DNA vectors containing different regulatory elements were compared for protein expression in HEK293T cells and A549 cells. Plasmids encoding CFTR and containing either a downstream S/MAR (p-1731; FIG. 1A) or an upstream hypersensitivity sequence (p-1732; FIG. 2A) were constructed as described in Examples 1 and 2, respectively, and tested by Western blot. Briefly, HEK293T were seeded at 200,000 cells per well pre-coated with poly-D-lysine and A549 cells were seeded at were seeded at the same density. Using a TRANSIT-X2® Dynamic Delivery System (Mirus), cells were transfected with plasmid DNA vectors and incubated for 72 hours. After incubation, cells were lysed and protein was measured using a standard BCA assay. Western blot was conducted using known methods. CFTR protein expression was observed for both plasmid constructs p-1731 and p-1732 in both HEK293T cells (FIG. 4) and A549 cells (data not shown).

### Example 4. CFTR Protein Expression by a Synthetic Circular DNA Vector Containing a Hypersensitivity Sequence

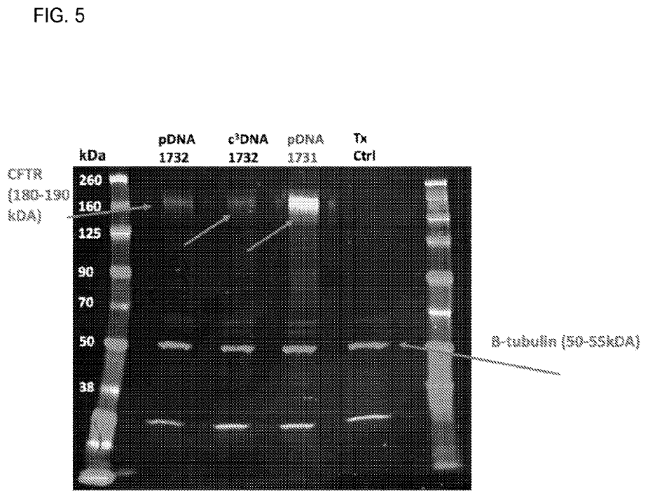

Synthetic circular DNA vector (C³) containing an upstream HS and encoding CFTR (shown in FIGS. 2B and 3B) were produced as described in Example 2 and tested by Western blot for protein expression in HEK293T cells in vitro. Plasmid DNA vectors containing the same therapeutic sequence (shown in FIG. 2A) and plasmid DNA vectors containing an S/MAR sequence (as shown in FIG. 1A) were also tested, Briefly, HEK293T cells were seeded at 200,000 cells per well of a 12-well plate coated with poly-D-lysine and transfected using a TRANSIT-X2@ Dynamic Delivery System (Mirus) according to standard protocol. 2 μg of each vector construct were transfected into HEK293T cells with lipofectamine 3000 (2 μL per well). Transfection controls were treated with Lipofectamine only. After four days, cells were lysed and Western blot was conducted using anti-CFTR antibody to evaluate presence of CFTR protein. Full-length CFTR protein expression was detected for all three vectors, which p1731 showing the highest expression (FIG. 5).

### Example 5. CFTR Protein Expression by Synthetic Circular DNA Vector Containing an S/MAR Sequence

Synthetic circular DNA vector (C³) encoding CFTR and containing a downstream S/MAR (shown in FIGS. 1B and 3A) were produced as described in Example 1 and tested by Western blot for protein expression in HEK293T cells in vitro in an experiment similar to that described in Example 4. Plasmid DNA vector containing the same therapeutic sequence (shown in FIG. 1A) was tested as a control.

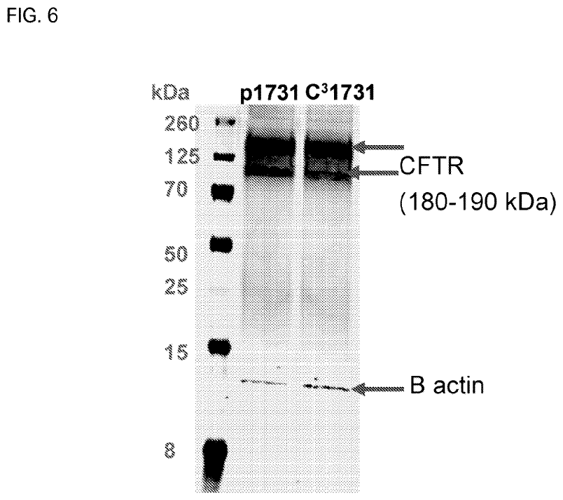

Both glycosylated and unglycosylated forms of CFTR protein were detected for both plasmid DNA and C³DNA vectors encoding CFTR and containing a downstream S/MAR (FIG. 6). These results confirm that a synthetic circular DNA vectors encoding CFTR and containing an S/MAR expression robustly express full-length CFTR protein.

### Example 6: Comparison of Electrotransfer-Mediated Expression by Synthetic Circular DNA Vectors with that of Plasmid DNA Vectors in Human Bronchial Epithelial Cells

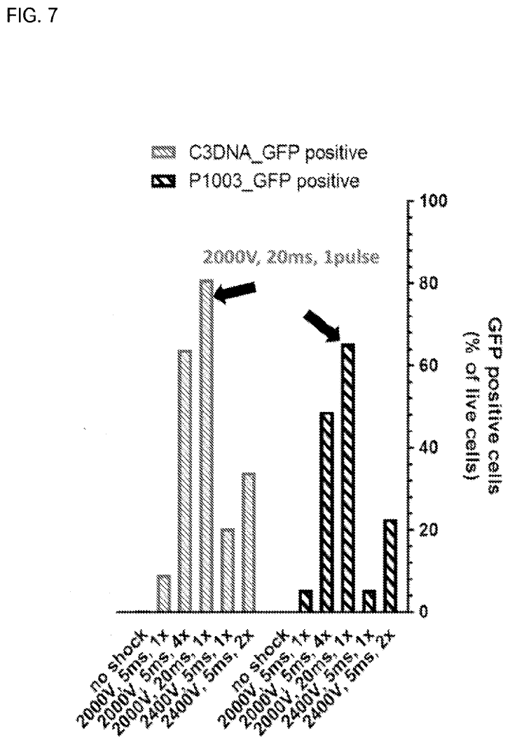

Primary human bronchial epithelial cells were suspended in culture media containing either synthetic circular DNA vector (c3DNA) encoding GFP or plasmid DNA vector (P1003) encoding GFP. Pulsed electric fields were applied at the conditions indicated in FIG. 7 (no shock; 2000 V/5 ms/1 pulse; 2000 V/5 ms/4 pulses; 2000 V/20 ms/1 pulse; 2400 V/5 ms/1 pulse; and 2400 V/5 ms/2 pulses) using the Neon Transfection System (Thermo Fisher Scientific). Cells were plated in wells after electroporation. GFP expression was assessed with flow cytometry 48 hours after transfection. GFP expression level was measured as a percent of live cells.

Synthetic circular DNA vectors showed greater expression levels of GFP compared to plasmid DNA vectors. Electroporation using conditions of 2000 V, 20 ms, 1 pulse showed the highest transfection efficiency in both synthetic circular DNA vectors and plasmid DNA vectors (FIG. 7). Additionally, increasing the pulse number at higher voltages, regardless of pulse length, decreased viability by ~50%.

### Example 7. Treatment of Cystic Fibrosis by Lipid Nanoparticle Delivery of CFTR-Encoding Vectors

A synthetic circular DNA vector containing CFTR-S/MAR, as shown in FIG. 18, is synthesized according to the methods described in Example 1.

The synthetic circular DNA vector is formulated into lipid nanoparticles (LNPs) according to known methods, e.g., as described in International Patent Publication No. WO 2022/104131. First, empty lipid nanoparticles are prepared by dissolving lipids in ethanol at a concentration of 40 mM and at an ionizable lipid:DSPC:cholesterol:DMG-PEG2K lipid ratio of 50.5:10.1:38.9:0.5 and mixed with 7.15 mM sodium acetate pH 5.0. Lipid solution and buffer are mixed using a multi-inlet vortex mixer at a 3.7 volumetric ratio of lipid:buffer. After a five-second residence time, empty LNPs are mixed with 5 mM sodium acetate pH 5.0 at a volumetric ratio of 5:7 of empty LNP:buffer. Dilute empty LNPs are then buffer exchanged and concentrated using tangential flow filtration into a final buffer containing 5 mM sodium acetate pH 5.0, and a sucrose solution is subsequently added to complete the storage matrix.

Synthetic circular DNA vector-loaded LNPs are prepared by mixing empty LNP at a lipid concentration of 2.85 mg/mL with synthetic circular DNA vector at a concentration of 0.25 mg/mL in 42.5 mM sodium acetate pH 5.0. N:P ratio is 4.93. Empty LNP solution and synthetic circular DNA vector are mixed using a multi-inlet vortex mixer at a 3:2 volumetric ratio of empty LNP:vector. Once the empty LNP are loaded with vector, they undergo a 30-60-second residence time prior to mixing in-line with a buffer containing 120 mM TRIS pH 8.12 at a volumetric ratio of 5:1 of LNP:buffer. Next, the LNP formulation was mixed in-line with a buffer containing 20 mM TRIS, 0.352 mg/ml DMG-PEG2k, 0.625 mg/mL GL-67, pH 7.5 at a volumetric ratio of 6:1 of LNP:buffer. The resulting LNP suspension is concentrated using tangential flow filtration and diluted with a salt solution to a final buffer matrix containing 70 mM NaCl. The resulting LNP suspension is filtered through 0.8/0.2 μm capsule filter and filled into glass vials.

A cystic fibrosis patient is determined to have a DeltaF508 mutation in CFTR. LNPs containing synthetic circular DNA vector are aerosolized according to known methods and administered to the patient by pulmonary delivery using a nebulizer. After the procedure, the patient is monitored weekly to assess disease progression using conventional methods.

### Example 8. Treatment of Cystic Fibrosis by Electrotransfer of Naked CFTR-Encoding Vectors

A cystic fibrosis patient is determined to have a DeltaF508 mutation in CFTR. A synthetic circular DNA vector containing HS-CFTR, as shown in FIG. 2B, is synthesized according to the methods described in Example 2. The synthetic circular DNA vector is formulated in a nebulizer with dichlorodifluoromethane, trichlorofluoromethane, dichlorotetrafluoroethane, carbon dioxide or other suitable gas and administered by inhalation to the patient.

Within five to thirty minutes from inhalation of the synthetic circular DNA vector encoding CFTR, a catheter having an electrode on its distal terminus is progressed through the patient's airway by a physician. The physician positions the electrode in the bronchial lumen and administers two 20 ms pulses of amplitude 250 V. The electrode is removed from the patient, and the procedure is complete. After the procedure, the patient is monitored weekly to assess disease progression using conventional methods.

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

SEQUENCE TABLE

| SEQ ID | |
|---|---|
| NO: | |
| 1 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| 2 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| 3 | [symbol] |
| 4 | [symbol] |
| 5 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| 6 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| 7 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| 8 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| 9 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| 10 | [symbol] |
| | [symbol] |
| | [symbol] |
| | [symbol] |
| | [symbol] |
| | [symbol] |
| | [symbol] |
| 11 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| 12 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| 13 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| 14 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| 15 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| 16 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| 17 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |

[symbol]

indicates data missing or illegible when filed

What is claimed is:

1. A circular DNA vector comprising a promoter, a transgene, and a scaffold matrix attachment region (S/MAR) sequence, wherein the transgene is a therapeutic protein-encoding sequence derived from a protein expressed in respiratory epithelium, and wherein the circular DNA vector lacks a drug resistance gene.

2. (canceled)

3. (canceled)

4. The circular DNA vector of claim 1, wherein the therapeutic protein is a cystic fibrosis transmembrane receptor (CFTR), wherein the CFTR is at least 95% identical to SEQ ID NO: 1.

5. (canceled)

6. The circular DNA vector of claim 1, wherein the S/MAR sequence is an interferon-beta S/MAR sequence or a functional variant thereof.

7. (canceled)

8. (canceled)

9. The circular DNA vector of claim 1, wherein the promoter is operably linked 5′ to the transgene, and the S/MAR sequence is operably linked 3′ to the transgene.

10. (canceled)

11. (canceled)

12. (canceled)

13. (canceled)

14. (canceled)

15. The circular DNA vector of claim 1, wherein the 3′ end of the S/MAR sequence is connected to the 5′ end of the promoter by a non-bacterial sequence of less than 500 bp, wherein the non-bacterial sequence connecting the 3′ end of the S/MAR sequence to the 5′ end of the promoter corresponds to a restriction enzyme cut site overhang.

16. (canceled)

17. (canceled)

18. A nucleic acid vector comprising a hypersensitivity sequence operably linked 5′ to a transgene, wherein the hypersensitivity sequence comprises SEQ ID NO: 5, or a functional variant thereof.

19. (canceled)

20. (canceled)

21. (canceled)

22. The nucleic acid vector of claim 18, wherein the transgene is a therapeutic protein-encoding sequence derived from a protein expressed in respiratory epithelium, wherein the therapeutic protein is a cystic fibrosis transmembrane receptor (CFTR), wherein the CFTR is at least 95% identical to SEQ ID NO: 1.

23. (canceled)

24. (canceled)

25. (canceled)

26. (canceled)

27. The nucleic acid vector of claim 1, further comprising a promoter operably linked 5′ to the transgene, wherein the promoter is operably linked 3′ to the hypersensitivity sequence.

28. (canceled)

29. (canceled)

30. (canceled)

31. (canceled)

32. The nucleic acid vector of claim 18, wherein the transgene is operatively linked to a reporter sequence.

33. (canceled)

34. The nucleic acid vector of claim 18, wherein the nucleic acid vector is a plasmid DNA vector, a minicircle DNA vector, a nanoplasmid DNA vector, a synthetic circular DNA vector, a closed-ended DNA vector, a doggybone DNA vector, or a ministring DNA vector.

35. The nucleic acid vector of claim 18, wherein the nucleic acid vector is a circular DNA vector that lacks a drug resistance gene.

36. (canceled)

37. (canceled)

38. (canceled)

39. (canceled)

40. (canceled)

41. The nucleic acid vector of claim 18, wherein the 3′ end of the transgene is connected to the 5′ end of the hypersensitivity sequence by a non-bacterial sequence, wherein the non-bacterial sequence connecting the 3′ end of the transgene to the 5′ end of the hypersensitivity sequence corresponds to a restriction enzyme cut site overhang.

42. A pharmaceutical composition comprising a therapeutically effective amount of the circular DNA vector of claim 1, or the nucleic acid vector of claim 18, and a pharmaceutically acceptable carrier.

43. (canceled)

44. (canceled)

45. The pharmaceutical composition of claim 42, wherein the circular DNA vector or nucleic acid vector is formulated as a liposomal or nanoparticulate formulation.

46. The pharmaceutical composition of claim 45, wherein the circular DNA vector or nucleic acid vector is formulated for respiratory delivery.


SEQUENCE LISTING (17 sequences)

SEQ ID NO: 1 - DNA, 4446 bp, Synthetic construct
  source 1..4446: mol_type = other DNA
gccaccatgcagagaagccctctggaaaaggccagcgtggtgtccaagctgttcttcagctggacccggcctatcctgcggaagggctatagacagagactggaactgagcgacatctatcagatccccagcgtggacagcgccgacaacctgtctgagaagctggaaagagagtgggacagagagctggcctccaagaagaaccccaagctgatcaacgccctgcggcggtgcttcttctggcggtttatgttctacggcatcttcctgtacctgggcgaagtgaccaaagccgtgcagcctctgctgctgggcagaatcattgccagctacgaccccgacaacaaagaggaacggtctatcgccatctacctcggcatcggcctgtgcctgctgtttatcgtcagaaccctgctgctgcaccccgccatctttggactgcaccacatcggcatgcagatgcggatcgccatgttcagcctgatctacaagaaaaccctgaagctgtccagcagagtgctggacaagatcagcatcggacagctggtgtccctgctgagcaacaacctgaacaagttcgacgaaggcctggctctggcccacttcgtgtggattgctcctctgcaagtggccctgctgatgggcctgatttgggaactgctgcaggccagcgccttttgcggactgggatttctgattgtgctggccctgttccaggccggactggggagaatgatgatgaagtaccgggaccagagagccggcaagatctccgagagactggtcatcaccagcgagatgatcgagaacatccagagcgtgaaggcctactgctgggaagaggccatggaaaagatgattgagaatctgcggcagaccgagctgaagctgacaagaaaggccgcctacgtgcgctacttcaacagcagcgccttcttcttctccggcttcttcgtggtgttcctgagcgtgctgccctacgctctgatcaagggcatcatcctgagaaagattttcaccaccatttctttctgcatcgtgctgcggatggccgtgaccagacagtttccttgggctgtgcagacttggtacgatagcctgggcgccatcaacaagatccaggacttcctgcagaagcaagagtacaagaccctcgagtacaacctgaccaccaccgaggtggtcatggaaaacgtgaccgccttctgggaggaaggcttcggcgagctgtttgagaaggccaagcagaacaacaacaaccgcaagaccagcaacggcgacgacagcctgttcttctccaacttctccctgctggggacccctgtgctgaaggacatcaacttcaagatcgagcggggacagctgctggccgtggctggatctacaggcgccggaaaaaccagcctgctcatggtcatcatgggcgagctggaacctagcgagggcaagatcaagcacagcggcaggatcagcttctgtagccagttctcctggatcatgcccggcaccatcaaagagaacatcatcttcggcgtgtcctacgacgagtacagataccgcagcgtgatcaaggcctgccagctggaagaggacatcagcaagttcgccgagaaggacaacatcgtgctcggcgaaggcggcatcacactgtctggcggacagagggccagaatctctctggctagagccgtgtacaaggacgccgatctgtacctgctggatagccccttcggctacctggatgtgctgaccgagaaagagatcttcgagagctgcgtgtgcaagctgatggccaacaagacccggatcctggtcacctccaagatggaacacctgaagaaggccgacaagatcctgattctgcacgagggcagcagctacttttacggcaccttcagcgagctgcagaacctgcagcctgacttcagcagcaaactgatgggctgcgactccttcgaccagttcagcgccgagcggagaaacagcatcctgacagagacactgcaccggttctccctggaaggcgacgctcctgtgtcttggaccgagacaaagaagcagagcttcaagcagaccggcgagtttggcgagaagcggaagaactccattctgaaccccatcaactccatccggaagttcagcatcgtccagaaaacccctctgcagatgaacggcatcgaagaggatagcgacgagcccctggaaagacggctgtctctggtgcctgatagcgaacagggcgaagccatcctgcctcggatctccgtgattagcacaggccctacactgcaggctcggagaaggcagagtgtgctgaacctgatgacccacagcgtgaaccagggacagaatatccacagaaagaccaccgccagcacacggaaagtgtcactggcccctcaggccaacctgactgagctggacatctacagcagacggctgagccaagagacaggcctggaaatcagcgaggaaatcaacgaagaggacctgaaagagtgctttttcgacgacatggaatctatccccgccgtgacaacctggaatacctacctgcggtacatcaccgtgcacaagtccctgatcttcgtgctgatctggtgtctcgtgatcttcctggccgaagtggccgcttctctggtggttctgtggctgctcggaaacaccccactgcaggacaagggcaatagcacccacagccggaacaacagctacgccgtgatcatcacctccaccagctcctactacgtgttctacatctacgtgggcgtcgccgacactctgctcgccatgggattctttagaggactgcccctggtgcacaccctgatcaccgtgtctaagatcctgcaccataagatgctgcacagcgtcctgcaggcccctatgagcacactgaacaccctgaaagccggcggaatcctgaacagattcagcaaggacattgccatcctggacgacctgctgcctctgaccatcttcgacttcatccagctgctgctgatcgtgatcggcgccattgctgtggtggctgtgctgcagccttacatcttcgtggccaccgtgcctgtgatcgtggccttcattatgctgcgggcctactttctgcagacctctcagcagctgaagcagctcgagtctgagggcagaagccccatctttacccacctcgtgaccagcctgaaaggcctgtggaccctgagagcctttggcagacagccctacttcgagacactgttccacaaggccctgaacctgcacaccgccaactggtttctgtatctgagcaccctgcggtggttccagatgaggatcgagatgattttcgtcatcttctttatcgccgtgaccttcatcagcatcctcaccactggcgaaggcgagggcagagtgggaatcattctgaccctggccatgaacatcatgtccacactccagtgggccgtgaacagcagcatcgatgtggacagcctgatgcggagcgtgtcccgggtgttcaagttcatcgacatgcccacagagggcaagcccaccaagagcaccaagccttacaagaacggccagctgagcaaagtcatgatcatcgagaactcccacgtcaagaaggacgacatttggcccagcggaggccagatgaccgtgaaggatctgaccgccaagtacaccgaaggcggaaacgccattctggaaaacatcagctttagcatcagccctggccagcgcgtgggactccttggaagaaccggaagcggcaagtctactctgctgagcgccttcctgagactgctgaataccgagggcgagatccagatcgatggggtgtcctgggacagcatcaccctgcaacaatggcggaaggcctttggcgtgatccctcagaaggtgttcattttcagcggcacgttccggaagaatctggacccctacgagcagtggagcgaccaagagatttggaaggtggccgatgaagtgggactgagaagcgtgatcgagcagtttcccggcaagctggatttcgtgctggtggatggcggctgtgtgctgtctcacggacacaagcagctgatgtgcctggccagatccgtgctgtccaaggccaagattctgctgctcgacgagcctagcgctcacctggatcctgtgacctaccagatcatccggcggacactgaagcaggcctttgccgattgcaccgtgatcctgtgcgagcacagaattgaggccatgctggaatgccagcagtttctggttatcgaagagaacaaagtgcggcagtacgacagcatccagaagctgctgaacgagcggagcctgttcagacaggccatctctcccagcgacagagtgaagctgttccctcaccggaacagctccaagtgcaagagcaagcctcagatcgccgctctgaaagaagaaaccgaggaagaggtgcaggacacacggctg

SEQ ID NO: 2 - DNA, 2210 bp, Synthetic construct
  source 1..2210: mol_type = other DNA
gaattcagcaaggtcgccacgcacaagatcaatattaacaatcagtcatctctctttagcaataaaaaggtgaaaaattacattttaaaaatgacaccatagacgatgtatgaaaataatctacttggaaataaatctaggcaaagaagtgcaagactgttacccagaaaacttacaaattgtaaatgagaggttagtgaagatttaaatgaatgaagatctaaataaacttataaattgtgagagaaattaatgaatgtctaagttaatgcagaaacggagagacatactatattcatgaactaaaagacttaatattgtgaaggtatactttcttttcacataaatttgtagtcaatatgttcaccccaaaaaagctgtttgttaacttgtcaacctcatttcaaaatgtatatagaaagcccaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagtcattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgattgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagtggaatgatttaggaagtattccctctgcttctgtcttctgaaagagattgtagaaagttgatacaatttttttttctttaaatatcttgatagaattcataa

SEQ ID NO: 3 - DNA, 20 bp, Synthetic construct
  source 1..20: mol_type = other DNA
gtatcacaggagaatttcag

SEQ ID NO: 4 - DNA, 28 bp, Synthetic construct
  source 1..28: mol_type = other DNA
ttcggtatcacaggagaatttcagggag

SEQ ID NO: 5 - DNA, 1419 bp, Synthetic construct
  source 1..1419: mol_type = other DNA
ctctgctgcccaggctggaatgcagtggcatgaacactgttcactgcagcctcgacctcctgagctcaagcaatcctcccacctcagcctcctgagtagctgggactacaggtgcatgccactatgcaggctaattttaaaatttgttgagatggattctcgttgcattgcccaggctgatctcaaacttctggtctaaaatgaacctcctgcctcagcctcccgaagtggtgagattataggcataagccactgcaccaggctagtgattgctaacacttaaatagcacttccaagtgccttgacacttttctaaacactttgtattcactaattcgtgtaattctcactatagttttttaaggtaaatactatcgttatctccattttatagatgaggaaactgaggaacaggaagatcatataaactgcccaaggtcatagatgtggaacattctgaactaacaccagactattgggccctatggtggtgctcagatgttacttggaaagtataatgctcacagaagcactctctctctctaggggaatacttgtttccatcatttgttttactctctgtagagggaaacattcctttccaaaggagatttcatcttctgactagtaaatcagactcagaatgagctatgattcatttacaggtaagtcacaagctttattcctggccagataagaaatcagtaatttggttttgtctgaagagatttgggaattcccccaagatgatttcctggagtgggtcagaatgccacctacacactaggttaatgacttcagtgttctgcttggttcgctgatgacaggaaactggtaaaacaaagagggtgagaaaccagctgtatcacagtaataaatattactcagatttaggatcttcttcctagttcctgagtttttttaactttcaagctttcactcagacaatatgcaatgattagctaatacagtttgttttagtattacaaatgatgatggcagattttgcggggaatttgttttaaagttaatgttatcatatttttacaattcttgttttcaatagtttgaaaaagttctgttgggatcctgaattcttaattttgactgagatctagtgagattagttgtctcttttggagataattttagaggcataaaaatggtaaatcaattttttttcaattattagaaatcatgtgcacaaaatagtcaaggggaaagattagcaactgcttttttaatatagcattaattgatttcacaaatagttactatgctgctactgtattttttaggcatcagagatagagttgtgaggaagacaaggcaagatccttgacctccaaatgtttatagtctagcaagataaattaataaatatataattaagtaataggtaatgataaattctataaa

SEQ ID NO: 6 - DNA, 1492 bp, Synthetic construct
  source 1..1492: mol_type = other DNA
gccttagatgtgtcggcaataaagtaatcaggccaaaatttttactttcctttgaatttttcaattcaaacacaatgtatgcttgcttttacacagtagggttcagggattagagggttggctctttaaaaaccgtcagagacacaggcaatcctacacaaaattctcagaaggaaggcgcctacgcctgggaatgcccagatgcccctcagagagttgaagatggcgtttctctgagtcaggtcaaagttaacacattaccttcgcttcaaagactgcttggcttcctttcggtggattagtcaagatgttttgctgactgagactaggaaatctataggagggcgggttagtttacattgttccttgtcattatcgctaaaacactccaaagccttccttaaaaatgcgcactgggctaaaaaggatagacaaggaacacatcctgggccggtaattacgcaaagcattatctcctcttacctccttgcagatttttttttctctttcagtacgtgtcctaagatttctgtgccacccttggagttcactcacctaaacctgaaactaataaagcttggttcttttctccgacacgcaaaggaagcgctaaggtaaatgcatcagacccacactgccgcggaacttttcggctctctaaggctgtattttgatatacgaaaggcacattttccttcccttttcaaaatgcaccttgcaaacgtaacaggaacccgactaggatcatcgggaaaaggaggaggaggaggaaggcaggctccggggaagctggtggcagcgggtcctgggtctggcggaccctgacgcgaaggagggtctaggaagctctccggggagccggttctcccgccggtggcttcttctgtcctccagcgttgccaactggacctaaagagaggccgcgactgtcgcccacctgcgggatgggcctggtgctgggcggtaaggacacggacctggaaggagcgcgcgcgagggagggaggctgggagtcagaatcgggaaagggaggtgcggggcggcgagggagcgaaggaggagaggaggaaggagcgggaggggtgctggcgggggtgcgtagtgggtggagaaagccgctagagcaaatttggggccggaccaggcagcactcggcttttaacctgggcagtgaaggcgggggaaagagcaaaaggaaggggtggtgtgcggagtaggggtgggtggggggaattggaagcaaatgacatcacagcaggtcagagaaaaagggttgagcggcaggcacccagagtagtaggtctttggcattaggagcttgagcccagacggccctagcagggaccccagcgcccgagatacccagaggtcgcctctggaaaaggccagcgttgtctccaaactttttttcaggtgagaaggtggccaaccgagcttcggaaagacacgtgcccacgaaagaggagggcgtgtgtatgggttgggtttgggg

SEQ ID NO: 7 - DNA, 787 bp, Synthetic construct
  source 1..787: mol_type = other DNA
ggagttcactcacctaaacctgaaactaataaagcttggttcttttctccgacacgcaaaggaagcgctaaggtaaatgcatcagacccacactgccgcggaacttttcggctctctaaggctgtattttgatatacgaaaggcacattttccttcccttttcaaaatgcaccttgcaaacgtaacaggaacccgactaggatcatcgggaaaaggaggaggaggaggaaggcaggctccggggaagctggtggcagcgggtcctgggtctggcggaccctgacgcgaaggagggtctaggaagctctccggggagccggttctcccgccggtggcttcttctgtcctccagcgttgccaactggacctaaagagaggccgcgactgtcgcccacctgcgggatgggcctggtgctgggcggtaaggacacggacctggaaggagcgcgcgcgagggagggaggctgggagtcagaatcgggaaagggaggtgcggggcggcgagggagcgaaggaggagaggaggaaggagcgggaggggtgctggcgggggtgcgtagtgggtggagaaagccgctagagcaaatttggggccggaccaggcagcactcggcttttaacctgggcagtgaaggcgggggaaagagcaaaaggaaggggtggtgtgcggagtaggggtgggtggggggaattggaagcaaatgacatcacagcaggtcagagaaaaagggttgagcggcaggcacccagagtagtaggtctttggcattaggagcttgagcccaga

SEQ ID NO: 8 - DNA, 1179 bp, Synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 9 - DNA, 1681 bp, Synthetic construct
  source 1..1681: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacagggagtttaaa

SEQ ID NO: 10 - DNA, 540 bp, Synthetic construct
  source 1..540: mol_type = other DNA
gcaggcatgcttctatattattttctaaaagatttaaagttttgccttctccatttagacttataattcactggaatttttttgtgtgtatggtatgacatatgggttcccttttattttttacatataaatatatttccctgtttttctaaaaaagaggggatcctctagagtcgacctgcaggcatgcttctatattattttctaaaagatttaaagttttgccttctccatttagacttataattcactggaatttttttgtgtgtatggtatgacatatgggttcccttttattttttacatataaatatatttccctgtttttctaaaaaagaggggatcctctagagtcgacctgcaggcatgcttctatattattttctaaaagatttaaagttttgccttctccatttagacttataattcactggaatttttttgtgtgtatggtatgacatatgggttcccttttattttttacatataaatatatttccctgtttttctaaaaaagaggggatcctctagagtcgacct

SEQ ID NO: 11 - DNA, 1514 bp, Synthetic construct
  source 1..1514: mol_type = other DNA
tggagaaggtggaatcacactgagtggaggtcaacgagcaagaatttctttagcaaggtgaataactaattattggtctagcaagcatttgctgtaaatgtcattcatgtaaaaaaattacagacatttctctattgctttatattctgtttctggaattgaaaaaatcctggggttttatggctagtgggttaagaatcacatttaagaactataaataatggtatagtatccagatttggtagagattatggttactcagaatctgtgcccgtatcttggtgtcagtgtatttgtttgcctcatagtatagtttactacaaatggaaaactctaggattctgcataatactggacagagaagatgtaaatatctgttagttccatcatagaccctgccactccaatgtacacaccagctttaggcttcttggtatagataaacatacattttcaaaatttttcatcataattttcataacaaaataggaaggcaaatgatgtcacttggcttaaaatctataatatttaaaataaacaggacaaatgcattaacattgttgggggaggaggtcccttagtagaaacactcttggtccaagcattttaaagctgtcaaagagatgtaaatatagataatgtatgtcaaggagagagctttgtggttaaactgtaactttcagtttaaacaattattggtgactctgatgtcaaatgtttctcaagctttatctgaacaaaattcttctcactttgttgccaaagtcgttaacaagaaatcacattgactcattgatgttttggctcctttcccttactttctgttgctttccaaaagctgagacaggaaactaaccctaactgagcacctgcaattgcctggtagtattctagtcatgtgtgtacttttgtgtgtatgtaatccccttacagctctgcaaagtaagaattgttctccctgctttacagaagagatcataagataattgaggctgttagatgttaacttgccaaaagccatacaggaaaatggtagagtcacagtttgaaccaggtccttttgattctttacattaaaccatgctttgatcttggaaatacactgtaaggcaataaatcaatagatacggataattcacaggcttctaaataaatggaagttgattgtttttatctgtgagccaaagtaagacttattctaagaattccacaaatttagataagatagagtatatggcttctagacatccaacatagaactgagtttgtgttatcagtttaagatttggttttgctgtaaggtgcacacactttgaggaactaaaaataattgtctgttcttattctgatcagaatgtgtaatgtgttgtccagttttggatgatgaatttcttatttctaatctcataagaaacttgtcatagatgtgagggagagaattaagaacagagtgtggggaagaaactgtgtacattttgatgggatccattatgtagctcttgcatactgtcttc

SEQ ID NO: 12 - DNA, 767 bp, Synthetic construct
  source 1..767: mol_type = other DNA
gctggtattacaggtgtacaccactacgcccaggtaattttgtgtttttagtagagacagggtttcgccatgttggccaggctggtcttgaagtcctgacctcatgatccacctgcctcagcctcccaaagttctgggtttacaagcgtgagccacagtgcctggccaagaatcttatcttaatcctctgtcttaagacaatttatcctggaaaaatgattatccattttcttcaagtctctctccataaaacctctttatggaatctccttttgatttgaactttgatccaaatcataaacaatcctcattccctcttaatgttatgtatcacggatgtgagactgggtgtataccggtgtatatgtgggggaacagtggtgtcctgaatgccctttagacctgatctttatgaatcaccatgatatttctatttcctatgacctgtgtgatttttggttgttacttatcttgacaaatattcttttcaaaaacattgcgcactgaaggacatctggaaaattccaggagtctgtctaggttctaattgagatgcaatttcctaccttcatagccttttattgggcaatgtttgttgacacttgttttccaagttactggattactcatttcagagttcagttacccagaaaccacctacttctatgcctacaataagaagcaaacaagaggtttttgcaataaagacaatcagtatctaggataagggctggcatgtgggctggtcccatctttgctgtgaagct

SEQ ID NO: 13 - DNA, 1479 bp, Synthetic construct
  source 1..1479: mol_type = other DNA
cttaggtacctattgctattctcctttagcacaccatcaaccctctttactgttataggatcttagtcttgtgtattaaccctctactgcctgcccaaccaccaccaccaagtgtctcatatcctcacaaacaagtttggtcagctactgcaagggttcaaccatttttctctttctttccattctagttcccttatgctaagtgatatgacttctgcaagttcttccatatctttttggcatggctgctgctctcatgagaatcacatactcaccccaccacttgggctttttcactaagtaccaccactgagttgctggagactaggcaattgcacagacatctgtaaaaataggcaacaatgtctttattgtgtcatgctctgctggaaggggacccaaaaatatttccttacttgtggagtttcgccaaggacaatgtcttattgaaagaaaccatatcattcattaaatcattcttgccttctagccatttagcattttttaaagcatctatttactgagcacatttatcagcaccagccttagtgctgtatatgcattattttctttttatccttgaaataaccttgagaaacaagggttattctcctcaattttatagataaggaacccaagagcagagaacttaaatacttgccaggtgtcacataggtaatagctatgtggtgattaaaagccagaacaatgaaactctgtagaagatgatgtgtggctatatctaccttaccctgctgtccatttcatcctccagctcatatgactgtgggctgataattcagagagttactctcttctgagagactatgtttcttccaattcctaccagcagatcctgaccaatgactcaatgatagagagttacaaagggctagccacttgcttcaaggtagggctaattctgtggcgcaatttgtgctccagagcattcctgtgggatcaaactgaaactagtctctaactgagccacattcttgcctctcctgacctatcctacttccttcacttttcttcttttgagagtacagcccagtaaatcacttggacaagaaaactcatctcatgctttcccaggaaacctgacttaaaccagacccatgatccaattttttaagtaccacagtacactgtcctcttttttttttttataagaccaagaggatttactcaattatttctttccttactatgaagtgcctcaactgctcacgtaaatgggtagtacaatacagtaaatctgtatgatgaatgcatcttgtgatcgggttcggttgttcttacactctattcttttcatggacagtattctaaagttgaacatagataaatatttgtaatcagtcttgtgaacttctggcccaaggatttctagtggaaattttagattgtatgcctttgaatagaaataagtttaatgcttcacatttttttctgatagtaaaaattgcaaaattagaa

SEQ ID NO: 14 - DNA, 10010 bp, Synthetic construct
  source 1..10010: mol_type = other DNA
ttcggtatcacaggagaatttcagggagcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacagataagtttgtacaaaaaagagaggtgccaccatgcagagaagccctctggaaaaggccagcgtggtgtccaagctgttcttcagctggacccggcctatcctgcggaagggctatagacagagactggaactgagcgacatctatcagatccccagcgtggacagcgccgacaacctgtctgagaagctggaaagagagtgggacagagagctggcctccaagaagaaccccaagctgatcaacgccctgcggcggtgcttcttctggcggtttatgttctacggcatcttcctgtacctgggcgaagtgaccaaagccgtgcagcctctgctgctgggcagaatcattgccagctacgaccccgacaacaaagaggaacggtctatcgccatctacctcggcatcggcctgtgcctgctgtttatcgtcagaaccctgctgctgcaccccgccatctttggactgcaccacatcggcatgcagatgcggatcgccatgttcagcctgatctacaagaaaaccctgaagctgtccagcagagtgctggacaagatcagcatcggacagctggtgtccctgctgagcaacaacctgaacaagttcgacgaaggcctggctctggcccacttcgtgtggattgctcctctgcaagtggccctgctgatgggcctgatttgggaactgctgcaggccagcgccttttgcggactgggatttctgattgtgctggccctgttccaggccggactggggagaatgatgatgaagtaccgggaccagagagccggcaagatctccgagagactggtcatcaccagcgagatgatcgagaacatccagagcgtgaaggcctactgctgggaagaggccatggaaaagatgattgagaatctgcggcagaccgagctgaagctgacaagaaaggccgcctacgtgcgctacttcaacagcagcgccttcttcttctccggcttcttcgtggtgttcctgagcgtgctgccctacgctctgatcaagggcatcatcctgagaaagattttcaccaccatttctttctgcatcgtgctgcggatggccgtgaccagacagtttccttgggctgtgcagacttggtacgatagcctgggcgccatcaacaagatccaggacttcctgcagaagcaagagtacaagaccctcgagtacaacctgaccaccaccgaggtggtcatggaaaacgtgaccgccttctgggaggaaggcttcggcgagctgtttgagaaggccaagcagaacaacaacaaccgcaagaccagcaacggcgacgacagcctgttcttctccaacttctccctgctggggacccctgtgctgaaggacatcaacttcaagatcgagcggggacagctgctggccgtggctggatctacaggcgccggaaaaaccagcctgctcatggtcatcatgggcgagctggaacctagcgagggcaagatcaagcacagcggcaggatcagcttctgtagccagttctcctggatcatgcccggcaccatcaaagagaacatcatcttcggcgtgtcctacgacgagtacagataccgcagcgtgatcaaggcctgccagctggaagaggacatcagcaagttcgccgagaaggacaacatcgtgctcggcgaaggcggcatcacactgtctggcggacagagggccagaatctctctggctagagccgtgtacaaggacgccgatctgtacctgctggatagccccttcggctacctggatgtgctgaccgagaaagagatcttcgagagctgcgtgtgcaagctgatggccaacaagacccggatcctggtcacctccaagatggaacacctgaagaaggccgacaagatcctgattctgcacgagggcagcagctacttttacggcaccttcagcgagctgcagaacctgcagcctgacttcagcagcaaactgatgggctgcgactccttcgaccagttcagcgccgagcggagaaacagcatcctgacagagacactgcaccggttctccctggaaggcgacgctcctgtgtcttggaccgagacaaagaagcagagcttcaagcagaccggcgagtttggcgagaagcggaagaactccattctgaaccccatcaactccatccggaagttcagcatcgtccagaaaacccctctgcagatgaacggcatcgaagaggatagcgacgagcccctggaaagacggctgtctctggtgcctgatagcgaacagggcgaagccatcctgcctcggatctccgtgattagcacaggccctacactgcaggctcggagaaggcagagtgtgctgaacctgatgacccacagcgtgaaccagggacagaatatccacagaaagaccaccgccagcacacggaaagtgtcactggcccctcaggccaacctgactgagctggacatctacagcagacggctgagccaagagacaggcctggaaatcagcgaggaaatcaacgaagaggacctgaaagagtgctttttcgacgacatggaatctatccccgccgtgacaacctggaatacctacctgcggtacatcaccgtgcacaagtccctgatcttcgtgctgatctggtgtctcgtgatcttcctggccgaagtggccgcttctctggtggttctgtggctgctcggaaacaccccactgcaggacaagggcaatagcacccacagccggaacaacagctacgccgtgatcatcacctccaccagctcctactacgtgttctacatctacgtgggcgtcgccgacactctgctcgccatgggattctttagaggactgcccctggtgcacaccctgatcaccgtgtctaagatcctgcaccataagatgctgcacagcgtcctgcaggcccctatgagcacactgaacaccctgaaagccggcggaatcctgaacagattcagcaaggacattgccatcctggacgacctgctgcctctgaccatcttcgacttcatccagctgctgctgatcgtgatcggcgccattgctgtggtggctgtgctgcagccttacatcttcgtggccaccgtgcctgtgatcgtggccttcattatgctgcgggcctactttctgcagacctctcagcagctgaagcagctcgagtctgagggcagaagccccatctttacccacctcgtgaccagcctgaaaggcctgtggaccctgagagcctttggcagacagccctacttcgagacactgttccacaaggccctgaacctgcacaccgccaactggtttctgtatctgagcaccctgcggtggttccagatgaggatcgagatgattttcgtcatcttctttatcgccgtgaccttcatcagcatcctcaccactggcgaaggcgagggcagagtgggaatcattctgaccctggccatgaacatcatgtccacactccagtgggccgtgaacagcagcatcgatgtggacagcctgatgcggagcgtgtcccgggtgttcaagttcatcgacatgcccacagagggcaagcccaccaagagcaccaagccttacaagaacggccagctgagcaaagtcatgatcatcgagaactcccacgtcaagaaggacgacatttggcccagcggaggccagatgaccgtgaaggatctgaccgccaagtacaccgaaggcggaaacgccattctggaaaacatcagctttagcatcagccctggccagcgcgtgggactccttggaagaaccggaagcggcaagtctactctgctgagcgccttcctgagactgctgaataccgagggcgagatccagatcgatggggtgtcctgggacagcatcaccctgcaacaatggcggaaggcctttggcgtgatccctcagaaggtgttcattttcagcggcacgttccggaagaatctggacccctacgagcagtggagcgaccaagagatttggaaggtggccgatgaagtgggactgagaagcgtgatcgagcagtttcccggcaagctggatttcgtgctggtggatggcggctgtgtgctgtctcacggacacaagcagctgatgtgcctggccagatccgtgctgtccaaggccaagattctgctgctcgacgagcctagcgctcacctggatcctgtgacctaccagatcatccggcggacactgaagcaggcctttgccgattgcaccgtgatcctgtgcgagcacagaattgaggccatgctggaatgccagcagtttctggttatcgaagagaacaaagtgcggcagtacgacagcatccagaagctgctgaacgagcggagcctgttcagacaggccatctctcccagcgacagagtgaagctgttccctcaccggaacagctccaagtgcaagagcaagcctcagatcgccgctctgaaagaagaaaccgaggaagaggtgcaggacacacggctgggcggcagctacccttacgatgtgcctgattatgccggcagcggcgctacaaactttagccttctgaagcaggccggggatgtcgaagagaatcctggtccaatgaacagcttcagcaccagcgcctttggacccgtggccttttctctgggactgctgctggttctgcctgccgcttttcctgctcctgtgttcaccctggaagatttcgtcggcgattggagacagaccgccggctacaatctggaccaggtgttggagcaaggcggcgtgtccagcctgtttcagaacctgggagtgtccgtgacacccatccagagaatcgtgctgagcggcgagaacggcctgaagatcgacatccacgtgatcatcccttacgagggcctgtccggcgatcagatgggacagatcgagaagatctttaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacggcaacaagatcatcgacgagcggctgatcaaccccgatggcagcctgctgttcagagtgaccatcaacggcgtgacaggatggcggctgtgcgagagaattctggcctaagcttgaattcagcaaggtcgccacgcacaagatcaatattaacaatcagtcatctctctttagcaataaaaaggtgaaaaattacattttaaaaatgacaccatagacgatgtatgaaaataatctacttggaaataaatctaggcaaagaagtgcaagactgttacccagaaaacttacaaattgtaaatgagaggttagtgaagatttaaatgaatgaagatctaaataaacttataaattgtgagagaaattaatgaatgtctaagttaatgcagaaacggagagacatactatattcatgaactaaaagacttaatattgtgaaggtatactttcttttcacataaatttgtagtcaatatgttcaccccaaaaaagctgtttgttaacttgtcaacctcatttcaaaatgtatatagaaagcccaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagtcattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgattgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagtggaatgatttaggaagtattccctctgcttctgtcttctgaaagagattgtagaaagttgatacaatttttttttctttaaatatcttgatagaattcataaaataaaatatctttattttcattacatctgtgtgttggttttttgtgtgcgctactaacctgagaccgatctgttgatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacaggtctcaaaccctc

SEQ ID NO: 15 - DNA, 8665 bp, Synthetic construct
  source 1..8665: mol_type = other DNA
ttcggtatcacaggagaatttcagggagcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacagataagtttgtacaaaaaagagaggtgccaccatgcagagaagccctctggaaaaggccagcgtggtgtccaagctgttcttcagctggacccggcctatcctgcggaagggctatagacagagactggaactgagcgacatctatcagatccccagcgtggacagcgccgacaacctgtctgagaagctggaaagagagtgggacagagagctggcctccaagaagaaccccaagctgatcaacgccctgcggcggtgcttcttctggcggtttatgttctacggcatcttcctgtacctgggcgaagtgaccaaagccgtgcagcctctgctgctgggcagaatcattgccagctacgaccccgacaacaaagaggaacggtctatcgccatctacctcggcatcggcctgtgcctgctgtttatcgtcagaaccctgctgctgcaccccgccatctttggactgcaccacatcggcatgcagatgcggatcgccatgttcagcctgatctacaagaaaaccctgaagctgtccagcagagtgctggacaagatcagcatcggacagctggtgtccctgctgagcaacaacctgaacaagttcgacgaaggcctggctctggcccacttcgtgtggattgctcctctgcaagtggccctgctgatgggcctgatttgggaactgctgcaggccagcgccttttgcggactgggatttctgattgtgctggccctgttccaggccggactggggagaatgatgatgaagtaccgggaccagagagccggcaagatctccgagagactggtcatcaccagcgagatgatcgagaacatccagagcgtgaaggcctactgctgggaagaggccatggaaaagatgattgagaatctgcggcagaccgagctgaagctgacaagaaaggccgcctacgtgcgctacttcaacagcagcgccttcttcttctccggcttcttcgtggtgttcctgagcgtgctgccctacgctctgatcaagggcatcatcctgagaaagattttcaccaccatttctttctgcatcgtgctgcggatggccgtgaccagacagtttccttgggctgtgcagacttggtacgatagcctgggcgccatcaacaagatccaggacttcctgcagaagcaagagtacaagaccctcgagtacaacctgaccaccaccgaggtggtcatggaaaacgtgaccgccttctgggaggaaggcttcggcgagctgtttgagaaggccaagcagaacaacaacaaccgcaagaccagcaacggcgacgacagcctgttcttctccaacttctccctgctggggacccctgtgctgaaggacatcaacttcaagatcgagcggggacagctgctggccgtggctggatctacaggcgccggaaaaaccagcctgctcatggtcatcatgggcgagctggaacctagcgagggcaagatcaagcacagcggcaggatcagcttctgtagccagttctcctggatcatgcccggcaccatcaaagagaacatcatcttcggcgtgtcctacgacgagtacagataccgcagcgtgatcaaggcctgccagctggaagaggacatcagcaagttcgccgagaaggacaacatcgtgctcggcgaaggcggcatcacactgtctggcggacagagggccagaatctctctggctagagccgtgtacaaggacgccgatctgtacctgctggatagccccttcggctacctggatgtgctgaccgagaaagagatcttcgagagctgcgtgtgcaagctgatggccaacaagacccggatcctggtcacctccaagatggaacacctgaagaaggccgacaagatcctgattctgcacgagggcagcagctacttttacggcaccttcagcgagctgcagaacctgcagcctgacttcagcagcaaactgatgggctgcgactccttcgaccagttcagcgccgagcggagaaacagcatcctgacagagacactgcaccggttctccctggaaggcgacgctcctgtgtcttggaccgagacaaagaagcagagcttcaagcagaccggcgagtttggcgagaagcggaagaactccattctgaaccccatcaactccatccggaagttcagcatcgtccagaaaacccctctgcagatgaacggcatcgaagaggatagcgacgagcccctggaaagacggctgtctctggtgcctgatagcgaacagggcgaagccatcctgcctcggatctccgtgattagcacaggccctacactgcaggctcggagaaggcagagtgtgctgaacctgatgacccacagcgtgaaccagggacagaatatccacagaaagaccaccgccagcacacggaaagtgtcactggcccctcaggccaacctgactgagctggacatctacagcagacggctgagccaagagacaggcctggaaatcagcgaggaaatcaacgaagaggacctgaaagagtgctttttcgacgacatggaatctatccccgccgtgacaacctggaatacctacctgcggtacatcaccgtgcacaagtccctgatcttcgtgctgatctggtgtctcgtgatcttcctggccgaagtggccgcttctctggtggttctgtggctgctcggaaacaccccactgcaggacaagggcaatagcacccacagccggaacaacagctacgccgtgatcatcacctccaccagctcctactacgtgttctacatctacgtgggcgtcgccgacactctgctcgccatgggattctttagaggactgcccctggtgcacaccctgatcaccgtgtctaagatcctgcaccataagatgctgcacagcgtcctgcaggcccctatgagcacactgaacaccctgaaagccggcggaatcctgaacagattcagcaaggacattgccatcctggacgacctgctgcctctgaccatcttcgacttcatccagctgctgctgatcgtgatcggcgccattgctgtggtggctgtgctgcagccttacatcttcgtggccaccgtgcctgtgatcgtggccttcattatgctgcgggcctactttctgcagacctctcagcagctgaagcagctcgagtctgagggcagaagccccatctttacccacctcgtgaccagcctgaaaggcctgtggaccctgagagcctttggcagacagccctacttcgagacactgttccacaaggccctgaacctgcacaccgccaactggtttctgtatctgagcaccctgcggtggttccagatgaggatcgagatgattttcgtcatcttctttatcgccgtgaccttcatcagcatcctcaccactggcgaaggcgagggcagagtgggaatcattctgaccctggccatgaacatcatgtccacactccagtgggccgtgaacagcagcatcgatgtggacagcctgatgcggagcgtgtcccgggtgttcaagttcatcgacatgcccacagagggcaagcccaccaagagcaccaagccttacaagaacggccagctgagcaaagtcatgatcatcgagaactcccacgtcaagaaggacgacatttggcccagcggaggccagatgaccgtgaaggatctgaccgccaagtacaccgaaggcggaaacgccattctggaaaacatcagctttagcatcagccctggccagcgcgtgggactccttggaagaaccggaagcggcaagtctactctgctgagcgccttcctgagactgctgaataccgagggcgagatccagatcgatggggtgtcctgggacagcatcaccctgcaacaatggcggaaggcctttggcgtgatccctcagaaggtgttcattttcagcggcacgttccggaagaatctggacccctacgagcagtggagcgaccaagagatttggaaggtggccgatgaagtgggactgagaagcgtgatcgagcagtttcccggcaagctggatttcgtgctggtggatggcggctgtgtgctgtctcacggacacaagcagctgatgtgcctggccagatccgtgctgtccaaggccaagattctgctgctcgacgagcctagcgctcacctggatcctgtgacctaccagatcatccggcggacactgaagcaggcctttgccgattgcaccgtgatcctgtgcgagcacagaattgaggccatgctggaatgccagcagtttctggttatcgaagagaacaaagtgcggcagtacgacagcatccagaagctgctgaacgagcggagcctgttcagacaggccatctctcccagcgacagagtgaagctgttccctcaccggaacagctccaagtgcaagagcaagcctcagatcgccgctctgaaagaagaaaccgaggaagaggtgcaggacacacggctgggcggcagctacccttacgatgtgcctgattatgccggcagcggcgctacaaactttagccttctgaagcaggccggggatgtcgaagagaatcctggtccaatgaacagcttcagcaccagcgcctttggacccgtggccttttctctgggactgctgctggttctgcctgccgcttttcctgctcctgtgttcaccctggaagatttcgtcggcgattggagacagaccgccggctacaatctggaccaggtgttggagcaaggcggcgtgtccagcctgtttcagaacctgggagtgtccgtgacacccatccagagaatcgtgctgagcggcgagaacggcctgaagatcgacatccacgtgatcatcccttacgagggcctgtccggcgatcagatgggacagatcgagaagatctttaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacggcaacaagatcatcgacgagcggctgatcaaccccgatggcagcctgctgttcagagtgaccatcaacggcgtgacaggatggcggctgtgcgagagaattctggcctaagcttgaattcagcaaggtcgccacgcacaagatcaatattaacaatcagtcatctctctttagcaataaaaaggtgaaaaattacattttaaaaatgacaccatagacgatgtatgaaaataatctacttggaaataaatctaggcaaagaagtgcaagactgttacccagaaaacttacaaattgtaaatgagaggttagtgaagatttaaatgaatgaagatctaaataaacttataaattgtgagagaaattaatgaatgtctaagttaatgcagaaacggagagacatactatattcatgaactaaaagacttaatattgtgaaggtatactttcttttcacataaatttgtagtcaatatgttcaccccaaaaaagctgtttgttaacttgtcaacctcatttcaaaatgtatatagaaagcccaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagtcattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgattgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagtggaatgatttaggaagtattccctctgcttctgtcttctgaaagagattgtagaaagttgatacaatttttttttctttaaatatcttgatagaattcataaaataaaatatctttattttcattacatctgtgtgttggttttttgtgtgcgctactaaccctc

SEQ ID NO: 16 - DNA, 9531 bp, Synthetic construct
  source 1..9531: mol_type = other DNA
ttcgctctgctgcccaggctggaatgcagtggcatgaacactgttcactgcagcctcgacctcctgagctcaagcaatcctcccacctcagcctcctgagtagctgggactacaggtgcatgccactatgcaggctaattttaaaatttgttgagatggattctcgttgcattgcccaggctgatctcaaacttctggtctaaaatgaacctcctgcctcagcctcccgaagtggtgagattataggcataagccactgcaccaggctagtgattgctaacacttaaatagcacttccaagtgccttgacacttttctaaacactttgtattcactaattcgtgtaattctcactatagttttttaaggtaaatactatcgttatctccattttatagatgaggaaactgaggaacaggaagatcatataaactgcccaaggtcatagatgtggaacattctgaactaacaccagactattgggccctatggtggtgctcagatgttacttggaaagtataatgctcacagaagcactctctctctctaggggaatacttgtttccatcatttgttttactctctgtagagggaaacattcctttccaaaggagatttcatcttctgactagtaaatcagactcagaatgagctatgattcatttacaggtaagtcacaagctttattcctggccagataagaaatcagtaatttggttttgtctgaagagatttgggaattcccccaagatgatttcctggagtgggtcagaatgccacctacacactaggttaatgacttcagtgttctgcttggttcgctgatgacaggaaactggtaaaacaaagagggtgagaaaccagctgtatcacagtaataaatattactcagatttaggatcttcttcctagttcctgagtttttttaactttcaagctttcactcagacaatatgcaatgattagctaatacagtttgttttagtattacaaatgatgatggcagattttgcggggaatttgttttaaagttaatgttatcatatttttacaattcttgttttcaatagtttgaaaaagttctgttgggatcctgaattcttaattttgactgagatctagtgagattagttgtctcttttggagataattttagaggcataaaaatggtaaatcaattttttttcaattattagaaatcatgtgcacaaaatagtcaaggggaaagattagcaactgcttttttaatatagcattaattgatttcacaaatagttactatgctgctactgtattttttaggcatcagagatagagttgtgaggaagacaaggcaagatccttgacctccaaatgtttatagtctagcaagataaattaataaatatataattaagtaataggtaatgataaattctataaaggaggccttagatgtgtcggcaataaagtaatcaggccaaaatttttactttcctttgaatttttcaattcaaacacaatgtatgcttgcttttacacagtagggttcagggattagagggttggctctttaaaaaccgtcagagacacaggcaatcctacacaaaattctcagaaggaaggcgcctacgcctgggaatgcccagatgcccctcagagagttgaagatggcgtttctctgagtcaggtcaaagttaacacattaccttcgcttcaaagactgcttggcttcctttcggtggattagtcaagatgttttgctgactgagactaggaaatctataggagggcgggttagtttacattgttccttgtcattatcgctaaaacactccaaagccttccttaaaaatgcgcactgggctaaaaaggatagacaaggaacacatcctgggccggtaattacgcaaagcattatctcctcttacctccttgcagatttttttttctctttcagtacgtgtcctaagatttctgtgccacccttggagttcactcacctaaacctgaaactaataaagcttggttcttttctccgacacgcaaaggaagcgctaaggtaaatgcatcagacccacactgccgcggaacttttcggctctctaaggctgtattttgatatacgaaaggcacattttccttcccttttcaaaatgcaccttgcaaacgtaacaggaacccgactaggatcatcgggaaaaggaggaggaggaggaaggcaggctccggggaagctggtggcagcgggtcctgggtctggcggaccctgacgcgaaggagggtctaggaagctctccggggagccggttctcccgccggtggcttcttctgtcctccagcgttgccaactggacctaaagagaggccgcgactgtcgcccacctgcgggatgggcctggtgctgggcggtaaggacacggacctggaaggagcgcgcgcgagggagggaggctgggagtcagaatcgggaaagggaggtgcggggcggcgagggagcgaaggaggagaggaggaaggagcgggaggggtgctggcgggggtgcgtagtgggtggagaaagccgctagagcaaatttggggccggaccaggcagcactcggcttttaacctgggcagtgaaggcgggggaaagagcaaaaggaaggggtggtgtgcggagtaggggtgggtggggggaattggaagcaaatgacatcacagcaggtcagagaaaaagggttgagcggcaggcacccagagtagtaggtctttggcattaggagcttgagcccagacggccctagcagggaccccagcgcccgagatacccagaggtcgcctctggaaaaggccagcgttgtctccaaactttttttcaggtgagaaggtggccaaccgagcttcggaaagacacgtgcccacgaaagaggagggcgtgtgtatgggttgggtttggggcagataagtttgtacaaaaaagagaggtgccaccatgcagagaagccctctggaaaaggccagcgtggtgtccaagctgttcttcagctggacccggcctatcctgcggaagggctatagacagagactggaactgagcgacatctatcagatccccagcgtggacagcgccgacaacctgtctgagaagctggaaagagagtgggacagagagctggcctccaagaagaaccccaagctgatcaacgccctgcggcggtgcttcttctggcggtttatgttctacggcatcttcctgtacctgggcgaagtgaccaaagccgtgcagcctctgctgctgggcagaatcattgccagctacgaccccgacaacaaagaggaacggtctatcgccatctacctcggcatcggcctgtgcctgctgtttatcgtcagaaccctgctgctgcaccccgccatctttggactgcaccacatcggcatgcagatgcggatcgccatgttcagcctgatctacaagaaaaccctgaagctgtccagcagagtgctggacaagatcagcatcggacagctggtgtccctgctgagcaacaacctgaacaagttcgacgaaggcctggctctggcccacttcgtgtggattgctcctctgcaagtggccctgctgatgggcctgatttgggaactgctgcaggccagcgccttttgcggactgggatttctgattgtgctggccctgttccaggccggactggggagaatgatgatgaagtaccgggaccagagagccggcaagatctccgagagactggtcatcaccagcgagatgatcgagaacatccagagcgtgaaggcctactgctgggaagaggccatggaaaagatgattgagaatctgcggcagaccgagctgaagctgacaagaaaggccgcctacgtgcgctacttcaacagcagcgccttcttcttctccggcttcttcgtggtgttcctgagcgtgctgccctacgctctgatcaagggcatcatcctgagaaagattttcaccaccatttctttctgcatcgtgctgcggatggccgtgaccagacagtttccttgggctgtgcagacttggtacgatagcctgggcgccatcaacaagatccaggacttcctgcagaagcaagagtacaagaccctcgagtacaacctgaccaccaccgaggtggtcatggaaaacgtgaccgccttctgggaggaaggcttcggcgagctgtttgagaaggccaagcagaacaacaacaaccgcaagaccagcaacggcgacgacagcctgttcttctccaacttctccctgctggggacccctgtgctgaaggacatcaacttcaagatcgagcggggacagctgctggccgtggctggatctacaggcgccggaaaaaccagcctgctcatggtcatcatgggcgagctggaacctagcgagggcaagatcaagcacagcggcaggatcagcttctgtagccagttctcctggatcatgcccggcaccatcaaagagaacatcatcttcggcgtgtcctacgacgagtacagataccgcagcgtgatcaaggcctgccagctggaagaggacatcagcaagttcgccgagaaggacaacatcgtgctcggcgaaggcggcatcacactgtctggcggacagagggccagaatctctctggctagagccgtgtacaaggacgccgatctgtacctgctggatagccccttcggctacctggatgtgctgaccgagaaagagatcttcgagagctgcgtgtgcaagctgatggccaacaagacccggatcctggtcacctccaagatggaacacctgaagaaggccgacaagatcctgattctgcacgagggcagcagctacttttacggcaccttcagcgagctgcagaacctgcagcctgacttcagcagcaaactgatgggctgcgactccttcgaccagttcagcgccgagcggagaaacagcatcctgacagagacactgcaccggttctccctggaaggcgacgctcctgtgtcttggaccgagacaaagaagcagagcttcaagcagaccggcgagtttggcgagaagcggaagaactccattctgaaccccatcaactccatccggaagttcagcatcgtccagaaaacccctctgcagatgaacggcatcgaagaggatagcgacgagcccctggaaagacggctgtctctggtgcctgatagcgaacagggcgaagccatcctgcctcggatctccgtgattagcacaggccctacactgcaggctcggagaaggcagagtgtgctgaacctgatgacccacagcgtgaaccagggacagaatatccacagaaagaccaccgccagcacacggaaagtgtcactggcccctcaggccaacctgactgagctggacatctacagcagacggctgagccaagagacaggcctggaaatcagcgaggaaatcaacgaagaggacctgaaagagtgctttttcgacgacatggaatctatccccgccgtgacaacctggaatacctacctgcggtacatcaccgtgcacaagtccctgatcttcgtgctgatctggtgtctcgtgatcttcctggccgaagtggccgcttctctggtggttctgtggctgctcggaaacaccccactgcaggacaagggcaatagcacccacagccggaacaacagctacgccgtgatcatcacctccaccagctcctactacgtgttctacatctacgtgggcgtcgccgacactctgctcgccatgggattctttagaggactgcccctggtgcacaccctgatcaccgtgtctaagatcctgcaccataagatgctgcacagcgtcctgcaggcccctatgagcacactgaacaccctgaaagccggcggaatcctgaacagattcagcaaggacattgccatcctggacgacctgctgcctctgaccatcttcgacttcatccagctgctgctgatcgtgatcggcgccattgctgtggtggctgtgctgcagccttacatcttcgtggccaccgtgcctgtgatcgtggccttcattatgctgcgggcctactttctgcagacctctcagcagctgaagcagctcgagtctgagggcagaagccccatctttacccacctcgtgaccagcctgaaaggcctgtggaccctgagagcctttggcagacagccctacttcgagacactgttccacaaggccctgaacctgcacaccgccaactggtttctgtatctgagcaccctgcggtggttccagatgaggatcgagatgattttcgtcatcttctttatcgccgtgaccttcatcagcatcctcaccactggcgaaggcgagggcagagtgggaatcattctgaccctggccatgaacatcatgtccacactccagtgggccgtgaacagcagcatcgatgtggacagcctgatgcggagcgtgtcccgggtgttcaagttcatcgacatgcccacagagggcaagcccaccaagagcaccaagccttacaagaacggccagctgagcaaagtcatgatcatcgagaactcccacgtcaagaaggacgacatttggcccagcggaggccagatgaccgtgaaggatctgaccgccaagtacaccgaaggcggaaacgccattctggaaaacatcagctttagcatcagccctggccagcgcgtgggactccttggaagaaccggaagcggcaagtctactctgctgagcgccttcctgagactgctgaataccgagggcgagatccagatcgatggggtgtcctgggacagcatcaccctgcaacaatggcggaaggcctttggcgtgatccctcagaaggtgttcattttcagcggcacgttccggaagaatctggacccctacgagcagtggagcgaccaagagatttggaaggtggccgatgaagtgggactgagaagcgtgatcgagcagtttcccggcaagctggatttcgtgctggtggatggcggctgtgtgctgtctcacggacacaagcagctgatgtgcctggccagatccgtgctgtccaaggccaagattctgctgctcgacgagcctagcgctcacctggatcctgtgacctaccagatcatccggcggacactgaagcaggcctttgccgattgcaccgtgatcctgtgcgagcacagaattgaggccatgctggaatgccagcagtttctggttatcgaagagaacaaagtgcggcagtacgacagcatccagaagctgctgaacgagcggagcctgttcagacaggccatctctcccagcgacagagtgaagctgttccctcaccggaacagctccaagtgcaagagcaagcctcagatcgccgctctgaaagaagaaaccgaggaagaggtgcaggacacacggctgggcggcagctacccttacgatgtgcctgattatgccggcagcggcgctacaaactttagccttctgaagcaggccggggatgtcgaagagaatcctggtccaatgaacagcttcagcaccagcgcctttggacccgtggccttttctctgggactgctgctggttctgcctgccgcttttcctgctcctgtgttcaccctggaagatttcgtcggcgattggagacagaccgccggctacaatctggaccaggtgttggagcaaggcggcgtgtccagcctgtttcagaacctgggagtgtccgtgacacccatccagagaatcgtgctgagcggcgagaacggcctgaagatcgacatccacgtgatcatcccttacgagggcctgtccggcgatcagatgggacagatcgagaagatctttaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacggcaacaagatcatcgacgagcggctgatcaaccccgatggcagcctgctgttcagagtgaccatcaacggcgtgacaggatggcggctgtgcgagagaattctggcctaagcttccactggattgtacaattacataaaataaaatatctttattttcattacatctgtgtgttggttttttgtgtgcgctactaacctgagaccgatctgttgatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacaggtctcaaaccctc

SEQ ID NO: 17 - DNA, 8186 bp, Synthetic construct
  source 1..8186: mol_type = other DNA
ttcgctctgctgcccaggctggaatgcagtggcatgaacactgttcactgcagcctcgacctcctgagctcaagcaatcctcccacctcagcctcctgagtagctgggactacaggtgcatgccactatgcaggctaattttaaaatttgttgagatggattctcgttgcattgcccaggctgatctcaaacttctggtctaaaatgaacctcctgcctcagcctcccgaagtggtgagattataggcataagccactgcaccaggctagtgattgctaacacttaaatagcacttccaagtgccttgacacttttctaaacactttgtattcactaattcgtgtaattctcactatagttttttaaggtaaatactatcgttatctccattttatagatgaggaaactgaggaacaggaagatcatataaactgcccaaggtcatagatgtggaacattctgaactaacaccagactattgggccctatggtggtgctcagatgttacttggaaagtataatgctcacagaagcactctctctctctaggggaatacttgtttccatcatttgttttactctctgtagagggaaacattcctttccaaaggagatttcatcttctgactagtaaatcagactcagaatgagctatgattcatttacaggtaagtcacaagctttattcctggccagataagaaatcagtaatttggttttgtctgaagagatttgggaattcccccaagatgatttcctggagtgggtcagaatgccacctacacactaggttaatgacttcagtgttctgcttggttcgctgatgacaggaaactggtaaaacaaagagggtgagaaaccagctgtatcacagtaataaatattactcagatttaggatcttcttcctagttcctgagtttttttaactttcaagctttcactcagacaatatgcaatgattagctaatacagtttgttttagtattacaaatgatgatggcagattttgcggggaatttgttttaaagttaatgttatcatatttttacaattcttgttttcaatagtttgaaaaagttctgttgggatcctgaattcttaattttgactgagatctagtgagattagttgtctcttttggagataattttagaggcataaaaatggtaaatcaattttttttcaattattagaaatcatgtgcacaaaatagtcaaggggaaagattagcaactgcttttttaatatagcattaattgatttcacaaatagttactatgctgctactgtattttttaggcatcagagatagagttgtgaggaagacaaggcaagatccttgacctccaaatgtttatagtctagcaagataaattaataaatatataattaagtaataggtaatgataaattctataaaggaggccttagatgtgtcggcaataaagtaatcaggccaaaatttttactttcctttgaatttttcaattcaaacacaatgtatgcttgcttttacacagtagggttcagggattagagggttggctctttaaaaaccgtcagagacacaggcaatcctacacaaaattctcagaaggaaggcgcctacgcctgggaatgcccagatgcccctcagagagttgaagatggcgtttctctgagtcaggtcaaagttaacacattaccttcgcttcaaagactgcttggcttcctttcggtggattagtcaagatgttttgctgactgagactaggaaatctataggagggcgggttagtttacattgttccttgtcattatcgctaaaacactccaaagccttccttaaaaatgcgcactgggctaaaaaggatagacaaggaacacatcctgggccggtaattacgcaaagcattatctcctcttacctccttgcagatttttttttctctttcagtacgtgtcctaagatttctgtgccacccttggagttcactcacctaaacctgaaactaataaagcttggttcttttctccgacacgcaaaggaagcgctaaggtaaatgcatcagacccacactgccgcggaacttttcggctctctaaggctgtattttgatatacgaaaggcacattttccttcccttttcaaaatgcaccttgcaaacgtaacaggaacccgactaggatcatcgggaaaaggaggaggaggaggaaggcaggctccggggaagctggtggcagcgggtcctgggtctggcggaccctgacgcgaaggagggtctaggaagctctccggggagccggttctcccgccggtggcttcttctgtcctccagcgttgccaactggacctaaagagaggccgcgactgtcgcccacctgcgggatgggcctggtgctgggcggtaaggacacggacctggaaggagcgcgcgcgagggagggaggctgggagtcagaatcgggaaagggaggtgcggggcggcgagggagcgaaggaggagaggaggaaggagcgggaggggtgctggcgggggtgcgtagtgggtggagaaagccgctagagcaaatttggggccggaccaggcagcactcggcttttaacctgggcagtgaaggcgggggaaagagcaaaaggaaggggtggtgtgcggagtaggggtgggtggggggaattggaagcaaatgacatcacagcaggtcagagaaaaagggttgagcggcaggcacccagagtagtaggtctttggcattaggagcttgagcccagacggccctagcagggaccccagcgcccgagatacccagaggtcgcctctggaaaaggccagcgttgtctccaaactttttttcaggtgagaaggtggccaaccgagcttcggaaagacacgtgcccacgaaagaggagggcgtgtgtatgggttgggtttggggcagataagtttgtacaaaaaagagaggtgccaccatgcagagaagccctctggaaaaggccagcgtggtgtccaagctgttcttcagctggacccggcctatcctgcggaagggctatagacagagactggaactgagcgacatctatcagatccccagcgtggacagcgccgacaacctgtctgagaagctggaaagagagtgggacagagagctggcctccaagaagaaccccaagctgatcaacgccctgcggcggtgcttcttctggcggtttatgttctacggcatcttcctgtacctgggcgaagtgaccaaagccgtgcagcctctgctgctgggcagaatcattgccagctacgaccccgacaacaaagaggaacggtctatcgccatctacctcggcatcggcctgtgcctgctgtttatcgtcagaaccctgctgctgcaccccgccatctttggactgcaccacatcggcatgcagatgcggatcgccatgttcagcctgatctacaagaaaaccctgaagctgtccagcagagtgctggacaagatcagcatcggacagctggtgtccctgctgagcaacaacctgaacaagttcgacgaaggcctggctctggcccacttcgtgtggattgctcctctgcaagtggccctgctgatgggcctgatttgggaactgctgcaggccagcgccttttgcggactgggatttctgattgtgctggccctgttccaggccggactggggagaatgatgatgaagtaccgggaccagagagccggcaagatctccgagagactggtcatcaccagcgagatgatcgagaacatccagagcgtgaaggcctactgctgggaagaggccatggaaaagatgattgagaatctgcggcagaccgagctgaagctgacaagaaaggccgcctacgtgcgctacttcaacagcagcgccttcttcttctccggcttcttcgtggtgttcctgagcgtgctgccctacgctctgatcaagggcatcatcctgagaaagattttcaccaccatttctttctgcatcgtgctgcggatggccgtgaccagacagtttccttgggctgtgcagacttggtacgatagcctgggcgccatcaacaagatccaggacttcctgcagaagcaagagtacaagaccctcgagtacaacctgaccaccaccgaggtggtcatggaaaacgtgaccgccttctgggaggaaggcttcggcgagctgtttgagaaggccaagcagaacaacaacaaccgcaagaccagcaacggcgacgacagcctgttcttctccaacttctccctgctggggacccctgtgctgaaggacatcaacttcaagatcgagcggggacagctgctggccgtggctggatctacaggcgccggaaaaaccagcctgctcatggtcatcatgggcgagctggaacctagcgagggcaagatcaagcacagcggcaggatcagcttctgtagccagttctcctggatcatgcccggcaccatcaaagagaacatcatcttcggcgtgtcctacgacgagtacagataccgcagcgtgatcaaggcctgccagctggaagaggacatcagcaagttcgccgagaaggacaacatcgtgctcggcgaaggcggcatcacactgtctggcggacagagggccagaatctctctggctagagccgtgtacaaggacgccgatctgtacctgctggatagccccttcggctacctggatgtgctgaccgagaaagagatcttcgagagctgcgtgtgcaagctgatggccaacaagacccggatcctggtcacctccaagatggaacacctgaagaaggccgacaagatcctgattctgcacgagggcagcagctacttttacggcaccttcagcgagctgcagaacctgcagcctgacttcagcagcaaactgatgggctgcgactccttcgaccagttcagcgccgagcggagaaacagcatcctgacagagacactgcaccggttctccctggaaggcgacgctcctgtgtcttggaccgagacaaagaagcagagcttcaagcagaccggcgagtttggcgagaagcggaagaactccattctgaaccccatcaactccatccggaagttcagcatcgtccagaaaacccctctgcagatgaacggcatcgaagaggatagcgacgagcccctggaaagacggctgtctctggtgcctgatagcgaacagggcgaagccatcctgcctcggatctccgtgattagcacaggccctacactgcaggctcggagaaggcagagtgtgctgaacctgatgacccacagcgtgaaccagggacagaatatccacagaaagaccaccgccagcacacggaaagtgtcactggcccctcaggccaacctgactgagctggacatctacagcagacggctgagccaagagacaggcctggaaatcagcgaggaaatcaacgaagaggacctgaaagagtgctttttcgacgacatggaatctatccccgccgtgacaacctggaatacctacctgcggtacatcaccgtgcacaagtccctgatcttcgtgctgatctggtgtctcgtgatcttcctggccgaagtggccgcttctctggtggttctgtggctgctcggaaacaccccactgcaggacaagggcaatagcacccacagccggaacaacagctacgccgtgatcatcacctccaccagctcctactacgtgttctacatctacgtgggcgtcgccgacactctgctcgccatgggattctttagaggactgcccctggtgcacaccctgatcaccgtgtctaagatcctgcaccataagatgctgcacagcgtcctgcaggcccctatgagcacactgaacaccctgaaagccggcggaatcctgaacagattcagcaaggacattgccatcctggacgacctgctgcctctgaccatcttcgacttcatccagctgctgctgatcgtgatcggcgccattgctgtggtggctgtgctgcagccttacatcttcgtggccaccgtgcctgtgatcgtggccttcattatgctgcgggcctactttctgcagacctctcagcagctgaagcagctcgagtctgagggcagaagccccatctttacccacctcgtgaccagcctgaaaggcctgtggaccctgagagcctttggcagacagccctacttcgagacactgttccacaaggccctgaacctgcacaccgccaactggtttctgtatctgagcaccctgcggtggttccagatgaggatcgagatgattttcgtcatcttctttatcgccgtgaccttcatcagcatcctcaccactggcgaaggcgagggcagagtgggaatcattctgaccctggccatgaacatcatgtccacactccagtgggccgtgaacagcagcatcgatgtggacagcctgatgcggagcgtgtcccgggtgttcaagttcatcgacatgcccacagagggcaagcccaccaagagcaccaagccttacaagaacggccagctgagcaaagtcatgatcatcgagaactcccacgtcaagaaggacgacatttggcccagcggaggccagatgaccgtgaaggatctgaccgccaagtacaccgaaggcggaaacgccattctggaaaacatcagctttagcatcagccctggccagcgcgtgggactccttggaagaaccggaagcggcaagtctactctgctgagcgccttcctgagactgctgaataccgagggcgagatccagatcgatggggtgtcctgggacagcatcaccctgcaacaatggcggaaggcctttggcgtgatccctcagaaggtgttcattttcagcggcacgttccggaagaatctggacccctacgagcagtggagcgaccaagagatttggaaggtggccgatgaagtgggactgagaagcgtgatcgagcagtttcccggcaagctggatttcgtgctggtggatggcggctgtgtgctgtctcacggacacaagcagctgatgtgcctggccagatccgtgctgtccaaggccaagattctgctgctcgacgagcctagcgctcacctggatcctgtgacctaccagatcatccggcggacactgaagcaggcctttgccgattgcaccgtgatcctgtgcgagcacagaattgaggccatgctggaatgccagcagtttctggttatcgaagagaacaaagtgcggcagtacgacagcatccagaagctgctgaacgagcggagcctgttcagacaggccatctctcccagcgacagagtgaagctgttccctcaccggaacagctccaagtgcaagagcaagcctcagatcgccgctctgaaagaagaaaccgaggaagaggtgcaggacacacggctgggcggcagctacccttacgatgtgcctgattatgccggcagcggcgctacaaactttagccttctgaagcaggccggggatgtcgaagagaatcctggtccaatgaacagcttcagcaccagcgcctttggacccgtggccttttctctgggactgctgctggttctgcctgccgcttttcctgctcctgtgttcaccctggaagatttcgtcggcgattggagacagaccgccggctacaatctggaccaggtgttggagcaaggcggcgtgtccagcctgtttcagaacctgggagtgtccgtgacacccatccagagaatcgtgctgagcggcgagaacggcctgaagatcgacatccacgtgatcatcccttacgagggcctgtccggcgatcagatgggacagatcgagaagatctttaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacggcaacaagatcatcgacgagcggctgatcaaccccgatggcagcctgctgttcagagtgaccatcaacggcgtgacaggatggcggctgtgcgagagaattctggcctaagcttccactggattgtacaattacataaaataaaatatctttattttcattacatctgtgtgttggttttttgtgtgcgctactaaccctc